(19) United States
(12) Patent Application Publication
(10) Pub. No.: US 2011/0067124 A1
(43) Pub. Date: Mar. 17, 2011

(54) DETECTION AND TREATMENT OF DRUG ASSOCIATED ANGIOEDEMA

(76) Inventor: GEORG DEWALD, Bonn (DE)

(21) Appl. No.: 12/876,946

(22) Filed: Sep. 7, 2010

Related U.S. Application Data
(63) Continuation of application No. 12/126,574, filed on May 23, 2008, now abandoned.
Application No. 12/126,574 is a continuation-in-part of application No. PCT/EP2006/011245, filed on Nov. 23, 2006.

(30) Foreign Application Priority Data
Nov. 23, 2005 (DE) 05 02 5588.4

(51) Int. Cl. A01K 67/027; C12Q 1/68; G01N 33/573; C12N 9/50; C12N 15/57

(52) U.S. Cl. 800/13; 435/6; 435/23; 435/7.4; 435/219; 536/23.53

(57) ABSTRACT

The present invention relates to an in vitro method of diagnosing a drug-associated angioedema or a predisposition thereto in a subject being suspected to having developed or of having a predisposition to develop a drug-associated angioedema or in a subject being intended to be treated with a drug associated with the development of angioedema, the method comprising determining in a biological sample from said subject the presence or absence of a disease-associated mutation in a nucleic acid molecule regulating the expression of or encoding coagulation factor XII; wherein the presence of such a mutation is indicative of a drug-associated angioedema or a predisposition thereto.

The present invention relates to an in vitro method of diagnosing a drug-associated angioedema or a predisposition thereto in a subject being suspected of having developed or of having a predisposition to develop a drug-associated angioedema or in a subject being suspected of being a carrier for a drug-associated angioedema or in a subject being intended to be treated with a drug associated with the development of angioedema, the method comprising determining in a biological sample from said subject the presence or absence of a disease-associated mutation in a nucleic acid molecule regulating the expression of or encoding coagulation factor XII; wherein the presence of such a mutation is indicative of a drug-associated angioedema or a predisposition thereto. The present invention also relates to a method of diagnosing a drug-associated angioedema or a predisposition thereto in a subject being suspected of having developed or of having a predisposition to develop a drug-associated angioedema or in a subject being suspected of being a carrier for a drug-associated angioedema or in a subject being intended to be treated with a drug associated with the development of angioedema, the method comprising assessing the presence, amount and/or activity of coagulation factor XII in said subject and including the steps of: (a) determining from a biological sample of said subject in vitro, the presence, amount and/or activity of: (i) a (poly)peptide encoded by the coagulation factor XII gene; (ii) a substrate of the (poly)peptide of (i); or (iii) a (poly)peptide processed by the substrate mentioned in (ii); (b) comparing said presence, amount and/or activity with that determined from a reference sample; and (c) diagnosing, based on the difference between the samples compared in step (b), the pathological condition of a drug-associated angioedema or a predisposition thereto. The present invention also relates to a method of identifying a compound modulating coagulation factor XII activity which is suitable as a medicament or a lead compound for a medicament for the treatment and/or prevention of drug-associated angioedema, the method comprising the steps of: (a) in vitro contacting a coagulation factor XII (poly)peptide or a functionally related (poly)peptide with the potential modulator; and (b) testing for modulation of coagulation factor XII activity, wherein modulation of coagulation factor XII activity is indicative of a compound's suitability as a medicament or a lead compound for a medicament for the treatment and/or prevention of drug-associated angioedema. Furthermore, the present invention relates to methods of gene therapy and to a kit for diagnosing drug-associated angioedema.

Several documents are cited throughout the text of this specification. The disclosure content of the documents cited herein (including any manufacture's specifications, instructions, etc.) is herewith incorporated by reference.

All or any combination of steps (including single steps only) carried out in the method of the present invention and cited throughout this specification can be carried out in any combination of in vivo, ex vivo or in vitro.

Angioedema is a symptom of numerous disease entities (Greaves & Lawlor 1991, J. Am. Acad. Dermatol. 25: 155-165). In large it can be inherited or aquired. Inherited types are the well known hereditary angioedema types I and II (Nzeako et al. 2001, Arch. Intern. Med. 161: 2417-2429), both related to a complement C1 inhibitor deficiency, and the recently described type III (Bork et al. 2000, Lancet 356:213-217). One form of aquired angioedema is caused by autoantibody formation against C1-inhibitor. Another very important group of aquired angioedema is associated with drug administration (Nettis et al. 2001, Immunopharmacol. Immunotoxicol. 23: 585-595), angioedema thus representing an adverse drug reaction. This latter group is of special interest because of its high frequency and medical implications regarding the treatment of various common disease conditions.

Among the numerous drugs which are associated with the development of angioedema symptoms there are certain classes of drugs, where this adverse reaction is considered to be due to some kind of interaction of the drug with various components of the kinin system and/or related systems. These interactions may be direct or indirect interactions; finally, in many cases they appear to result in increased levels of a vasoactive kinin. For example, because angiotensin-converting enzyme (ACE) is involved in the degradation of the vasoactive peptide bradykinin, an inhibitor of angiotensin-converting enzyme (ACE), as used for the treatment of hypertension and other conditions, can lead to an accumulation of bradykinin, which in turn may cause an increased risk for angioedema development (vide infra).

The following classes of drugs [classes (a) to (I)], which partly represent drugs already in routine clinical use and partly drugs presently in clinical or preclinical development, are of special interest for the present invention's teaching:

(a) angiotensin-converting enzyme (ACE) inhibitors; (b) angiotensin II receptor type 1 (AT₁) antagonists (sartans); (c) fibrinolytic or thrombolytic drugs (recombinant tissue plasminogen activator (rtPA; alteplase), urokinase, streptokinase, and related drugs or medications); (d) vasopeptidase inhibitors; (e) neutral endopeptidase (NEP) inhibitors; (f) inhibitors of endothelin-converting enzyme 1 (ECE-1); (g) triple inhibitors of ECE-1, NEP and ACE; (h) inhibitors of dipeptidyl peptidase IV (DPP IV); (i) calcium channel blockers; (j) estrogens and estrogen-like drugs; (k) anti-androgens; and (I) corticosteroids. Among these drug classes some are known to be associated with the development of angioedema, for others it is envisaged that they may also be associated with an increased risk of developing angioedema.

As pointed out above, certain drugs or medicaments when administered to a patient have been observed to be associated with a risk of developing angioedema. It is appreciated that angioedema is an adverse drug reaction that usually affects only a small percentage of all the individuals to whom one of the aforementioned drugs is administered; for most drugs considered here this percentage might range from less than 1% to 5%.

Although the occurrence of angioedema is thus usually a rare event, this event can nevertheless have very important consequences because of the potentially life-threatening nature of an angioedema attack when manifesting as a laryngeal edema. Therefore, it would be highly desirable, to be in a position to predict, if a patient is at risk for developing this particular adverse drug reaction. The availability of an appropriate diagnostic test, for example a pharmacogenetic marker, would be of great value in allowing to identify individuals who carry an increased risk, a predisposition for the development of drug-associated angioedema. If it is intended, for example, to treat a patient suffering from hypertension with an ACE inhibitor, it will be important to know if a disease-associated mutation is present in this patient. If this is the case, for example a differential type of medication could be chosen.

Such a test may also be valuable with respect to a patient already being affected by a drug-associated angioedema. In such a case a positive test result, for example the recognition of the presence of a positively predicting disease-associated marker could make it possible to choose an effective specific treatment, namely a treatment targeting the underlying cause.

Further, it would be desirable to be able to specifically prevent the development of a drug-associated angioedema. Therapeutic means and measures, disclosed in the present invention, will also be applicable for the purpose of prevention, for example in a situation in which a patient is positive for a disease-associated mutation but nevertheless, for certain medical reasons, is preferred to be treated or must be treated with a specific drug known to be associated with an increased risk for the development of angioedema.

At present, there is no reliable method of predicting whether a patient will eventually develop angioedema upon treatment with a drug of said classes. In addition, prior to the invention, it was also not possible to determine whether the symptoms of angioedema observed in a patient under treatment with a drug of said classes have developed or might have developed due to administration of such a drug, and/or to determine a precise cause for the development of angioedema in such a patient. Further, it is presently not possible to effectively treat patients with a drug-associated angioedema as the specific underlying cause for the onset of this disease or reaction in many cases is not known. Therefore, there was an urgent need to develop the teaching of the present invention.

Thus, the technical problem underlying the present invention was to provide means and methods for predicting the risk of and for diagnosis, prevention and treatment of drug-associated angioedema.

The solution to this technical problem is achieved by providing the embodiments characterized in the claims.

Accordingly, the present invention relates to an in vitro method of diagnosing a drug-associated angioedema or a predisposition thereto in a subject being suspected of having developed or of having a predisposition to develop a drug-associated angioedema or in a subject being suspected of being a carrier for a drug-associated angioedema or in a subject being intended to be treated with a drug associated with the development of angioedema, the method comprising determining in a biological sample from said subject the presence or absence of a disease-associated mutation in a nucleic acid molecule regulating the expression of or encoding coagulation factor XII; wherein the presence of such a mutation is indicative of a drug-associated angioedema or a predisposition thereto.

The term “nucleic acid” or “nucleic acid molecule” refers to DNA or RNA, including genomic DNA, cDNA, mRNA, hnRNA etc as well as chimeras thereof. Included are artificially modified nucleic acid molecules carrying chemically modified bases. All nucleic acid molecules may be either single or double stranded.

In principle, the detection of at least one disease-associated mutation, such as 2, 3, 4, 5, 6, 7, 8, 9, 10 or more mutations or combinations of various different mutations, in at least one allele is an indication that the subject to be diagnosed either with respect to a potentially existing disease predisposition or susceptibility or because of being affected by the disease is a carrier. In general, if a disease-associated mutation is dominant, it may be causative for determining a disease predisposition and/or for the onset or progress of the disease and a diagnosis of heterozygosity as only of its presence in the genome at all, will be indicative of the subject being prone to developing the disease if it does not already suffer from it. A recessive character of a mutation will more likely indicate that only its homozygous occurrence will have a direct impact on the onset or progress of the disease, whereas its occurrence in heterozygous form will rather qualify the subject as a carrier only, unless other concomitantly occurring mutations contribute to the onset or progress of the disease.

The term “diagnosing” means assessing whether or not an individual or a subject has a specific mutation linked with drug-associated angioedema and concluding from the presence of said mutation that the individual or subject has a predisposition to develop a drug-associated angioedema or is a carrier for drug-associated angioedema and/or has a drug-associated angioedema, preferably a drug-associated angioedema related to a mutation in a nucleic acid molecule regulating the expression of or encoding coagulation factor XII.

The term “angioedema” refers to an abnormality to cause skin swellings, gastrointestinal symptoms (abdominal pain attacks) due to edema of the intestinal wall, or edema of the tongue, or laryngeal edema, which may ultimately result in death by asphyxiation.

The term “drug-associated angioedema” refers to an angioedema or any angioedema-related symptoms including for example also migraine or headache symptoms that manifest in association with the administration of a drug. For the purpose of the present invention “drug” preferably relates to those drugs or classes of drugs that have a direct or indirect influence on the production or degradation or on the bioavailability of a vasoactive kinin or on signaling processes relevant for such kinins. It is important to note that the temporal relationship between the appearance of angioedema and drug administration can show considerable variation. The angioedema can manifest within hours after a first exposure, however, it is also possible that angioedema manifests for the first time with great delay. It is appreciated that drug-associated angioedema usually manifests only in a small percentage of all those individuals being treated with or being exposed to a certain drug. Thus, individual factors have to play an important role for disease manifestation. Among these, specific genetic factors are assumed to be of great importance. In this context it is also of importance that it is well established that there are race-specific differences, likely of genetic nature, regarding the relative frequency of drug-associated angioedema (Brown et al. 1996, Clin. Pharmacol. Ther. 60: 8-13; Gibbs et al. 1999, Br. J. Clin. Pharmacol. 48: 861-865).

It is generally assumed that bradykinin and related kinins can be important mediators of angioedema development (Nussberger et al. 1998, Lancet 351: 1693-1697; Kaplan et al. 2002, J. Allergy Clin. Immunol. 109: 195-209; Cugno et al. 2003, Int. Immunopharmacol. 3: 311-317). Regarding drug-associated angioedema, for example in patients being treated with an ACE inhibitor an increased bradykinin plasma concentration has been observed during angioedema attacks (Nussberger et al. 1998, Lancet 351: 1693-1697; Cugno et al. 2003, Int. Immunopharmacol. 3: 311-317). It has also been considered that substance P, a peptide that is—like neurokinin A—derived from preprotachykinin 1, could be of importance with respect to angioedema associated with the use of ACE inhibitors (Vleeming et al. 1998, Drug Saf. 18: 171-188).

A kinin derived from complement component C2—following an eventually increased or uncontrolled activation of the classical complement pathway—has also been considered to be of pathophysiological significance for angioedema development (Donaldson et al. 1977, Trans. Assoc. Am. Physicians 90: 174-183; Strang et al. 1988, J. Exp. Med. 168: 1685-1698).

Coagulation factor XII plays an important role for the control of processes potentially related to the development of angioedema in that it can influence, for example, the generation of vasoactive kinins from the contact system. It is envisaged, in accordance with the invention, for example, that a mutated coagulation factor XII molecule can be responsible for an abnormally high kinin generation. Further, without being bound by any theory, it is believed, in accordance with the invention, that coagulation factor XII may also occasionally, under certain circumstances, promote the generation of vasoactive kinins from precursor proteins outside the immediate contact system.

The term “predisposition”, in accordance with the present invention, refers to a genetic condition that (a) increases the risk for the development of a disease or promotes or facilitates the development of a disease and/or that (b) facilitates to pass on to the offspring specific alleles of a gene increasing the risk for or promoting the development of such condition or disease.

The term “biological sample”, in accordance with the present invention, relates to the specimen taken from a mammal. Preferably, said specimen is taken from hair, skin, mucosal surfaces, body fluids, including blood, plasma, serum, urine, saliva, sputum, tears, liquor cerebrospinalis, semen, synovial fluid, amniotic fluid, breast milk, lymph, pulmonary sputum, bronchial secretion, or stool.

The term “mutation” comprises, inter alia, substitutions, additions, insertions, inversions, duplications or deletions within nucleic acid molecules, wherein one or more nucleotide positions can be affected by a mutation. These mutations occur with respect to the wild-type nucleic acid sequence. As the “wild-type” or “normal” nucleic acid sequence of the coagulation factor XII gene is considered herein the sequence (bases 1 to 10616) given under GenBank acc. no. AF 538691 and, with respect to extended flanking sequences, the sequence given in the July 2003 human reference sequence of the UCSC Genome Browser, v. 53 (vide infra). A mutation may affect preferably up to 1, 2, 3, 4, 5, 6, 7, 8, 9, 10 or even of up to 20, 30, 40, 50, or up to 1000 nucleotides. However, it is also conceivable that even larger sequences are affected. Therefore, the term “mutation” also relates to, e.g., a nucleotide deletion, substitution or insertion of up to 10000 or up to 20000 nucleotides, also comprising the situation when the entire coding, non-coding and/or regulating sequence of a gene is affected. Mutations can involve coding or non-coding gene regions. The term “non-coding” preferably relates to introns, to the non-coding parts of exons, to 5′- and 3′-flanking regulatory sequences, thus also to expression control sequences including control elements such as promoter, enhancer, silencer, transcription terminator, polyadenylation site. It is well known to the person skilled in the art that mutations in these regions of a gene can have a substantial impact on gene expression, eventually also with respect to specific tissues. For example, mutations in these sites can result in a nearly complete shut-down of gene expression or in a drastic overexpression. However, mutations in non-coding regions can also exert important effects by altering the splicing process; such mutations, for example, can affect the intron consensus sequences at the splice and branch sites, sometimes they activate cryptic sites, or create ectopic splice sites.

On the other hand, a mutation can also reside in the coding region of a gene and severely affect the protein's structural and/or functional characteristics, for example by causing amino acid substitutions. However, even so-called silent or synonymous mutations must not necessarily be silent. For example, mutations within exonic splicing enhancers or silencers may affect mRNA splicing, which may for example alter protein structure or cause phenotypic variability and variable penetrance of mutations elsewhere in the gene (Liu H.-X. et al. 2001, Nature Genet. 27: 55-58; Blencowe 2000, TIBS 25: 106-110; Verlaan et al. 2002, Am. J. Hum. Genet. 70; Pagani et al. 2003, Hum. Mol. Genet. 12: 1111-1120).

However, it is well known in the art that not any deviation from a given reference sequence must necessarily result in a disease condition or a predisposition thereto. For example the gene encoding human coagulation factor XII is known to occur in a number of variations comprising polymorphisms such as those deposited in the databank of Seattle (http://pga.gs.washington.edu, University of Washington, ‘Seattle SNPs’).

The term “polymorphism” or “polymorphic variant” means a common variation in the sequence of DNA among individuals (NHGRI glossary). “Common” means that there are two or more alleles that are each present at a frequency of at least 1% in a population. Usually it is understood, that polymorphisms, or at least the majority of polymorphisms, represent variations that are benign, functionally neutral, not having an adverse effect on gene function. However, it is also clear that polymorphic variants exist which can have an impact with respect to the development of a disease. This impact can be not only a disease-predisposing one, but, in certain cases, it can also be a protective effect reducing the risk of disease manifestation.

Taking into account the existence of polymorphic variants, it is reasonable to consider the existence of numerous alternative wild-type sequences. For various purposes of the present invention, for example for the design of nucleotide probes and primers and also for the design of oligonucleotides to be used therapeutically, it will be important to carefully take into account the existence of such variant sequences.

Although the term “mutation” basically describes any alteration or change in a gene from its natural state, it is often understood as a disease-causing change, as a change that causes a disorder or the inherited susceptibility to a disorder. For the skilled artisan and under certain circumstances, the terms “polymorphic variant” (“polymorphism”) and “mutation” have the same connotation and refer to the same molecular phenomenon, namely alteration in or deviation from a paradigmatic wild-type sequence.

For the purpose of the present invention, the term “disease-associated mutation” refers to a mutation in a nucleic acid molecule regulating the expression of or encoding coagulation factor XII and which is linked with drug-associated angioedema and/or a predisposition for drug-associated angioedema. In accordance with the present invention, a “disease-associated mutation” is preferably a rare mutation, preferably with a frequency<1%, and more preferably a mutation with an important disease-causing effect, eventually even a dominant mutation. Nevertheless, in accordance with the present invention, it is also envisaged that polymorphic variants exist that can have an influence on disease predisposition and/or the onset or progress of the disease (vide infra), and which, thus, also represent a “disease-associated mutation”.

It is important to note that an affected individual may carry more than one disease-associated mutation. In order to determine whether or not a mutation is disease-associated, the person skilled in the art may, for example, compare the frequency of a specific sequence change in patients affected by the disease, in this case having developed an angioedema in association with the administration of a particular drug, with the frequency of this sequence change in appropriately chosen control individuals, and conclude from a statistically significantly deviating frequency in the patient group that said mutation is a disease-associated mutation. The person skilled in the art knows how to design such a comparison of patients and controls. For example, patients and controls should be carefully matched, for example for age, sex, and ethnicity. Controls could be individuals assumed to be healthy, like blood donors, but also a population-based control sample appears to be possible, although it is appreciated that among such samples there might be a small percentage of individuals included who have a predisposition for the disease. Thus, if one would study e.g. a group of patients affected by angioedema attacks in association with the administration of an ACE inhibitor, it would be desirable to use as unaffected or healthy controls individuals who have been treated with an ACE inhibitor for prolonged periods (at least for periods comparable to the treatment periods in patients) without having developed any angioedema symptoms.

According to the present invention, the term “statistically significant” describes a mathematical measure of difference between groups. The difference is said to be statistically significant if it is greater than what might be expected to happen by chance alone. Preferably, a P-value<0.10, more preferred a P-value<0.05, even more preferred, a P-value<0.01, calculated without using any corrections, like those for multiple testing, is considered to be indicative of a significant difference.

In cases where more than one mutation is present in a nucleic acid molecule, wherein said mutation is linked with drug-associated angioedema, it may suffice to detect the presence of one mutation only or of a lower number of mutations than are actually present in the nucleic acid molecule and associated with drug-associated angioedema. Normally, it is not relevant for the purpose of diagnosis, whether such associated mutations are solely indicative (thus having for example a bystander effect) and not causative or whether they are causative for the disease predisposition or the onset or progress of the disease.

GenBank accession number AF538691 lists a consensus sequence of the human coagulation factor XII gene and a number of polymorphic variants observed in Caucasian and Negroid individuals. For a large part, these and potentially existing other polymorphic variants may be functionally neutral. Nevertheless, it is possible that at least some polymorphic variants are not neutral, i.e. that they can exhibit functional, quantitative or qualitative consequences like for example influencing directly the susceptibility or predisposition for the development of drug-associated angioedema or modulating the pathogenic effect of another mutation associated with drug-associated angioedema.

For example, it is envisaged that a common polymorphism (46C/T) in the 5′-UTR (in exon 1) of the human coagulation factor XII gene can be of importance for the present invention. It is known that this polymorphism is significantly associated with the plasma concentration of coagulation factor XII (Kanaji et al. 1998, Blood 91: 2010-2014), the T allele being associated with a decreased translation efficiency; in functional and antigenic assays, individuals with the genotype C/C show 170% of the concentration seen in pooled normal plasma, whereas in individuals with the genotype T/T the factor XII plasma concentration is 80% of that seen in pooled normal plasma. In accordance with the present invention, one, therefore, may consider the C allele being a risk factor whose presence can increase the risk for the development of angioedema.

Thus, in a less preferred alternative, it is conceivable that, in fact, some of said polymorphic variants represent a disease-associated mutation (vide supra). It is also envisaged that such a situation might arise from linkage disequilibrium phenomena. With these limitations in mind, the deposited consensus sequence mentioned above, is considered herein to represent the “wild-type” sequence.

It is important to note that the term “nucleic acid molecule regulating the expression of or encoding coagulation factor XII” preferably comprises the complete genomic sequence of the coagulation factor XII gene including extended flanking regulatory sequences (vide infra) as well as sequences or nucleic acid molecules which are physically unrelated to the coagulation factor XII gene but which exert regulatory effects on the expression of coagulation factor XII. The term “nucleic acid molecule regulating the expression of or encoding coagulation factor XII” also refers to portions of the above sequences, for example, the promoter of said gene.

The term “regulating the expression” means influencing, including increasing or decreasing transcription or translation. Accordingly, increasing or decreasing means producing more or less, respectively, RNA or (poly)peptides. The term “regulating the expression” also refers to influencing splicing processes, as well as the tissue-specific expression of a gene. The skilled person knows that expression may be regulated, for example, by enhancer or silencer sequences, splicing signals as well as other sequences which affect splicing processes, binding of transcription factors, polyadenylation sequences, transport signals, transcription terminator and the like. It is also envisaged that nucleic acid sequences physically unrelated to the coagulation factor XII gene locus can participate in the regulation of the expression of coagulation factor XII, and thus may have an impact on the development of angioedema symptoms. For example, a gene locus on the short arm of chromosome 10, around marker D10S1653, envisaged to be located within the nucleotide sequence comprising nucleotides chr10:10,554,416 to chr10:18,725,506 (UCSC Genome Browser/July 2003) has been demonstrated to affect coagulation factor XII plasma level (Soria et al. 2002, Am. J. Hum. Genet. 70:567-574) and may, thus, also affect the predisposition for or the development of a drug-associated angioedema.

Sequences “encoding coagulation factor XII” refer to the coding sequence of the coagulation factor XII gene. Said term relates to the genomic coding sequence as well as the coding sequence in a RNA or cDNA molecule.

The term “coagulation factor XII” relates preferably to coagulation factor XII, which is a serine protease circulating in plasma as a single-chain inactive zymogen of approximately 80 kDa. Particularly preferred in accordance with the present invention is the coagulation factor XII corresponding to the mRNA sequence given under GenBank accession no. NM 000505.2 and encoded by the nucleic acid molecule deposited under GenBank accession number AF538691 which is considered by the present invention as the wild-type coagulation factor XII gene sequence and which includes 5′ promoter sequences (up to 1581 bp upstream from exon 1), coding and non-coding exon sequences, intronic sequences, and 3′ flanking regulatory sequences, including 1598 bp downstream from the end of exon 14 which corresponds to the end of the coagulation factor XII mRNA as given under GenBank accession number NM 000505.2. With respect to genomic sequences further extending into upstream and downstream direction the sequence considered here to represent the wild-type sequence may be taken from the July 2003 human reference sequence of the UCSC Genome Browser, v. 53, namely from the reverse complement sequence of chr5:176,807,093-176,821,530 (representing 4000 bp upstream of exon 1 and 3000 bp downstream of exon 14). The GenBank entry AF538691 relates to the gene of Homo sapiens coagulation factor XII (Hageman factor) (F12) of which several variants are known in the art (vide supra). The term “coagulation factor XII” also relates to sequences with an identity of at least 70%, 75%, 80%, 85%, 90%, 95%, 96%, 97%, 98%, or 99% when compared with the sequence of GenBank accession number AF538691. In addition, the present invention also relates to various protein isoforms corresponding to different transcripts produced by alternative splicing (for example, those shown in: “http://www.ncbi.nih.gov/IEB/Research/Acembly/ay.cgi?db=human&I═F12”). Further, the present invention also relates to species homologues in other animals, preferably mammals including rat, mouse, guinea pig, pig, cattle or rabbit. Polymorphic variants of coagulation factor XII may also comprise variants with large deletions in, for example, intron regions. Said variants may nevertheless encode a coagulation factor XII (poly)peptide of wild-type sequence. It is important to note that when aligned to the sequence of AF538691, the calculated sequence identity may be considerably lower than expected for normal polymorphic variation. Thus, preferred in accordance with the present invention are biologically active variants and also fragments of coagulation factor XII encoded by a nucleic acid molecule with a sequence identity of at least 70%, 75%, 80%, 85%, 90%, 95%, 96%, 97%, 98%, or 99% when compared with the sequence of databank accession number AF538691. Sequence identity may be determined by using the Bestfit® program (Wisconsin Sequence Analysis Package, Version 8 for Unix, Genetics Computer Group, University Research Park, 575 Science Drive, Madison, Wis. 53711). Bestfit® uses the local homology algorithm of Smith and Waterman to find the best segment of homology between two sequences (Advances in Applied Mathematics 2:482-489 (1981)). When using Bestfit® or any other sequence alignment program to determine whether a particular sequence is, for instance, 95% identical to a reference sequence, the parameters are set, of course, such that the percentage of identity is calculated over the full length of the reference nucleotide sequence and that gaps in homology of up to 5% of the total number of nucleotides in the reference sequence are allowed. The identity between a first sequence and a second sequence, also referred to as a global sequence alignment, is determined using the FASTDB computer program based on the algorithm of Brutlag and colleagues (Comp. App. Biosci. 6:237-245 (1990)). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

The present invention is related to the observation that administration of certain drugs can induce or might induce—at least in a number of patients—the development of angioedema attacks, thus a drug-associated angioedema. Examples relevant for the present invention are drugs of the following classes: (a) angiotensin-converting enzyme (ACE) inhibitors; (b) angiotensin II receptor type 1 (AT₁) antagonists (sartans); (c) fibrinolytic or thrombolytic drugs; (d) vasopeptidase inhibitors; (e) neutral endopeptidase (NEP) inhibitors; (f) inhibitors of endothelin-converting enzyme 1 (ECE-1); (g) triple inhibitors of ECE-1, NEP and ACE; (h) inhibitors of dipeptidyl peptidase IV (DPP IV); (i) calcium channel blockers; (j) estrogens and estrogen-like drugs; (k) anti-androgens; and (I) corticosteroids.

It is believed in accordance with the present invention, that additional drugs or drug classes, partly under development or to be developed, may also be associated with an increased risk for angioedema development due to kinin-related effects and can thus be of importance, too, with respect to the present invention's disclosure. Such drugs could be for example bradykinin receptor agonists, inhibitors of endopeptidase 24.16 (=neurolysin), inhibitors of endopeptidase 24.15 (thimet oligopeptidase), or meprin A inhibitors.

According to the present invention, the symptoms observed in patients affected by a drug-associated angioedema can be associated with mutations in a nucleic acid molecule regulating the expression of or encoding coagulation factor XII.

Such mutations may comprise for example, but are not limited to (1) a mutation that favours, directly or indirectly, the production of a vasoactive kinin, (2) a mutation that alters the interaction of coagulation factor XII with activating surfaces or with a cell surface receptor or a cell surface receptor complex or with another physiologically interacting molecule, (3) a mutation that results in an increased stability of coagulation factor XII and/or an increased stability of its mRNA, (4) a mutation that results in an increased activity of coagulation factor XII, (5) a mutation that results in an alteration of substrate specificity of coagulation factor XII, (6) a mutation that results in an aberrant proteolytic processing of coagulation factor XII or (7) a mutation that results in an irregular interaction with C1 esterase inhibitor.

Further, without being bound by any theory, it is believed in accordance with the invention, that certain mutations or variations within certain regions of the coagulation factor XII gene may be mutations that affect the splicing, the expression, the structure and/or function of the GPRK6 (G protein-coupled receptor kinase 6) gene or a GPRK6 protein, respectively. GPRK6 has a direct functional relationship for example with the β2-adrenergic receptor, the vasoactive intestinal polypeptide type-1 (VPAC1) receptor, and the calcitonin gene-related peptide (CGRP) receptor (Shetzline et al. 2002, J. Biol. Chem. 277: 25519-25526; Aiyar et al. 2000, Eur. J. Pharmacol. 403: 1-7), thus possibly also being involved in the regulation of mechanisms underlying angioedema pathogenesis. The GPRK6 gene is located ˜15 kb telomeric from the coagulation factor XII gene, being encoded on the opposite strand. There appear to exist certain splice variants/isoforms of GPRK6 (c.f. AceView and UCSC Genome Browser; GenBank acc nos. BX355118, BX463737, BI604127 [isoform h]) that arise from or are related to genomic sequences within the coagulation factor XII gene or its extended promoter region.

As stated above, factor XII (i.e. coagulation factor XII) is preferably a serine protease produced by the liver, circulating in human plasma as a single-chain inactive zymogen at a concentration of approximately 30 μg/ml. From expression data one has to assume a coagulation factor XII production also by other tissues, possibly as isoforms. Coagulation factor XII has a molecular weight of about 80 kDa on SDS gel electrophoresis and was originally cloned and sequenced by Cool et al. 1985 (J. Biol. Chem. 260: 13666-13676) and by Que & Davie 1986 (Biochemistry 25: 1525-1528). The human coagulation factor XII gene is located on chromosome 5, at 5q35.3 (Royle et al. 1988, Somat. Cell Mol. Genet. 14: 217-221), it is approximately 12 kb in size and consists of 14 exons and 13 introns (Cool & MacGillivray 1987, J. Biol. Chem. 262: 13662-13673). The mature plasma protein consists of 596 amino acids (following a leader peptide of 19 residues) and is organized in several domains. From N-terminus to C-terminus, these domains are: a fibronectin type-II domain, an epidermal growth factor-like domain, a fibronectin type-I domain, another epidermal growth factor-like domain, a kringle domain, a proline-rich region, and a serine-protease catalytic region.

In vitro activation of factor XII occurs on negatively charged surfaces (including glass, kaolin, Celite, dextran sulfate, and ellagic acid), by autoactivation, by proteolytic cleavage, by conformational change, or by some combination of these mechanisms (Pixley & Colman 1993, Methods Enzymol. 222: 51-65). Further activating substances include sulfatides, chondroitin sulfate, endotoxin, some mast cell proteoglycans, and also aggregated Aβ protein of Alzheimer's disease. In vivo, the subendothelial vascular basement membrane and/or the stimulated endothelial cell surface might be important for factor XII activation (Pixley & Colman 1993). On endothelial cell membranes, urokinase plasminogen activator receptor, gC1qR (the receptor that binds to the globular heads of complement C1q), and cytokeratin 1 might be involved in the interaction with factor XII (Joseph K. et al. 1996, Proc. Natl. Acad. Sci. USA 93: 8552-8557; Joseph K. et al. 2001, Thromb. Haemost. 85: 119-124; Mandi et al. 2002, Blood 99: 3585-3596).

Primary activation of factor XII is due to cleavage of the molecule at a critical Arg₃₅₃-Val₃₅₄ bond contained within a disulfide bridge, mediated for example by kallikrein or plasmin (or factor XIIa itself). The resultant factor XIIa (a-coagulation factor XIIa) is thus a two-chain, disulfide-linked 80-kDa enzyme consisting of a heavy chain (353 residues; 50 kDa) and a light chain (243 residues; 28 kDa). The heavy chain binds to negatively charged surfaces, the light chain represents the serine protease part of the molecule containing the canonical Asp₄₄₂, His₃₉₃, Ser₅₄₄ triad. Two subsequent cleavages are responsible for the formation of the two forms of factor XIIf (Kaplan et al. 2002, J. Allergy Clin. Immunol. 109: 195-209): these cleavages occur at Arg334-Asn335 and Arg343-Leu344 and result in the formation of “factor XII fragment”, FXIIf, also called β-FXIIa. FXIIf consists of the light chain of factor XIIa, corresponding to the serine protease domain, and a very small piece, either 19 or 9 amino acids in length, of the original heavy chain. Factor XIIf lacks the binding site for the activating surface as well as the ability of factor XIIa to convert factor XI to factor XIa. However, FXIIf is still a potent activator of prekallikrein. In summary, activation of the factor XII zymogen results in an enzyme with decreasing size, a decrease in surface-binding properties, and a decrease in coagulant activity, but retained, eventually increased kinin-forming capacity (Colman & Schmaier 1997, Blood 90: 3819-3843).

The present invention's disclosure allows to specifically identify individuals with a mutation in a nucleic acid molecule encoding coagulation factor XII or regulating the expression of coagulation factor XII and link this mutation with the individual's drug-associated angioedema or its predisposition to develop a drug-associated angioedema or to pass on to their offspring a specific allele which is associated with an increased risk for the development of drug-associated angioedema. Said nucleic acid molecule may be DNA or RNA.

Any method including those known to the person skilled in the art may be used to determine the presence or absence of such a mutation.

The determination of the presence or absence of a disease-associated mutation will be of great value, for example, as a predictive pharmacogenetic marker. Considering the fact that angioedema can be a life-threatening adverse drug reaction, it is desirable to have a test in hands which allows to identify individuals who carry an increased risk or a predisposition for the development of drug-associated angioedema. If it is intended, for example, to treat a patient suffering from hypertension with an ACE inhibitor, it would be desirable to know if a disease-associated mutation is present in this patient. If this is the case a particular type of medication can be chosen, namely a medication that is known not to be associated with a risk of developing angioedema. Thus, it will be possible to avoid exposing the patient to the risk of developing a treatment-associated angioedema attack.

Such a test will also be valuable with respect to a patient already being affected by a drug-associated angioedema. In such a case the recognition of the presence of a disease-associated mutation in a nucleic acid molecule regulating the expression of or encoding coagulation factor XII will allow to relate the presence of such a mutation to the occurrence of angioedema symptoms, and, thus, to choose for example an effective specific treatment, namely a treatment targeting the underlying cause.

Further, it is envisaged that therapeutic means and measures, disclosed in the present invention, can also be used for the purpose of prevention, for example in a situation in which a patient is positive for a disease-associated mutation but nevertheless, for certain medical reasons, is preferred to be treated or must be treated with a specific drug known to be associated with an increased risk for the development of angioedema.

In a preferred embodiment of the present invention's method of diagnosing, said determination comprises hybridizing under stringent conditions to said nucleic acid molecule at least one pair of nucleic acid probes, the first probe of said pair being complementary to the wild-type sequence of said nucleic acid molecule and the second probe of said pair being complementary to the mutant sequence of said nucleic acid molecule, wherein a perfect match, the presence of stable hybridization, between (i) the first hybridization probe and the target nucleic acid molecule indicates the presence of a wild-type sequence, and (ii) the second hybridization probe and the target nucleic acid molecule, indicates the presence of a mutant sequence, wherein the first hybridization probe and the second hybridization probe allow a differential detection. Preferably, said mutant sequence is a disease-associated mutant sequence.

The term “hybridizing under stringent conditions”, as used in the description of the present invention, is well known to the skilled artesian and corresponds to conditions of high stringency or selectivity. Appropriate stringent hybridization conditions for each sequence may be established by a person skilled in the art on well-known parameters such as temperature, composition of the nucleic acid molecules, salt conditions etc.; see, for example, Sambrook et al., “Molecular Cloning, A Laboratory Manual”; ISBN: 0879695765, CSH Press, Cold Spring Harbor, 2001, or Higgins and Hames (eds.), “Nucleic acid hybridization, a practical approach”, IRL Press, Oxford 1985, see in particular the chapter “Hybridization Strategy” by Britten & Davidson, 3 to 15. Stringent hybridization conditions are, for example, conditions comprising overnight incubation at 42° C. in a solution comprising: 50% formamide, 5×SSC (750 mM NaCl, 75 mM trisodium citrate), 50 mM sodium phosphate (pH 7.6), 5×Denhardt's solution, 10% dextran sulfate, and 20 micrograms/ml denatured, sheared salmon sperm DNA, followed by washing the filters in 0.1×SSC at about 65°. Other stringent hybridization conditions are for example 0.2×SSC (0.03 M NaCl, 0.003 M sodium citrate, pH 7) at 65° C.

Depending on the particular conditions, for example the base composition of the probe, the person skilled in the art may have to vary, for example the salt concentration and temperature in order to find conditions which (a) prevent the hybridization of probes differing from the target nucleic acid molecule in only one position and (b) still allow hybridization of probes which completely match the same region of the target nucleic acid molecule. However, said conditions can be established by standard procedures known to the person skilled in the art and by routine experimentation.

The probe of hybridization is usually a nucleic acid molecule containing one or more labels. The label can be located at the 5′ and/or 3′ end of the nucleic acid molecule or be located at an internal position. Preferred labels include, but are not limited to, fluorochromes, e.g. carboxyfluorescein (FAM) and 6-carboxy-X-rhodamine (ROX), fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may also be a two stage system, where the probe is conjugated to biotin, haptens, etc. having a high affinity binding partner, e.g. avidin, specific antibodies, etc., where the binding partner is conjugated to a detectable label.

As stated above, two probes used as a pair must allow a differential detection. This can be accomplished, for example, by labeling the probes with two different labels that can be differentiated in a detection process.

The hybridization probe is usually a nucleic acid molecule of about 20 to about 2000 bases in length. When used for hybridization reactions such as southern or northern blot reactions, the probe can be an oligonucleotide or primer which are typically in the range of about 15 to 50 bases in length or can be considerably longer and may range from about 50 bases to about 2000 bases. The term “oligonucleotide”, when used in an amplification reaction, refers to a nucleic acid molecule of typically 15 to 50 bases in length with sufficient complementarity to allow specific hybridization to a nucleic acid sequence encoding or regulating the expression of coagulation factor XII. Preferably, an oligonucleotide used for hybridization or amplification is about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases in length.

However, probes of about 100, 150, 200, 250, 300, 350, 400, 450, 500, 550, 600, 650, 700, 750, 800, 850, 900, 950 or 1000 bases are also contemplated by the present invention. Moreover, according to the particular conditions chosen for hybridization, the nucleotide probe may even be several hundred or thousand bases longer. Said probe or oligonucleotide may be composed of DNA or RNA. When used as a hybridization probe, it may be, e.g., desirable to use nucleic acid analogs, in order to improve the stability and binding affinity. The term “nucleic acid” shall be understood to encompass such analogs. A number of modifications have been described that alter the chemistry of the phosphodiester backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include, but are not limited to, 3′-O′-5′-S-phosphorothioate, 3′-S-5-O-phosphorothioate, 3′-CH₂-5′-O-phosphonate and 3′-NH-5-O-phosphoroamidate. Peptide nucleic acids replace the entire phosphodiester backbone with a peptide linkage. Sugar modifications are also used to enhance stability and affinity. The a-anomer of deoxyribose may be used, where the base is inverted with respect to the natural b-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine; 5-propynyl-2′-deoxyuridine and 5-propynyl-2′-deoxycytidine for deoxythymidine and deoxycytidine, respectively.

In another preferred embodiment of the present invention's method of diagnosing, said method comprises hybridizing under stringent conditions to said nucleic acid molecule a hybridization probe specific for a mutant sequence. Preferably, said mutant sequence is a disease-associated mutant sequence.

In another preferred embodiment of the present invention, the method of diagnosing comprises a step of nucleic acid amplification and/or nucleic acid sequencing. Preferably, nucleic acid sequencing is DNA sequencing. A widely used method of diagnosing is for example direct DNA sequencing of PCR products containing a mutation to be diagnosed. The term “amplification” or “amplify” means increase in copy number. The person skilled in the art know various methods to amplify nucleic acid molecules, these methods may also be used in the present invention's method of diagnosing. Amplification methods include, but are not limited to, “polymerase chain reaction” (PCR), “ligase chain reaction” (LCR, EPA320308), “cyclic probe reaction” (CPR), “strand displacement amplification” (SDA, Walker et al. 1992, Nucleic Acid Res. 7: 1691-1696), “transcription based amplification systems” (TAS, Kwoh et al. 1989, Proc. Nat. Acad. Sci. USA 86: 1173; Gingeras et al., PCT Application WO 88/10315). Preferably, amplification of DNA is accomplished by using polymerase chain reaction (PCR) [Methods in Molecular Biology, Vol. 226 (Bartlett J. M. S. & Stirling D., eds.): PCR protocols, 2^{nd} edition; PCR Technology: Principles and Applications for DNA Amplification (Erlich H. A., ed.), New York 1992; PCR Protocols: A guide to methods and applications (Innis M. A. et al., eds.), Academic Press, San Diego 1990]. Nucleic acid amplification methods may be particularly useful in cases when the sample contains only minute amounts of nucleic acid. If said nucleic acid is RNA, an RT-PCR might be performed. Subsequently, another amplification step involving PCR may be performed. Alternatively, if said nucleic acid contained in the sample is DNA, PCR may be performed.

The PCR, generally, consists of many repetitions of a cycle which consists of: (a) a denaturing step, which melts both strands of a DNA molecule; (b) an annealing step, which is aimed at allowing the primers to anneal specifically to the melted strands of the DNA molecule; and (c) an extension step, which elongates the annealed primers by using the information provided by the template strand. Generally, PCR can be performed for example in a 50 μl reaction mixture containing 5 μl of 10×PCR buffer with 1.5 mM MgCl₂, 200 μM of each deoxynucleoside triphosphate, 0.5 μl of each primer (10 μM), about 10 to 100 ng of template DNA and 1 to 2.5 units of Taq Polymerase. The primers for the amplification may be labeled or be unlabeled. DNA amplification can be performed, e.g., with a model 2400 thermal cycler (Applied Biosystems, Foster City, Calif.): 2 min at 94° C., followed by 35 cycles consisting of annealing (30 s at 50° C.), extension (1 min at 72° C.), denaturing (10 s at 94° C.) and a final annealing step at 55° C. for 1 min as well as a final extension step at 72° C. for 5 min. However, the person skilled in the art knows how to optimize these conditions for the amplification of specific nucleic acid molecules or to scale down or increase the volume of the reaction mix.

A further method of nucleic acid amplification is the “reverse transcriptase polymerase chain reaction” (RT-PCR). This method is used when the nucleic acid to be amplified consists of RNA. The term “reverse transcriptase” refers to an enzyme that catalyzes the polymerization of deoxyribonucleoside triphosphates to form primer extension products that are complementary to a ribonucleic acid template. The enzyme initiates synthesis at the 3′-end of the primer and proceeds toward the 5′-end of the template until synthesis terminates. Examples of suitable polymerizing agents that convert the RNA target sequence into a complementary, copy-DNA (cDNA) sequence are avian myeloblastosis virus reverse transcriptase and Thermus thermophilus DNA polymerase, a thermostable DNA polymerase with reverse transcriptase activity marketed by Perkin Elmer. Typically, the genomic RNA/cDNA duplex template is heat denatured during the first denaturation step after the initial reverse transcription step leaving the DNA strand available as an amplification template. Suitable polymerases for use with a DNA template include, for example, E. coli DNA polymerase I or its Klenow fragment, T.sub.4 DNA polymerase, Tth polymerase, and Taq polymerase, a heat-stable DNA polymerase isolated from Thermus aquaticus and developed and manufactured by Hoffmann-La Roche and commercially available from Perkin Elmer. The latter enzyme is widely used in the amplification and sequencing of nucleic acids. The reaction conditions for using Taq polymerase are known in the art and are described, e.g., in: PCR Technology, Erlich, H. A. 1989, Stockton Press, New York; or in: Innis, M. A., D. H. Gelfand, J. J. Sninsky, and T. J. White. 1990, PCR Protocols: A guide to methods and applications. Academic Press, New York. High-temperature RT provides greater primer specificity and improved efficiency. Copending U.S. patent application Ser. No. 07/746, 121, filed Aug. 15, 1991, describes a “homogeneous RT-PCR” in which the same primers and polymerase suffice for both the reverse transcription and the PCR amplification steps, and the reaction conditions are optimized so that both reactions occur without a change of reagents. Thermus thermophilus DNA polymerase, a thermostable DNA polymerase that can function as a reverse transcriptase, can be used for all primer extension steps, regardless of template. Both processes can be done without having to open the tube to change or add reagents; only the temperature profile is adjusted between the first cycle (RNA template) and the rest of the amplification cycles (DNA template). The RT Reaction can be performed, for example, in a 20 μl reaction mix containing: 4 μl of 5×ANV-RT buffer, 2 μl of Oligo dT (100 μg/ml), 2 μl of 10 mM dNTPs, 1 μl total RNA, 10 Units of AMV reverse transcriptase, and H₂O to 20 μl final volume. The reaction may be, for example, performed by using the following conditions: The reaction is held at 70 C.° for 15 minutes to allow for reverse transcription. The reaction temperature is then raised to 95 C.° for 1 minute to denature the RNA-cDNA duplex. Next, the reaction temperature undergoes two cycles of 95° C. for 15 seconds and 60 C.° for 20 seconds followed by 38 cycles of 90 C.° for 15 seconds and 60 C.° for 20 seconds. Finally, the reaction temperature is held at 60 C.° for 4 minutes for the final extension step, cooled to 15 C.°, and held at that temperature until further processing of the amplified sample.

The term “primer” or “oligonucleotide” refers to a short nucleic acid molecule from about 8 to about 30, eventually to about 50 nucleotides in length, whether natural or synthetic, capable of acting as a point of initiation of nucleic acid synthesis under conditions in which synthesis of a primer extension product complementary to a template nucleic acid strand is induced, i.e., in the presence of four different nucleoside triphosphates or analogues thereof and an agent for polymerisation (i.e., DNA polymerase or reverse transcriptase) in an appropriate buffer and at a suitable temperature. Preferably, a primer is a single-stranded oligodeoxyribonucleotide. The appropriate length of a primer depends on the intended use of the primer but typically ranges for PCR primers and primers used in sequencing reactions from 10 to 25 nucleotides. Short primer molecules generally require cooler temperatures to form sufficiently stable hybrid complexes with the template. A primer need not reflect the exact sequence of the template but must be sufficiently complementary to hybridize specifically with a template, provided its ability to mediate amplification is not compromised. “Hybridize” refers to the binding of two single stranded nucleic acids via complementary base pairing, i.e. A to T (in RNA: U), G to C. The term “primer pair” refers to two primers that hybridize with the + and − strand, respectively, of a double stranded nucleic acid molecule, and allow the amplification of e.g. DNA fragments, as for example in a PCR reaction. A primer can be labeled, if desired, by incorporating a compound detectable by spectroscopic, photochemical, biochemical, immunochemical, or chemical means. For example, useful labels include, but are not limited to, fluorescent dyes, electron-dense reagents, biotin, or small peptides for which antisera or monoclonal antibodies are available. A label can also be used to “capture” the primer, so as to facilitate a selection of amplified nucleic acid or fragments thereof. Carboxyfluorescein (FAM) and 6-carboxy-X-rhodamine (ROX) are preferred labels. However, other preferred labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may also be a two stage system, where the primer is conjugated to biotin, haptens, etc. having a high affinity binding partner, e.g. avidin, specific antibodies, etc., where the binding partner is conjugated to a detectable label. The label may be conjugated to one or both of the primers.

During said method for diagnosing, a step of nucleic acid sequencing may be performed. Any methods known in the art may be used for sequencing. Preferably, the nucleic acid sequence is determined by a method based on the sequencing techniques of Sanger or Maxam/Gilbert (see for example: Methods in Molecular Biology, Vol. 167 (Graham C. A. & Hill A. J. M., eds.): DNA sequencing protocols. 2^{nd} edition, 2001; Galas D. J. & McCormack S. J., Genomic Technologies: Present and Future. Caister Academic Press, Wymondham, UK, 2002).

In another preferred embodiment of the present invention's method of diagnosing, said method is or comprises an allele discrimination method selected from the group consisting of allele-specific hybridization, allele-specific primer extension including allele-specific PCR, allele-specific oligonucleotide ligation, allele-specific cleavage of a flap probe and/or allele-specific cleavage using a restriction endonuclease. These methods are known to the skilled person and described and further referenced for example by Kwok P-Y & Chen X 2003, Curr. Issues Mol. Biol. 5:43-60; Kwok P-Y 2001, Annu. Rev. Genomics Hum. Genet. 2:235-258; Syvanen, A.-Ch. 2001, Nature Rev. Genet. 2: 930-942.

In yet a further preferred embodiment, the present invention's method of diagnosing may comprise a detection method selected from the group consisting of fluorescence, time-resolved fluorescence, fluorescence resonance energy transfer (FRET), fluorescence polarization, colorimetric methods, mass spectrometry, (chemi)luminescence, electrophoretical detection and electrical detection methods. These methods for the detection of an allele discrimination reaction are known to the skilled person and described and further referenced for example by Kwok P-Y & Chen X 2003, Curr. Issues Mol. Biol. 5:43-60; Kwok P-Y 2001, Annu. Rev. Genomics Hum. Genet. 2:235-258; Syvanen, A.-Ch. 2001, Nature Rev. Genet. 2: 930-942.

In certain cases it might be necessary to detect large deletions, insertions, or duplications. Preferably, this may be done by using methods well known in the art and comprising, for example, Southern blotting methods; quantitative or semi-quantitative gene dosage methods including competitive PCR, differential PCR, real-time PCR, multiplex amplifiable probe hybridization; or long-range PCR (Armour et al. 2002, Human Mutation 20: 325-337).

It may often be desirable to obtain, from a single individual, an allelic diagnosis at several regions or positions of the nucleic acid molecule(s) encoding coagulation factor XII or regulating its expression. For this purpose, nucleic acid arrays may be useful, such as those described in: W0 95/11995.

Further, for some purposes it may be desirable to determine the presence of two or more mutations/variations as a haplotype, i.e. to determine which alleles from several mutant/variant positions occur together on one haplotype. This can be achieved by methods known in the art, for example by a segregation analysis within families, and also and preferably by methods allowing molecular haplotyping. For example, a double digest of a single PCR product, containing two mutant/variant positions, with two restriction endonucleases, each one of these two enzymes being able to differentiate the allelic situation at one of the two investigated positions, can yield such haplotype information from the fragment sizes obtained. However, numerous other methods are known to the person skilled in the art (see, for example: Tost et al. 2002, Nucleic Acids Res. 30: e96; Eitan & Kashi 2002, Nucleic Acids Res. 30: e62; Pettersson et al. 2003, Genomics 82: 390-396; Ding et al. 2003, Proc. Natl. Acad. Sci. U.S.A. 100: 7449-7453; Odeberg et al. 2002, Biotechniques 33: 1104, 1106, 1108; McDonald et al. 2002, Pharmacogenetics 12: 93-99; Woolley et al. 2000, Nature Biotechnol. 18: 760-763) and are envisaged to be applicable for the purposes of the present invention.

In yet another preferred embodiment of the present invention's method of diagnosing, the probe or the subject's nucleic acid molecule is attached to a solid support. Solid supports that may be employed in accordance with the invention include filter material, chips, wafers, microtiter plates, to name a few.

The present invention also relates to a method of diagnosing a drug-associated angioedema or a predisposition thereto in a subject being suspected of having developed or of having a predisposition to develop a drug-associated angioedema or in a subject being suspected of being a carrier for a drug-associated angioedema or in a subject being intended to be treated with a drug associated with the development of angioedema, the method comprising assessing the presence, amount and/or activity of coagulation factor XII in said subject and including the steps of: (a) determining from a biological sample of said subject in vitro, the presence, amount and/or activity of: (i) a (poly)peptide encoded by the coagulation factor XII gene; (ii) a substrate of the (poly)peptide of (i); or (iii) a (poly)peptide processed by the substrate mentioned in (ii); (b) comparing said presence, amount and/or activity with that determined from a reference sample; and (c) diagnosing, based on the difference between the samples compared in step (b), the pathological condition of a drug-associated angioedema or a predisposition thereto. The term “(poly)peptide” refers alternatively to peptide or to (poly)peptides. Peptides conventionally are covalently linked amino acids of up to 30 residues, whereas polypeptides (also referred to herein as “proteins”) comprise 31 and more amino acid residues. The term “assessing the amount” or “determining the amount” means assessing or determining the amount of a (poly)peptide encoded by the coagulation factor XII gene, comprising for example the coagulation factor XII precursor or any of its maturation products generated for example by activating processes including autoactivation and proteolytic processing of coagulation factor XII. Therefore, assessing or determining the amount of coagulation factor XII also may refer to determining the amount of (1) mature FXII, (2) FXIIa (80 kDa, arising from the cleavage at Arg353-Val354); (3) FXIIf (2 subforms: 30 kDa/28.5 kDa; 19-peptide or nonapeptide linked via S—S to the catalytic chain; arising from the cleavage of Arg334-Asn335 and the additional cleavage of Arg343-Leu344); (4) a third form of activated factor XII, a 40 kDa molecule (mainly produced by autoactivation), in which the serine protease domain is linked to a 12,000-MW fragment of the heavy chain (Kaplan & Silverberg 1987); (5) potential protein isoforms (AceView, http://www.ncbi.nlm.nih.gov/IEB/Research/Acembly/ay.cgi?db=33&c=Gene&I═F12); (6) coagulation factor XII forms or fragments that arise from an irregular proteolytic processing, eventually caused by a mutation of the present invention; or (7) a mutant of any one of the forms (1) to (5), including any of the mutants of the present invention. However, “assessing the amount” or “determining the amount” also refers to determining the amount of substrates and/or their activation products of any of the above-mentioned coagulation factor XII forms. Preferably, the ratio of activated and native (non-activated) forms of these substrates is determined. Also included are (poly)peptides processed by these (activated) substrates. These substrates and processed (poly)peptides include, for example, (8) coagulation factor XIa/coagulation factor XI; (9) coagulation factor Vila/coagulation factor VII; (10) kallikrein/prekallikrein; (11) plasmin/plasminogen; (12) activated complement C1r/C1r; (13) activated complement C1s/C1s; (14) activated hepatocyte growth factor (HGF)/hepatocyte growth factor; (15) activated macrophage stimulating protein (MSP)/macrophage stimulating protein. Also included is (16) the determination of “cleavage products of high-molecular weight kininogen” or the ratio of the “cleavage products of high-molecular weight kininogen” with “high-molecular weight kininogen”. Said cleavage products comprise cleaved kininogen, bradykinin and/or other kinins. Furthermore included are (17) cleavage products of complement component C2/complement component C2; (18) cleavage products of complement component C4/complement component C4; and (19) activated bradykinin type 2 receptor/bradykinin type 2 receptor. The term “assessing the activity” or “determining the activity” means determining a biological activity, wherein biological activity refers to (a) the known activities, preferably those of wild-type (poly)peptides, and (b) aberrant activities, including those of mutant coagulation factor XII (poly)peptides which are apparent from comparing the activity of a mutant with that of a wild-type (poly)peptide. The known and aberrant activities may comprise the activity of any of the proteins (1) to (19) mentioned above.

The term “assessing the presence” or “determining the presence” means determining which of the aforementioned (poly)peptides or proteins is present in the sample. Said term also refers to determining whether wild-type or a mutant (poly)peptide is present in the sample. Preferably, said (poly)peptide is any of the (poly)peptides (1) to (7) as mentioned above. In some cases, it may also be useful to analyze any of the (poly)peptides (8) to (19) as mentioned above, their native and/or activated forms. Step (i) of the method, which reads “a (poly)peptide encoded by the coagulation factor XII gene”, may comprise the determination of at least one of the (poly)peptides listed above under (1), (2), (3), (4), (5), (6) and (7).

Step (ii) of the method, which reads “a substrate of the (poly)peptide of (i)”, may comprise the determination of at least one of the polypeptides listed above under (8), (9), (10), (11), (12), (13), (14), (15) and (16).

Step (iii) of the method, which reads “a (poly)peptide processed by the substrate mentioned in (ii)”, may comprise the determination of at least one of the polypeptides listed above under (16), (17), (18), and (19).

This method of diagnosing is based on determining from a sample of an individual to be diagnosed and a reference sample the quantity and/or quality of any of the proteins listed under (1) to (19) and determining, based on the difference between said samples, a pathological condition or a predisposition thereto in said individual's sample. Said pathological condition is a drug-associated angioedema, preferably a coagulation factor XII-related drug-associated angioedema.

The reference sample is a standard sample obtained from a healthy subject or healthy subjects, preferably from a subject or subjects not affected by drug-associated angioedema and presumably not having a predisposition for drug-associated angioedema.

Generally, any of the known protein detection methods may be used. These include, for example, immunochemical, antibody-based methods such as ELISA, RIA, Western Blotting, preferably following any kind of electrophoretic separation step, and the like. Such methods are, for example, described by Clark & Hales: Immunoassays. In: Clinical Aspects of Immunology (P. J. Lachmann et al., eds.), vol. 2, 5^{th} ed., Boston 1993; or in Weir's Handbook of Experimental Immunology, 5^{th} ed., 1996 (Herzenberg L. et al., eds.); see also e.g. Lämmle et al. 1987 (Semin. Thromb. Hemost. 13: 106-114). Methods for the determination of biological activities of the polypeptides listed above are known in the art. Biological activity can be measured for example by providing substrates for the (poly)peptides and measuring substrate conversion by the methods known in the art. For example, measuring the activity of (pre)kallikrein on a chromogenic substrate, which may be monitored by detecting cleavage of said substrate, has been described by Kluft 1978 (J. Lab. Clin. Med, 91:83-95), Kluft 1988 (Meth. Enzymol. 163: 170-179). Functional assays for measuring prekallikrein have also been described by de la Cadena et al. 1987 (J. Lab. Clin. Med. 109: 601-607) and Silverberg & Kaplan 1988 (Meth. Enzymol. 163: 85-95). A functional assay for high molecular weight kininogen using a chromogenic substrate has been described by Scott et al. 1987 (Thromb. Res. 48: 685-700) and also by Gallimore et al. 2002 (Blood Coagul. Fibrinolysis 13: 561-568).

The present invention also employs methods for determining the amino acid sequence of a (poly)peptide. Such methods are known in the art (see for example: Methods in Molecular Biology, Vol. 211 (Smith B. J., ed.): Protein Sequencing Protocols. 2^{nd} edition, 2002). Preferably, protein sequence analysis is performed by Edman degradation (P. Edman, Acta Chem. Scand. 4: 283 (1950)) or by Matrix-assisted laser desorption/ionisation-time of flight mass spectrometry (MALDI-TOF MS). Hence, by using amino acid sequence analysis, the skilled person may determine whether a wild-type or mutant coagulation factor XII (poly)peptide is present in a sample.

The proteins listed above, include on the one hand coagulation factor XII and its various forms. These are part of a cascade known as, for example, the intrinsic coagulation pathway or contact system or kinin-forming pathway (see e.g. Kaplan et al. 1997, Adv. Immunol. 66: 225-272; Kaplan et al. 2002, J. Allergy Clin. Immunol. 109: 195-209). On the other hand, proteins listed above are proteins which follow coagulation factor XII downstream in said cascade, and, in addition, proteins which are not directly related to the kinin-forming pathway but for which it has been shown that they can be activated by coagulation factor XII, eventually indirectly. It is important to note that mutations of coagulation factor XII may have an impact on these downstream steps in the cascade and, for example, can result in a quantitatively or qualitatively abnormal activation of (poly)peptides located downstream in the cascade. This effect may be measured and may allow for deductions on the nature of the specific coagulation factor XII expressed in the individual under study.

The methods of the present invention are not limited to measuring individual (poly)peptides as listed above, but also refer to the measuring or determination of complexes of said (poly)peptides. Such complexes are for example complexes consisting of activated factor XII and complement C1 inhibitor; or complexes consisting of kallikrein and complement C1 inhibitor; or complexes consisting of kallikrein and alpha2-macroglobulin. Such complexes can be detected, for example, by using ELISA or RIA based techniques (Nuijens et al., 1987 Thromb. Hemost. 58: 778-785; Kaplan et al., 1985, Blood 66: 636-641; Kaplan et al., 1989, Clin. Immunol. Immunopathol. 50: S41-S51; Dors et al. 1992, Thromb. Haemost. 67: 644-648).

In a preferred embodiment of the present invention's method, the biological sample consists of or is taken from hair, skin, mucosal surfaces, body fluids, including blood, plasma, serum, urine, saliva, sputum, tears, liquor cerebrospinalis, semen, synovial fluid, amniotic fluid, milk, lymph, pulmonary sputum, bronchial secretion, or stool.

The term “biological sample” relates to the specimen taken from a mammal. Preferably, said specimen is taken from hair, skin, mucosal surfaces, body fluids, including blood, plasma, serum, urine, saliva, sputum, tears, liquor cerebrospinalis, semen, synovial fluid, amniotic fluid, milk, lymph, pulmonary sputum, bronchial secretion, or stool. However, it is important to note that many other samples might be useful for this purpose, for example a sample taken for histological or cytological purposes.

A variety of techniques for extracting nucleic acids from biological samples are known in the art. For example, see those described in Rotbart et al., 1989, in PCR Technology (Erlich ed., Stockton Press, New York) and Han et al. 1987, Biochemistry 26:1617-1625. If the sample is fairly readily disruptable, the nucleic acid need not be purified prior to amplification by the PCR technique, i.e., if the sample is comprised of cells, e.g. peripheral blood lymphocytes or monocytes, lysis and dispersion of the intracellular components may be accomplished merely by suspending the cells in hypotonic buffer. Suitable methods will vary depending on the type of specimen and are well known to the person skilled in the art (see e.g. Sambrook et al., “Molecular Cloning, A Laboratory Manual”; ISBN: 0879695765, CSH Press, Cold Spring Harbor, 2001).

It is apparent that, for analysis of mRNA, cDNA, or protein, the sample must be obtained from a tissue in which coagulation factor XII/the coagulation factor XII gene is expressed, or, respectively, from a tissue or body fluid, in which coagulation factor XII is expressed or in which it is secreted.

In another preferred embodiment, said presence, amount and/or activity is determined by using an antibody or an aptamer, wherein the antibody or aptamer is specific for (a) a (poly)peptide encoded by the coagulation factor XII gene, (b) a substrate of the (poly)peptide of (a), or (c) a (poly)peptide processed by the substrate mentioned in (b). The term “antibody” refers to monoclonal antibodies, polyclonal antibodies, chimeric antibodies, single chain antibodies, or a fragment thereof. Preferably the antibody is specific for a polypeptide listed under (1) to (19). The antibodies may be bispecific antibodies, humanized antibodies, synthetic antibodies, antibody fragments, such as Fab, F(ab₂)′, Fv or scFv fragments etc., or a chemically modified derivative of any of these, all comprised by the term “antibody”. Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals with modifications developed by the art. Furthermore, antibodies or fragments thereof to the aforementioned (poly)peptides can be obtained by using methods which are described, e.g., in Harlow and Lane “Antibodies, A Laboratory Manual”, CSH Press, Cold Spring Harbor, 1998. When derivatives of said antibodies are obtained by the phage display technique, surface plasmon resonance as employed in the BIAcore system can be used to increase the efficiency of phage antibodies S which bind to an epitope of the peptide or polypeptide to be analyzed (Schier, Human Antibodies Hybridomas 7 (1996), 97-105; Malmborg, J. Immunol. Methods 183 (1995), 7-13). The production of chimeric antibodies is described, for example, in WO89/09622.

Antibodies may be labelled. Preferably said label is selected from the group consisting of fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may also be a two stage system, where the antibody is conjugated to biotin, haptens, etc. having a high affinity binding partner, e.g. avidin, specific antibodies, etc., where the binding partner is conjugated to a detectable label. In another preferred embodiment of the present invention the label is a toxin, radioisotope, or fluorescent label.

The term “aptamers” refers to RNA and also DNA molecules capable of binding target proteins with high specificity, comparable with the specificity of antibodies. Methods for obtaining or identifying aptamers specific for a desired target are known in the art. Preferably, these methods may be based on the “systematic evolution of ligands by exponential enrichment” (SELEX) process (Ellington and Szostak, Nature, 1990, 346: 818-822; Tuerk and Gold, 1990, Science 249: 505-510; Fitzwater & Polisky, 1996, Methods Enzymol. 267: 275-301). Preferably, said aptamers may be specific for any of the (poly)peptides listed under (1) to (19). The use of aptamers for detection and quantification of polypeptide targets is described in, for example, McCauley et al., 2003, Anal. Biochem., 319:244-250; Jayasena, 1999, Clin. Chem. 45:1628-1650.

In a more preferred embodiment, said antibody or aptamer is specific for a (poly)peptide encoded by the coagulation factor XII gene. Said reagents will allow for assessing the quantity and/or quality of (a) coagulation factor XII (poly)peptide(s), and eventually also for the differentiation between wild-type and mutant, preferably disease-associated mutant, coagulation factor XII (poly)peptides. For example, the identification of coagulation factor XII (poly)peptides by an immunoblotting procedure following an electrophoretic separation step, might well allow for the recognition of a mutant coagulation factor XII (poly)peptide. However, regarding the preferred differentiation between wild-type and disease-associated mutant coagulation factor XII (poly)peptides, preferably, said antibody or aptamer is specific for a disease-associated mutant of the present invention. Such an antibody or aptamer would fail to bind to wild-type coagulation factor XII (poly)peptide(s) but bind to a disease-associated mutant with high specificity. This antibody or aptamer would therefore be most useful to discriminate between wild-type and mutant coagulation factor XII (poly)peptides. More preferably, the epitope or target region recognized by the antibody or aptamer comprises the mutant position/region in coagulation factor XII.

Various antibody-based methods for the determination of coagulation factor XII (poly)peptide(s), like radial immunodiffusion, electroimmunoassay according to Laurell, dot immunobinding assay, radioimmunoassay, enzyme immunoassay, enzyme-linked immunosorbent assay, immunoblotting, or alike, have been described or employed for example by Mannhalter et al. 1987 (Fibrinolysis 1: 259-263), Gevers Leuven et al. 1987 (J. Lab. Clin. Med.), Wuillemin et al. 1990 (J. Immunol. Methods 130: 133-140), Saito et al. 1976 (J. Lab. Clin. Med. 88: 506-514), Ford et al. 1996 (J. Immunoassay 17: 119-131), Lämmle et al. 1987 (Semin. Thromb. Hemost. 13: 106-114).

In a preferred embodiment of the present invention, the presence, amount and/or activity of the (poly)peptide(s) encoded by the coagulation factor XII gene is determined in (a) a coagulation assay; or in (b) a functional amidolytic assay; or in (c) a mitogenic assay; or in (d) a binding assay measuring binding of a (poly)peptide encoded by the coagulation factor XII gene to a binding partner.

Coagulant activity of coagulation factor XII may be quantified using methods in which correction of the abnormal clotting time, the prolonged activated partial thromboplastin time, of plasma of a person with a severe hereditary deficiency of coagulation factor XII is measured (see for example: Pixley R. A. & Colman R. W. 1993; Methods in Enzymology 222: 51-65). Functional amidolytic assays for coagulation factor XII using various synthetic chromogenic substrates (for example S2302, S2337, S2222) have been described for example by Vinazzer 1979 (Thrombosis Research 14: 155-166), Tans et al. 1987 (Eur. J. Biochem. 164: 637-642), Gallimore et al. 1987 (Fibrinolysis 1: 123-127), Walshe et al. 1987 (Thrombosis Research 47: 365-371), Kluft 1988 (Methods Enzymol. 163: 170-179), Stürzebecher et al. 1989 (Thrombosis Research 55: 709-715).

Another example for assessing a coagulation factor XII functional activity may be a measurement of the hepatocyte growth factor activating activity of coagulation factor XII (Shimomura et al. 1995, Eur. J. Biochem. 229: 257-261).

Schmeidler-Sapiro et al. 1991 (Proc. Natl. Acad. Sci. U.S.A. 88: 4382-4385) described assay systems allowing to assess a mitogenic activity of coagulation factor XII on HepG2 cells; coagulation factor XII as well as coagulation factor XIIa (kaolin-activated coagulation factor XII) enhanced cell proliferation and thymidine and leucine incorporation in HepG2 cells. Gordon et al. 1996 (Proc. Natl. Acad. Sci. U.S.A. 93: 2174-2179) assessed a growth factor activity of factor XII on several other target cells. Any of the aforementioned methods may be modified and used for determining the activity of (poly)peptides encoded by the coagulation factor XII gene. Various activators can be used in these assays, for example dextran sulfate, kaolin, a cephalin ellagic acid based reagent (Walshe et al. 1987, Thromb. Res. 47: 365-371), or others, and it is conceivable that the extent and/or the nature of activation achieved could be different for disease-associated mutant forms of coagulation factor XII when compared to wild-type coagulation factor XII (poly)peptide(s).

The term “binding partner” refers to a molecule capable of interacting with a (poly)peptide encoded by the coagulation factor XII gene. The binding activity of coagulation factor XII (poly)peptides may be determined by using a binding assay. The skilled person knows from in vitro studies that coagulation factor XII may bind for example to activating surfaces or substances, proteins or protein complexes. The prior art reported for example about the binding of coagulation factor XII to complexes of gC1q-R, cytokeratin 1 and urokinase plasminogen activator receptor present on the surface of endothelial cells (Joseph et al. 1996, Proc. Natl. Acad. Sci. USA 93: 8552-8557; Joseph et al. 2001, Thromb. Haemost. 85: 119-124; Mandi et al. 2002, Blood 99: 3585-3596). The binding partner can also be an antibody. Binding assays are described in detail in the prior art and may be used by the skilled person in order to determine whether a sample contains coagulation factor XII (poly)peptide(s) with normal or aberrant binding characteristics. This will allow deductions on the nature of the coagulation factor XII (poly)peptide(s) present in the sample under study.

The present invention also relates to a method of identifying a compound modulating coagulation factor XII activity which is suitable as a medicament or a lead compound for a medicament for the treatment and/or prevention of drug-associated angioedema, the method comprising the steps of: (a) in vitro contacting a coagulation factor XII (poly)peptide or a functionally related (poly)peptide with the potential modulator; and (b) testing for modulation of coagulation factor XII activity, wherein modulation of coagulation factor XII activity is indicative of a compound's suitability as a medicament or a lead compound for a medicament for the treatment and/or prevention of drug-associated angioedema.

The term “modulator” or “modulating compound” refers to a compound which alters the activity and/or the expression and/or the secretion of coagulation factor XII. This includes also the modulation of a “functionally related (poly)peptide”, thus of (a) (poly)peptide(s) or the expression thereof being related to the function and/or expression and/or secretion of coagulation factor XII, preferably functionally related to coagulation factor XII upstream or downstream within the contact system/kinin pathway. In principle, a modulator can have an activating or an inhibiting effect. It is also envisaged that the modulator can differentially modulate only one or more of the various functions of coagulation factor XII. The modulator can be, for example, a ‘small molecule’, an aptamer, or an antibody (see below). The condition to be treated or to be prevented due to said modulator is a drug-associated angioedema, preferably a drug-associated angioedema that is linked to an abnormal coagulation factor XII function and/or expression and/or secretion. In accordance with the present invention, the modulator is preferably a compound interacting with a coagulation factor XII (poly)peptide, and, more preferably, an inhibiting compound.

The term “contacting” means bringing in contact the targeted (poly)peptide, preferably a coagulation factor XII (poly)peptide with a potential modulator. Said coagulation factor XII (poly)peptide is preferably a polypeptide selected from any of the aforementioned (poly)peptides (1) to (7). By bringing in contact the (poly)peptide with a potential modulator of activity, the skilled person can test the impact of the modulator on the (poly)peptide's activity. Examples for assays for measuring various activities of coagulation factor XII (poly)peptides, including the binding to activating substances or other binding partners, have been described above and can be used for testing of potential modulators.

Coagulation factor XII (poly)peptide(s) used for contacting with a potential modulator may generate from various sources. For example, coagulation factor XII (poly)peptide(s) may be isolated from human plasma; to this end, various methods known in the art may be used, for example those described by Pixley & Colman 1993 (Methods Enzymol. 222: 51-65). Alternatively, coagulation factor XII (poly)peptide(s) may also be produced synthetically. Further, coagulation factor XII (poly)peptide(s) may be recombinantly expressed. To this end, nucleic acid molecules encoding coagulation factor XII (poly)peptides may be introduced into a host cell. The term “introducing” refers to the process of transfecting or transforming a host cell with such a nucleic acid molecule. Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986). Said nucleic acid molecule introduced into the host cell comprises an open reading frame encoding a coagulation factor XII (poly)peptide in expressable form. A typical mammalian expression vector contains the promoter element, which mediates the initiation of transcription of mRNA, the protein coding sequence, and signals required for the termination of transcription and polyadenylation of the transcript. Additional elements might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells. Alternatively, the recombinant (poly)peptide can be expressed in stable cell lines that contain the gene construct integrated into a chromosome. The co-transfection with a selectable marker such as dhfr, gpt, neomycin, hygromycin allows the identification and isolation of the transfected cells. The transfected nucleic acid can also be amplified to express large amounts of the encoded (poly)peptide. The DHFR (dihydrofolate reductase) marker is useful to develop cell lines that carry several hundred or even several thousand copies of the gene of interest. Another useful selection marker is the enzyme glutamine synthase (GS) (Murphy et al. 1991, Biochem J. 227:277-279; Bebbington et al. 1992, Bio/Technology 10:169-175). Using these markers, the mammalian cells are grown in selective medium and the cells with the highest resistance are selected. Chinese hamster ovary (CHO) and NSO cells are often used for the production of proteins. The expression vectors pC1 and pC4 contain the strong promoter (LTR) of the Rous Sarcoma Virus (Cullen et al. 1985, Molecular and Cellular Biology 5: 438-447) plus a fragment of the CMV-enhancer (Boshart et al. 1985, Cell 41:521-530). Multiple cloning sites, e.g., with the restriction enzyme cleavage sites Barn HI, Xba I and Asp 718, facilitate the cloning of the gene of interest. The vectors contain in addition the 3′ intron, the polyadenylation and termination signal of the rat preproinsulin gene. As indicated above, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

The recombinantly expressed polypeptide may contain additional amino acid residues in order to increase the stability or to modify the targeting of the protein. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to stabilize and purify proteins. For example, EP-A-0 464 533 (Canadian counterpart 2045869) discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0 232 262). On the other hand, for some uses it would be desirable to be able to delete the Fc part after the fusion protein has been expressed, detected and purified in the advantageous manner described. This is the case when the Fc portion proves to be a hindrance for example for the catalytic activity of a coagulation factor XII (poly)peptide. In drug discovery, for example, human proteins, such as hIL-5, have been fused with Fc portions for the purpose of high-throughput screening assays to identify antagonists of hIL-5. See, D. Bennett et al., J. Molecular Recognition 8:52-58 (1995) and K. Johanson et al., J. Biol. Chem. 270:9459-9471 (1995). Coagulation factor XII (poly)peptide(s) can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography and/or hydroxylapatite chromatography. Most preferably, high performance liquid chromatography (“HPLC”) is employed for purification.

The step of contacting the recovered coagulation factor XII (poly)peptide with a potential modulator is essentially a step by which the efficacy of a potential modulator is tested. Generally, the coagulation factor XII (poly)peptide is present at conditions assumed to be physiological conditions or in a test solution representing such conditions. When examining, for example, enzymatic activity, the following may be of importance: after optimum substrate and enzyme concentrations are determined, a candidate modulator is added to the reaction mixture at a range of concentrations. The assay conditions ideally should resemble the conditions under which the modulator is to be active, i.e., under physiologic pH, temperature, ionic strength, etc. For example, when the modulator is an inhibitor of protease activity, suitable inhibitors will exhibit strong protease inhibition at concentrations which do not raise toxic side effects in the subject. Inhibitors which compete for binding to the protease's active site may require concentrations equal to or greater than the substrate concentration, while inhibitors capable of binding irreversibly to the protease's active site may be added in concentrations in the order of the enzyme concentration. Substrate conversion, i.e. proteolytic cleavage is conveniently measured by using labelled substrates such as labelled peptides representing the cleavage site of a natural substrate of coagulation factor XII.

One of the more popular protease detection methods is the use of fluorescence resonance energy transfer between a donor fluorophore at one end of the peptide chain, and a quencher at the other end of the peptide chain. These methods were reviewed by Knight “Fluorimetric assays of proteolytic enzymes,” Methods in Enzymol. (1995) 248:18-34, the contents of which are incorporated herein by reference. Here, proteolytic cleavage of the peptide link connecting the fluorophore and quencher liberates the quencher to diffuse away from the fluorophore. This results in an increase in fluorescence. A variation on this quencher method is taught by U.S. Pat. Nos. 5,605,809 and 6,037,137. This variation brings a first fluorophore in close proximity to a second fluorophore via a folded peptide backbone. This technique has the advantage that the protease cleavage site need not be immediately adjacent to either of the fluorophores. However it has the disadvantage that to avoid disrupting the folded structure, the length of the protease cleavage site should ideally fall between 2-15 amino acid residues in length. Another very popular method is the use of peptide-quenched fluorescent moieties, such as the 7-amino-4-methylcoumarin (AMC) fluorophore, the 7-amino-4-carbamoylmethylcoumarin fluorophore (Harris, et. al. PNAS 97: 7754-7759 (2000)), or the peptide quenched Rhodamine 110 fluorophore (Mange) et. al., U.S. Pat. No. 4,557,862). Here the intrinsic fluorescence of a fluorophore is quenched by one or more covalently linked peptides, and the fluorescence is restored upon cleavage of the peptide. Although the Rhodamine 110 molecule operates with high efficiency, uses visible light for excitation and emission, and is otherwise an excellent label for fluorescence based protease assays, it has a few drawbacks that limit its use. The Rhodamine 110 molecule is divalent and normally incorporates two peptides of identical sequence, with both “N” terminal peptide groups exposed. This has the drawback that peptides with this polarity can not be incorporated into the interior of a larger peptide chain. Thus this label has primarily been used for protease substrate assays where the Rhodamine 110 molecule effectively represents the final “C” terminal group on the substrate. Variations on Rhodamine 110 molecule methods, suitable for caspase assays, are taught by U.S. Pat. No. 6,248,904.

The test for protease activity of coagulation factor XII (poly)peptides may be performed in solution or with the coagulation factor XII (poly)peptide or the substrate or the modulator arrayed on a solid support, e.g. a microtiter plate. Microarray methods have become widely used for pharmaceutical and biochemical research, and a large number of microarrays are commercially available. Use of peptide microarrays, constructed by photochemical methods, for antibody recognition of peptide patterns was taught by Fodor et. al. 1991, Science 251: 767-773. Use of peptide microarrays for protein kinase or protein-protein binding was taught by MacBeath and Schreiber 2000, Science 289: 1760-1763. Here glass slides were chemically activated to covalently bind peptides, and various peptides were spotted onto the slides using conventional spotting equipment. The peptides formed a covalent bond with the derivatized glass. Alternative methods to attach peptides to solid supports are taught by U.S. Pat. No. 6,150,153, which teaches the use of polyethyleneimine layers to facilitate peptide linkages. U.S. Pat. No. 4,762,881 teaches the use of incorporating an artificial benzoylphenylalanine into a peptide and allowing the peptide to attach to a solid substrate having an active hydrogen (such as polystyrene) using ultraviolet light. U.S. Pat. No. 4,681,870 teaches methods for derivatizing silica surfaces to introduce amino or carboxyl groups, and then coupling proteins to these groups. U.S. Pat. Nos. 5,527,681 and 5,679,773 teach methods for immobilized polymer synthesis and display suitable for microarrays, and various fluorescent-labeling methods to detect proteolytic cleavage.

For protease substrate microarrays, the peptides on the microarray will further contain detection moieties (fluorescent tags, fluorescent quenchers, etc.) to generate a detectable signal corresponding to the level of proteolytic cleavage of the particular peptide zone in question. The peptides are bound to the surface of the solid support (either covalently or non-covalently) to the extent sufficient to prevent diffusion of the bound peptides upon application of liquid sample, and subsequent digestion and processing steps. In use, the completed microarray is exposed to a liquid sample, which contains a coagulation factor XII (poly)peptide under study. The sample will typically be covered with an optional cover to help distribute the sample evenly over the array, and to prevent evaporation. Typically the cover will be of a transparent flat material, such as a glass or plastic cover slip, to enable observation of the peptide zones during the course of the digestion reaction. During the protease digestion reaction, peptides with differential sequences or different modifications will typically be digested to a differential amount. The detectable signal generated by the detection moieties attached to each peptide region will be interrogated, typically at multiple time points during the digestion reaction. This conveys information as to the relative proteolytic activity of the studied coagulation factor XII (poly)peptide in the presence of a potential protease modulator or inhibitor, thus providing information on the suitability of the modulator for modulating, eventually inhibiting coagulation factor XII activity. Optionally, at the end of the reaction, a non-specific protease or a non-specific labeled moiety reacting agent may be added to the microarray to serve as a positive or negative control.

In a preferred embodiment of the present invention's method of identifying a modulator compound, the coagulation factor XII (poly)peptide of step (a) is present in cell culture or cell culture supernatant or in a subject's sample or purified from any of these sources. The cell culture could be for example a cell culture in which a coagulation factor XII (poly)peptide is recombinantly expressed or a culture of cells, for example hepatocytes, and preferably of human origin, that naturally express coagulation factor XII. The subject's sample could be, for example, blood plasma.

In another preferred embodiment of the present invention's method of identifying a modulator compound, said testing is performed by assessing the physical interaction between a coagulation factor XII (poly)peptide and the modulator and/or the effect of the modulator on the function of said coagulation factor XII (poly)peptide.

The person skilled in the art knows of various methods for detecting the interaction between a protein and a potential binding partner or modulator. One such method, for example, may be based on the testing of potential binding partners which are spotted onto a solid support. If bound to a solid support, incubation of said potential binding partners with a solution containing, for example, coagulation factor XII (poly)peptide might identify positions on the solid support, occupied with candidate binding partners. Binding of, for example, coagulation factor XII (poly)peptide(s) to said binding partner may be detected by various methods known in the art. For example, binding of coagulation factor XII to a binding partner could be visualized by incubating the solid support with a labeled antibody specific for coagulation factor XII. Preferred methods comprise biacore based detection methods, ELISA based methods.

It is also envisaged here, that the (poly)peptide targeted by the potential modulator can be—instead of a coagulation factor XII (poly)peptide—a (poly)peptide functionally related, upstream or downstream within the contact system, with coagulation factor XII, i.e. interacting with coagulation factor XII. Nevertheless, as further envisaged here, this may cause a modulation of coagulation factor XII activity. A modulator may be based on known compounds which may also be modified in order to adapt the compound to the requirements of the specific (poly)peptide to be targeted. The modulator can be, for example, a small molecule, an aptamer, or an antibody (vide infra).

Preferably, the modulator is a small molecule or small molecular compound and may be selected by screening a library of small molecules (“small molecule library”). The term “small molecule” or “small molecular compound” refers to a compound having a relative molecular weight of not more than 1000 D and preferably of not more than 500 D. It can be of organic or anorganic nature. A large number of small molecule libraries, which are commercially available, are known in the art. Thus, for example, a modulator may be any of the compounds contained in such a library or a modified compound derived from a compound contained in such a library. Preferably, such a modulator binds to the targeted (poly)peptide encoded by the coagulation factor XII gene with sufficient specificity, wherein sufficient specificity means preferably a dissociation constant (Kd) of less than 500 nM, more preferable less than 200 nM, still more preferable less than 50 nM, even more preferable less than 10 nM and most preferable less than 1 nM.

It is also envisaged to design small molecular compounds using so called molecular modeling methods. Small molecular compounds can be for example peptide derived. Preferred are compounds which mimic the transition state of substrates of coagulation factor XII. Suitable compounds would, for example, be peptide-derived substrates which do not contain a cleavable peptide bond. Preferably, such compounds contain a cleavage site of a natural substrate of coagulation factor XII, wherein the peptide bond between P1 and P1′ is replaced by a non-cleavable bond.

The peptide-based compounds and others, like compounds based on heterocyclic structures, may be for example known inhibitors of serine proteases or new compounds or compounds derived from preexisting inhibitors by derivatization. Preferably, such compounds are designed by computer modeling, wherein computer modeling means using virtual-screening tools for the search of compounds that bind, for example, to the substrate binding site of coagulation factor XII by using homology-modeling tools. Generally, these methods rely on the three-dimensional structure of proteins, preferably of proteins crystallized together with a substrate. More preferably, the substrate is replaced with a candidate modulator or inhibitor.

The design of molecules with particular structural relationships to part of a protein molecule like coagulation factor XII is well established and described in the literature (see for example Cochran 2000, Chem. Biol. 7, 85-94; Grzybowski et al. 2002, Acc. Chem. Res. 35, 261-269; Velasquez-Campoy et al. 2001, Arch. Biochem. Biophys. 380, 169-175; D'Aquino et al. 2000, Proteins: Struc. Func. Genet. Suppl. 4, 93-107.). Any of these so-called “molecular modeling” methods for rational drug design can be used to find a modulator of coagulation factor XII. Most of these molecular modeling methods take into consideration the shape, charge distribution and the distribution of hydrophobic groups, ionic groups and hydrogen bonds in the site of interest of the protein molecule. Using this information, that can be derived e.g. from the crystal structure of proteins and protein-substrate complexes, these methods either suggest improvements to existing proposed molecules, construct new molecules on their own that are expected to have good binding affinity, screen through virtual compound libraries for such molecules, or otherwise support the interactive design of new drug compounds in silico. Programs such as GOLD (G. Jones, et al., Development and J. Mol. Biol., 267, 727-748 (1997)); FLEXR (B. Kramer et al., Structure, Functions, and Genetics, Vol. 37, pp. 228-241, 1999); FLEXE (M. Rarey et al., JMB, 261, 470-489 (1996)) DOCK (Kuntz, I. D. Science 257: 1078-1082, 1992); AUTODOCK (Morris et al., (1998), J. Computational Chemistry, 19: 1639-1662) are virtual screening programs designed to calculate the binding position and conformation as well as the corresponding binding energy of an organic compound to a protein. These programs are specially trimmed to allow a great number of “dockings”, that is calculations of the conformation with the highest binding energy of a compound to a binding site, per time unit. Their binding energy is not always a real value, but can be statistically related to a real binding energy through a validation procedure. These methods lead to molecules, termed here “hits” that have to be evaluated by experimental biochemical, structural-biological, molecular-biological or physiological methods for their expected biological activity. The term “molecular modeling” or “molecular modeling techniques” refers to techniques that generate one or more 3D models of a ligand binding site or other structural feature of a macromolecule. Molecular modeling techniques can be performed manually, with the aid of a computer, or with a combination of these. Molecular modeling techniques can be applied for example to the atomic co-ordinates to derive a range of 3D models and to investigate the structure of ligand binding sites. A variety of molecular modeling methods are available to the skilled person for use according to the invention (G. Klebe and H. Gohlke, Angew. Chem. Int. Ed. 2002, 41, 2644-2676; Jun Zeng: Combinatorial Chemistry & High Throughput Screening, 2000, 3, 355-362 355; Andrea G Cochran, Current Opinion in Chemical Biology 2001, 5:654-659).

In a preferred embodiment, the modulator is an inhibitor of coagulation factor XII activity, selected from the group consisting of: (a) an aptamer or inhibitory antibody or fragment or derivative thereof, specifically binding to a coagulation factor XII (poly)peptide and/or specifically inhibiting a coagulation factor XII activity; (b) a small molecule inhibitor of coagulation factor XII and/or coagulation factor XII activity; and (c) a serine protease inhibitor selected from group (I) consisting of wild-type and modified or engineered proteinaceous inhibitors of serine proteases including C1 esterase inhibitor, antithrombin III, α2-antiplasmin, α1-antitrypsin, ovalbumin serpins, and α2-macroglobulin, or selected from group (II) of Kunitz-type inhibitors including bovine pancreatic trypsin inhibitor.

The inhibitor can be an aptamer, preferably an aptamer specifically binding to coagulation factor XII. The term “aptamer” refers to RNA and also DNA molecules capable of binding target proteins with high affinity and specificity, comparable with the affinity and specificity of monoclonal antibodies. Methods for obtaining or identifying aptamers specific for a desired target are known in the art. Preferably, these methods may be based on the “systematic evolution of ligands by exponential enrichment” (SELEX) process (Ellington and Szostak, Nature, 1990, 346: 818-822; Tuerk and Gold, 1990, Science 249: 505-510; Fitzwater & Polisky, 1996, Methods Enzymol. 267: 275-301). Various chemical modifications, for example the use of 2′-fluoropyrimidines in the starting library and the attachment of a polyethylene glycol to the 5′ end of an aptamer can be used to ensure stability and to enhance bioavailability of aptamers (see e.g. Toulme 2000, Current Opinion in Molecular Therapeutics 2: 318-324).

The inhibitor can also be an antibody or fragment or derivative thereof. As used herein, the term “antibody or fragment or derivative thereof” relates to a polyclonal antibody, monoclonal antibody, chimeric antibody, single chain antibody, single chain Fv antibody, human antibody, humanized antibody or Fab fragment specifically binding to coagulation factor XII and/or to a mutant of coagulation factor XII.

The antibodies described herein may be prepared by any of a variety of methods known in the art. For example, polyclonal antibodies may be induced by administration of purified protein, a coagulation factor XII (poly)peptide or an antigenic fragment thereof, to a host animal.

As pointed out above, the antibody may also be a monoclonal antibody. Such monoclonal antibodies can be prepared using hybridoma technology (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., 1981, pp. 563-681). In general, such procedures involve immunizing an animal (preferably a mouse) with a coagulation factor XII protein antigen. The splenocytes of such immunized mice are extracted and fused with a suitable myeloma cell line. Any suitable myeloma cell line may be employed in accordance with the present invention; however, it is preferable to employ the parent myeloma cell line (SP2/0), available from the American Type Culture Collection, Rockville, Md. After fusion, the resulting hybridoma cells are selectively maintained in HAT medium, and then cloned by limiting dilution as described by Wands et al. 1981 (Gastroenterology 80:225-232). The hybridoma cells obtained through such a selection are then assayed to identify clones which secrete antibodies capable of binding the coagulation factor XII protein antigen.

It will be appreciated that Fab and F(ab′)₂ and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments).

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

Preferably, the antibodies specifically bind a coagulation factor XII (poly)peptide and include IgG (including IgG1, IgG2, IgG3, and IgG4), IgA (including IgA1 and IgA2), IgD, IgE, or IgM, and IgY. As used herein, the term “antibody” is meant to include whole antibodies, including single-chain whole antibodies, and antigen-binding fragments thereof. Most preferably the antibodies are human antigen binding antibody fragments and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are human, murine, rabbit, goat, guinea pig, camel, horse, or chicken.

“Specific binding” of antibodies may be described, for example, in terms of their cross-reactivity. Preferably, specific antibodies are antibodies that do not bind polypeptides with less than 98%, less than 95%, less than 90%, less than 85%, less than 80%, less than 75%, less than 70% and less than 65% identity (as calculated using methods known in the art) to a (poly)peptide encoded by the coagulation factor XII gene. Antibodies may, however, also be described or specified in terms of their binding affinity. Preferred binding affinities include those with a dissociation constant or Kd less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

Further, the inhibitor can be a “small molecule” or “small molecular compound”. As pointed out above, the term “small molecule” refers to a compound having a relative molecular weight of not more than 1000 D and preferably of not more than 500 D. Said compound may be of differing chemical nature, for example, it may be peptide-based or based on heterocyclic structures. Small molecule inhibitors of serine proteases have been extensively reviewed for example by Leung et al. 2000 (J. Med. Chem. 43: 305-341) and Walker & Lynas 2001 (Cell. Mol. Life. Sci. 58: 596-624). Substances discussed by these authors include, for example, (i) peptide-based inhibitors, like phosphorus-based inhibitors (including α-aminoalkyl diphenylphosphonate esters and mixed phosphonate esters), fluorine-containing inhibitors (including for example trifluoromethyl ketones [as well as analogues containing the trifluoromethyl ketone moiety with lower peptidic characteristics], difluoromethyl ketone-based and pentafluoroethyl ketone-based inhibitors), inhibitors based on peptidyl boronic acids (including, for example, boroArg- or boroLys- or boro-methoxy-propylglycine- or boroPro-containing substances), inhibitors based on so-called ‘inverse substrates’ (including, for example, compounds containing a p-methoxybenzoic acid function), and peptide-based inhibitors with novel functional groups (including, for example, compounds with C-terminal electron-withdrawing groups based on a-keto heterocycles, like a-keto benzoxazoles or a-keto thiazoles); (ii) natural product-derived inhibitors, like cyclotheonamides (macrocyclic pentapeptides analogues), aeruginosins, and radiosumin; (iii) inhibitors based on heterocyclic and other nonpeptide scaffolds, like N-hydroxysuccinimide heterocycles and related compounds, compounds based on the isocoumarin scaffold, and β-lactam-based inhibitors (including, for example, cephalosporin-derived compounds and analogues of monocyclic and bicyclic β-lactams); and (iv) metal-potentiated compounds, like compounds based on bis(5-amidino-2-benzimidazolyl)methane (BABIM). All these (types of) substances, as well as derivatives thereof, are considered to be applicable for the purposes of the present invention.

Any of the known protease inhibitors may be useful for developing modulators or inhibitory modulators of coagulation factor XII activity, although inhibitors of serine proteases may be particularly useful. Any of the known compounds may be modified, for example in order to change their binding characteristics or their specificity.

With respect to natural or engineered proteinaceous inhibitors of serine proteases, selective changes or modifications of the natural inhibitory characteristics, of the natural specificity have been achieved, for example, with P2 mutants of C1 inhibitor (Zahedi et al. 2001, J. Immunol. 167: 1500-1506), a P1 mutant of α1-antitrypsin (Schapira et al. 1985, J. Clin. Invest. 76: 645-647), various P1-P2-P3 mutants of α1-antitrypsin (Sulikowski et al. 2002, Protein Science 11: 2230-2236), a P1-P2 mutant of α1-antitrypsin (Schapira et al. 1987, J. Clin. Invest. 80: 582-585), various P3-P4 mutants of bovine pancreatic trypsin inhibitor (Grzesiak et al. 2000, J. Biol. Chem. 275: 33346-33352), among them one P3 mutant with high specificity for factor XIIa.

Particularly with respect to (a) and (b), it is also envisaged that the “inhibitor of coagulation factor XII activity” could be a compound that does not primarily target a coagulation factor XII (poly)peptide, but still inhibits coagulation factor XII activity, for example by inhibiting the activation of coagulation factor XII due to interference with an activating protein.

The present invention also relates to a method of identifying a compound modulating coagulation factor XII expression and/or secretion which is suitable as a medicament or lead compound for a medicament for the treatment and/or prevention of drug-associated angioedema, the method comprising the steps of: (a) in vitro contacting a cell that expresses or is capable of expressing coagulation factor XII with a potential modulator of expression and/or secretion; and (b) testing for altered expression and/or secretion, wherein the modulator is (i) a small molecule compound, an aptamer or an antibody or fragment or derivative thereof, specifically modulating expression and/or secretion of coagulation factor XII; or (ii) a siRNA or shRNA, a ribozyme, or an antisense nucleic acid molecule specifically hybridizing to a nucleic acid molecule encoding coagulation factor XII or regulating the expression of coagulation factor XII. “Specific hybridization” means that the siRNA, shRNA, ribozyme or antisense nucleic acid molecule hybridizes to the targeted nucleic acid molecule, encoding coagulation factor XII or regulating its expression. Preferably, “specific hybridization” also means that no other genes or transcripts are affected.

A modulating compound will affect expression and/or secretion of coagulation factor XII. The skilled person knows a number of techniques for monitoring an effect on protein expression or secretion. For example, protein expression may be monitored by using techniques such as western blotting, immunofluorescence or immunoprecipitation. Alternatively, expression may also, for example, be monitored by analyzing the amount of RNA transcribed from a coagulation factor XII gene.

The term “contacting a cell” refers to the introduction of a potential modulator compound into a cell. As far as the compound is a nucleic acid molecule, the contacting may be performed by any of the known transfection techniques such as electroporation, calcium phosphate transfection, lipofection and the like. However, the nucleic acid may also be entered into the cell by virus based vector systems.

As used herein, the term “siRNA” means “short interfering RNA”, the term “shRNA” refers to “short hairpin RNA”. In RNA interference, small interfering RNAs (siRNA) bind the targeted mRNA in a sequence-specific manner, facilitating its degradation and thus preventing translation of the encoded protein. Transfection of cells with siRNAs can be achieved, for example, by using lipophilic agents (among them Oligofectamine™ and Transit-TKO™) and also by electroporation.

Methods for the stable expression of small interfering RNA or short hairpin RNA in mammalian, also in human cells are known to the person skilled in the art and are described, for example, by Paul et al. 2002 (Nature Biotechnology 20: 505-508), Brummelkamp et al. 2002 (Science 296: 550-553), Sui et al. 2002 (Proc. Natl. Acad. Sci. U.S.A. 99: 5515-5520), Yu et al. 2002 (Proc. Natl. Acad. Sci. U.S.A. 99: 6047-6052), Lee et al. 2002 (Nature Biotechnology 20: 500-505), Xia et al. 2002 (Nature Biotechnology 20: 1006-1010). It has been shown by several studies that an RNAi approach is suitable for the development of a potential treatment of dominantly inherited diseases by designing a siRNA that specifically targets the disease-associated mutant allele, thereby selectively silencing expression from the mutant gene (Miller et al. 2003, Proc. Natl. Acad. Sci. U.S.A. 100: 7195-7200; Gonzalez-Alegre et al. 2003, Ann. Neurol. 53: 781-787).

The siRNA molecules are essentially double-stranded but may comprise 3′ or 5′ overhangs. They may also comprise sequences that are not identical or essentially identical with the target gene but these sequences must be located outside of the sequence of identity. The sequence of identity or substantial identity is at least 14 and more preferably at least 19 nucleotides long. It preferably does not exceed 23 nucleotides. Optionally, the siRNA comprises two regions of identity or substantial identity that are interspersed by a region of non-identity. The term “substantial identity” refers to a region that has one or two mismatches of the sense strand of the siRNA to the targeted mRNA or 10 to 15% over the total length of siRNA to the targeted mRNA mismatches within the region of identity. Said mismatches may be the result of a nucleotide substitution, addition, deletion or duplication etc. dsRNA longer than 23 but no longer than 40 bp may also contain three or four mismatches.

The interference of the siRNA with the targeted mRNA has the effect that transcription/translation is reduced by at least 50%, preferably at least 75%, more preferred at least 90%, still more preferred at least 95%, such as at least 98% and most preferred at least 99%.

Further, the modulator can be an antisense nucleic acid molecule specifically hybridizing to a nucleic acid molecule encoding coagulation factor XII or regulating the expression of coagulation factor XII. The term “antisense nucleic acid molecule” refers to a nucleic acid molecule which can be used for controlling gene expression. The underlying technique, antisense technology, can be used to control gene expression through antisense DNA or RNA or through triple-helix formation. Antisense techniques are discussed, for example, in Okano, J. Neurochem. 56: 560 (1991); “Oligodeoxynucleotides as Antisense Inhibitors of Gene Expression.” CRC Press, Boca Raton, Fla. (1988), or in: Phillips M I (ed.), Antisense Technology, Methods in Enzymology, Vol. 313, Academic Press, San Diego (2000). Triple helix formation is discussed in, for instance, Lee et al., Nucleic Acids Research 6: 3073 (1979); Cooney et al., Science 241: 456 (1988); and Dervan et al., Science 251: 1360 (1991). The methods are based on binding of a target polynucleotide to a complementary DNA or RNA. For example, the 5′ coding portion of a polynucleotide that encodes a coagulation factor XII (poly)peptide may be used to design an antisense RNA oligonucleotide of from about 10 to 40 base pairs in length. A DNA oligonucleotide is designed to be complementary to a gene region involved in transcription thereby preventing transcription and the production of coagulation factor XII. The antisense RNA oligonucleotide hybridizes to the mRNA in vivo and blocks translation of the mRNA molecule into coagulation factor XII polypeptide.

The term “ribozyme” refers to RNA molecules with catalytic activity (see, e.g., Sarver et al, Science 247:1222-1225 (1990)); however, DNA catalysts (deoxyribozymes) are also known. Ribozymes and their potential for the development of new therapeutic tools are discussed, for example, by Steele et al. 2003 (Am. J. Pharmacogenomics 3: 131-144) and by Puerta-Fernandez et al. 2003 (FEMS Microbiology Reviews 27: 75-97). While ribozymes that cleave mRNA at site specific recognition sequences can be used to destroy coagulation factor XII mRNAs, the use of trans-acting hairpin or hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). There are numerous potential hammerhead ribozyme cleavage sites within the nucleotide sequence of the coagulation factor XII mRNA which will be apparent to the person skilled in the art. Preferably, the ribozyme is engineered so that the cleavage recognition site is located near the 5′ end of the coagulation factor XII mRNA; i.e., to increase efficiency and minimize the intracellular accumulation of non-functional mRNA transcripts. RNase P is another ribozyme approach used for the selective inhibition of pathogenic RNAs. Ribozymes may be composed of modified oligonucleotides (e.g. for improved stability, targeting, etc.) and should be delivered to cells which express coagulation factor XII. DNA constructs encoding the ribozyme may be introduced into the cell in the same manner as described above for the introduction of other nucleic acid molecules. A preferred method of delivery involves using a DNA construct “encoding” the ribozyme under the control of a strong constitutive promoter, such as, for example, pol III or pol II promoter, so that transfected cells will produce sufficient quantities of the ribozyme to destroy endogenous coagulation factor XII messages and inhibit translation. Since ribozymes unlike antisense molecules, are catalytic, a lower intracellular concentration is generally required for efficiency. Ribozyme-mediated RNA repair is another therapeutic option applying ribozyme technologies (Watanabe & Sullenger 2000, Adv. Drug Deliv. Rev. 44: 109-118) and may also be useful for the purpose of the present invention. To this end, catalytic group I introns can be employed in a trans-splicing reaction to replace a defective segment of target mRNA.

In a preferred embodiment of the method of the present invention, coagulation factor XII is a disease-associated mutant of coagulation factor XII. As pointed out above, in order to determine whether or not a mutation is disease-associated, the person skilled in the art may, for example, compare the frequency of a specific sequence change, for example in the coagulation factor XII gene, in patients affected by the disease, having developed an angioedema in association with the administration of a particular drug, with the frequency in appropriately chosen control individuals and conclude from a statistically significantly deviating frequency in the patient group that said mutation is a disease-associated mutation.

In another preferred embodiment of the present invention, said modulator is selective for a disease-associated mutant of coagulation factor XII, the method comprising (a) comparing the effect of the modulator on wild-type and disease-associated coagulation factor XII activity or their expression and/or secretion; and (b) selecting a compound which (i) modulates disease-associated coagulation factor XII activity or its expression and/or secretion and which (ii) does not affect wild-type coagulation factor XII activity or its expression and/or secretion. By using this method, the skilled person can determine whether a modulating compound is a general modulator of coagulation factor XII or selective for disease-associated coagulation factor XII. It is also possible and envisaged that a modulator affects preferably disease-associated coagulation factor XII, and partially, but to a lesser extent, also wild-type coagulation factor XII.

In yet another preferred embodiment of the present invention's methods, the disease-associated mutant or mutation is: (a) a mutant located in the fibronectin type II domain, within the region of amino acid position 1 to 76, and/or a mutation located in the nucleic acid sequence encoding the fibronectin type II domain, within mRNA position 107 to 334; (b) a mutant located in the EGF-like domain 1, within the region of amino acid position 77 to 113, and/or a mutation located in the nucleic acid sequence encoding the EGF-like domain 1, within mRNA position 335 to 445; (c) a mutant located in the fibronectin type I domain, within the region of amino acid position 114 to 157, and/or a mutation located in the nucleic acid sequence encoding the fibronectin type I domain, within mRNA position 446 to 577; (d) a mutant located in the EGF-like domain 2, within the region of amino acid position 158 to 192, and/or a mutation located in the nucleic acid sequence encoding the EGF-like domain 2, within mRNA position 578 to 682; (e) a mutant located in the kringle domain, within the region of amino acid position 193 to 276, and/or a mutation located in the nucleic acid sequence encoding the kringle domain, within mRNA position 683 to 934; (f) a mutant located in the proline-rich region, within the region of amino acid position 277 to 331, and/or a mutation located in the nucleic acid sequence encoding the proline-rich region, within mRNA position 935 to 1099; (g) a mutant located in the region of proteolytic cleavage sites, within the region of amino acid position 332 to 353, and/or a mutation located in the nucleic acid sequence encoding the region of proteolytic cleavage sites, within mRNA position 1100 to 1165; (h) a mutant located in the serine protease domain, within the region of amino acid position 354 to 596, and/or a mutation located in the nucleic acid sequence encoding the serine protease domain, within mRNA position 1166 to 1894; (i) a mutant located in the signal peptide, within the region of amino acid position −19 to −1, and/or a mutation located in the nucleic acid sequence encoding the signal peptide, within mRNA position 50 to 106; (j) a mutation located in the untranslated regions (UTRs) of coagulation factor XII mRNA, within mRNA position 1 to 49 and/or 1895 to 2048; (k) a mutation located in an intron of the coagulation factor XII gene; and/or (l) a mutation located in a flanking regulatory genomic sequence of the coagulation factor XII gene, within the region encompassing 4000 bp upstream of the transcription initiation site of the coagulation factor XII gene and/or within the region encompassing 3000 bp downstream of the nucleotide sequence representing the 3′-UTR of the coagulation factor XII mRNA.

The above numbering of amino acid residues of human coagulation factor XII refers to the numbering as given for example in Cool & MacGillivray 1987 (J. Biol. Chem. 262: 13662-13673). The numbering of mRNA positions refers to GeneBank acc. no. NM 000505.2. Introns of the coagulation factor XII gene are preferably introns one to thirteen as given for example in the Seattle data (http://pga.gs.washington.edu/data/f12/f12.ColorFasta.html) or in the UCSC Genome Browser/July 2003 human reference sequence/chr5:176,810,093-176,817,530. Also according to the July 2003 human reference sequence of the UCSC Genome Browser, flanking regulatory sequences of the coagulation factor XII gene, as given above, encompass nucleotide positions chr5:176,817,531 to 176,821,030 and nucleotide positions chr5:176,807,093 to 176,810,092.

Recently, newly identified mutations of the coagulation factor XII gene, namely two mutations in exon 9 encoding the proline-rich region of factor XII (g.6927C>A; g.6927C>G; numbering according to GenBank acc. No. AF 538691), as well as a mutation in intron 2 of the coagulation factor XII gene (g.4278C>T; numbering according to GenBank acc. No. AF 538691) have been found to be significantly associated with a novel type of familial/hereditary angioedema (hereditary angioedema with normal C1 inhibitor, hereditary angioedema type III). It is envisaged that these mutations are also associated with the diseases of the present invention, with the development of a drug-associated angioedema or drug-associated angioedema-related symptoms. It is reasonable to assume that individuals carrying a mutation associated with the above type of familial angioedema are also prone to develop a drug-associated angioedema or drug-associated angioedema-related symptoms.

These mutations may thus be useful in accordance with the teaching of the present invention. In particular, the methods disclosed herein may e.g. be carried out by testing for the presence and/or absence of said mutations and or mutants.

Accordingly, it is envisaged that said disease-associated mutant located in the proline-rich region is a mutant affecting the threonine residues 309 or 310 of mature coagulation factor XII, more preferably a mutant affecting the Thr309 residue, even more preferably a mutant substituting the Thr309 residue by a lysine or arginine residue, and/or that said disease-associated mutation located in the nucleic acid sequence encoding the proline-rich region is a mutation within genomic DNA positions 6926 to 6931 (numbering according to GenBank acc. No. AF 538691), more preferably a mutation at position g.6927 and even more preferably a mutation substituting the wild-type C to either an A or a G. Moreover, accordingly, it is also envisaged that said disease-associated mutation located in an intron of the coagulation factor XII gene is a mutation located in intron 2 of the coagulation factor XII gene, preferably (but not exclusively) a mutation in position 4278 of the genomic sequence given under GenBank acc. No. AF 538691, even more preferably a substitution of the wild-type C by a T (g.4278C>T).

In a preferred embodiment, the present invention's method comprises the additional step of producing the modulator identified in said methods.

In another preferred embodiment, the present invention's method comprises in vitro testing of a sample of a blood donor for determining whether the blood of said donor or components thereof may be used for transfusion to a patient in need thereof, wherein a positive testing indicates a predisposition for drug-associated angioedema, excluding the transfusion of blood or components thereof from said donor. A patient in need of a transfusion might be multimorbide and might well be receiving a medication comprising a drug considered for the present invention.

Particularly in such a case the transfusion with blood or blood products from an individual with a predisposition to develop drug-associated angioedema might include a health hazard for the recipient of the blood in that the recipient could become affected by angioedema attacks, albeit only for a transient period.

In another preferred embodiment of the present invention, said drug is selected from the group consisting of (a) angiotensin-converting enzyme (ACE) inhibitors; (b) angiotensin II receptor type 1 (AT₁) antagonists (sartans); (c) fibrinolytic or thrombolytic drugs; (d) vasopeptidase inhibitors; (e) neutral endopeptidase (NEP) inhibitors; (f) inhibitors of endothelin-converting enzyme 1 (ECE-1); (g) triple inhibitors of ECE-1, NEP and ACE; (h) inhibitors of dipeptidyl peptidase IV (DPP IV); (i) calcium channel blockers; (j) estrogens and estrogen-like drugs; (k) anti-androgens; and (l) corticosteroids.

For all these drugs or classes of drugs the development of angioedema as an adverse drug reaction it is either known or may be envisaged.

Some details on these various drugs or drug classes, considered to be of relevance for the purpose of the present invention, are given in the following paragraphs.

(a) Angiotensin-converting enzyme (ACE) inhibitors (and medications containing ACE inhibitors in combination with other substances): Angioedema is known as a potentially life-threatening adverse effect of ACE inhibitor therapy and has been associated with all ACE inhibitors commercially available, irrespective of chemical structure (Vleeming et al. 1998, Drug Safety 18: 171-188). It is estimated to have an incidence of 0.1 to 0.5% among Caucasians, whereas a considerably higher incidence has been observed in black patients (Brown et al. 1996, Clin. Pharmacol. Ther. 60: 8-13; Gibbs et al. 1999, Br. J. Clin. Pharmacol. 48: 861-865).

It is generally assumed that bradykinin is probably an important mediator of the angioedema associated with the use of ACE inhibitors. ACE, also known as kininase II, mediates the conversion of angiotensin I to angiotensin II as well as the degradation of bradykinin. As a result of the inhibition of ACE, circulating and tissue levels of angiotensin II are decreased, whereas bradykinin levels are increased. Pellacani et al. 1994 (Clin. Sci. 87: 567-574) demonstrated that pharmacological inhibition of ACE leads to increased plasma levels of bradykinin, and high levels of bradykinin have been demonstrated in plasma during acute angioedema episodes (Nussberger et al. 1998, Lancet 351: 1693-1697; Cugno et al. 2003, Int. Immunopharmacol. 3: 311-317).

Considering the widespread use of ACE inhibitors (it is estimated that, at present, 35 to 40 million patients worldwide are treated with these drugs) it is not surprising that even conservative calculations might suggest that this drug class could account for several hundred deaths per year due to fatal angioedema attacks affecting the larynx or upper respiratory tract (Messerli & Nussberger 2000, Lancet 356: 608-609).

Marketed substances are for example captopril, enalapril, lisinopril, quinapril, perindopril, ramipril, cilazapril, fosinopril, trandolapril, moexipril, benazepril, spirapril, imidapril. Many of these substances are also used in fixed combinations (e.g. with hydrchlorothiazide). Other ACE inhibitors discussed in the literature and also envisaged to be of importance for the present invention include e.g. abutapril, alacepril, ceronapril, delapril, idrapril, pentopril, rentiapril, temocapril, zabicipril, and zofenapril (Barr 1994, Teratology 50: 399-409). There are still numerous ongoing clinical trials involving various ACE inhibitors (Nadar & Lip 2002, Expert Opin. Investig. Drugs 11: 1633-1643).

(b) Angiotensin II receptor type 1 (AT₁) antagonists (sartans) (and medications containing sartans in combination with other substances): At present, seven different angiotensin II receptor type 1 antagonists are marketed, namely losartan, valsartan, irbesartan, candesartan, telmisartan, eprosartan, and olmesartan. Further substances are in clinical development, e.g. ripisartan, YM-358, GA-0056, CL-329167 (Burnier 2001, Circulation 103: 904-912; Wexler et al. 1996, J. Med. Chem. 39: 625-656). There are published reports on angioedema in association with the use of telmisartan (Borazan et al. 2003, Allergy 58: 454; Howes & Tran, 2002, Drug Safety 25: 73-76), valsartan (Frye & Pettigrew 1998, Pharmacotherapy 18: 866-868; de la Serna Higuera 2000, Med. Clin. (Barc) 114: 599; Martinez Alonso et al. 2003, Allergy 58: 367-369; Irons & Kumar 2003, Ann. Pharmacother. 37: 1024-1027), losartan (Acker & Greenberg 1995, N. Engl. J. Med. 333: 1572; Boxer 1996, J. Allergy Clin. Immunol. 98: 471; van Rijnsoever et al. 1998, Arch. Intern. Med. 158: 2063-2065; Cha & Pearson 1999, Ann. Pharmacother. 33: 936-938; Rivera 1999, Ann. Pharmacother. 33: 933-935; Rupprecht et al. 1999, Allergy 54: 81-82; Sharma & Yium 1997, South. Med. J. 90: 552-553; Chiu et al. 2001, Laryngoscope 111: 1729-1731; Abdi et al. 2002, Pharmacotherapy 22: 1173-1175; de Paz et al. 1999, Med. Clin. (Barc) 113: 759; Howes & Tran, 2002, Drug Safety 25: 73-76), irbesartan (Rodriguez Conesa et al. 2001, Rev. Esp. Cardiol. 54: 532; Howes & Tran, 2002, Drug Safety 25: 73-76), eprosartan (Howes & Tran, 2002, Drug Safety 25: 73-76) and candesartan (Lo 2002, Pharmacotherapy 22: 1176-1179; Hille et al. 2003, Am. J. Ophthalmol. 135: 224-226). Angioedema is also listed as a side-effect of olmesartan.

It is generally assumed that angioedema occurs less frequently with angiotensin II receptor type 1 antagonists than with ACE inhibitors.

The pathogenetic mechanism of angioedema attributable to angiotensin II receptor antagonists is not clear. Angiotensin II receptor antagonists block the vasoconstrictor and aldosteron-secreting effects of angiotensin II by selectively blocking the angiotensin II type 1 (AT₁) receptor and are primarily not expected to increase bradykinin plasma concentration, unlike ACE inhibitors.

However, recently published animal data increasingly support the concept of a link between AT₁ receptor blockade and an increase in local tissue bradykinin levels, possibly mediated via an activation of the unopposed AT₂ receptor by increased concentration of angiotensin II after displacement from AT₁ receptor. For example, Zhu et al. 1999 (J. Cardiovasc. Pharmacol. 33: 785-790) demonstrated that treatment with losartan protected rat hearts against acute ischemia-reperfusion injury; these effects apparently were dependent on bradykinin, because treatment with the bradykinin B2 receptor antagonist Hoe140 abolished the beneficial effects of pre- and post-ischemically administered losartan. Also in rats, valsartan treatment caused a significant increase of bradykinin levels in renal interstitial fluid (Siragy et al. 2001; Hypertension 38: 183-186); the effects of valsartan on bradykinin levels were abolished by specific AT₂ receptor blockade with PD 123,319, supporting the assumption that angiotensin II stimulation of the AT₂ receptor is involved in these effects. A negative-feedback effect of increased concentrations of angiotensin II on ACE (Schunkert et al. 1993, Circ. Res. 72: 312-318) might also play a role with respect to the potential effects of sartans on local bradykinin levels. Under certain conditions, AT₁ receptor blockade might, finally, increase local bradykinin concentrations via decreasing the activity of neutral endopeptidase (Walther et al. 2002; FASEB J. 16: 1237-1241).

(c) Fibrinolytic or thrombolytic drugs (recombinant tissue plasminogen activator (rtPA; alteplase), urokinase, streptokinase, and related drugs or medications): Angioedema has also been observed after therapy with various fibrinolytic drugs [recombinant tissue plasminogen activator (rtPA, alteplase), urokinase, and streptokinase] in patients with acute myocardial infarction, acute ischemic stroke, and deep vein thrombosis (Agostoni & Cicardi 2001, Drug Safety 24: 599-606; Walls & Pollack 2000, Ann. Emerg. Med. 35: 188-191; Pechlaner et al. 2001, Blood Coag. Fibrinolysis 12: 491-494). In a prospective study on 176 patients treated with IV alteplase for acute ischemic stroke 5.1% of the patients developed orolingual angioedema (Hill et al. 2003, Neurology 60: 1525-1527).

Strongly supporting assumptions on a role of bradykinin, Molinaro et al. 2002 (Stroke 33: 1712-1716) demonstrated the kinin-forming activity of rtPA in plasma. It depends on its activation of plasminogen into plasmin, which in turn activates the different constituents of the contact system of plasma. The demonstration of an enhanced kinin release from high molecular weight kininogen by plasma kallikrein after its exposure to plasmin (Kleniewski et al. 1992, J. Lab. Clin. Med. 120: 129-139) might well support the importance of in vivo plasminogen activation for a pathophysiologically effective kinin production.

Considering these findings it is envisaged, in accordance with the invention, that also numerous newer drugs being developed for thrombolytic therapies (as those reviewed e.g. by Lapchak 2002, Expert Opin. Investig. Drugs 11: 1623-1632; for example tenecteplase, lanoteplase, reteplase, monteplase, duteplase, nateplase, pamiteplase, and others like HTU-PA, pro-urokinase A-74187, or bat-tPA/rDSPA alpha-1) may be associated with an increased risk of angioedema development.

(d) Vasopeptidase inhibitors (and medications containing vasopeptidase inhibitors in combination with other substances): Vasopeptidase inhibitors represent a new class of cardiovascular drugs presently under development (Campbell 2003, Hypertension 41: 383-389; Nawarskas et al. 2001, Heart Disease 3: 378-385). They possess the ability to simultaneously inhibit angiotensin-converting enzyme (ACE) and neutral endopeptidase (NEP); thus, they decrease angiotensin II generation by inhibiting ACE activity and reduce the metabolic degradation of natriuretic peptides by inhibiting NEP.

Several substances have been investigated preclinically and/or clinically, namely e.g. omapatrilat, fasidotril, sampatrilat, mixanpril (S 21402, active metabolite RB 105), MDL 100,240 (active metabolite MDL 100,173), Z13752A, BMS189921, and AVE 7688 (Nawarskas et al. 2001, Heart Disease 3: 378-385; Campbell 2003, Hypertension 41: 383-389; Nathisuwan & Talbert 2002, Pharmacotherapy 22: 27-42; Weckler et al. 2003, J. Renin Angiotensin Aldosterone Syst. 4: 191-196). The most clinically advanced vasopeptidase inhibitor is omapatrilat (Vanlev, BMS 186716) (Zanchi et al. 2003, Current Hypertension Reports 5: 346-352), and several large trials have addressed its use in patients with hypertension and patients with congestive heart failure, the two main indications for which vasopeptidase inhibitors are targeted.

Both enzymes inhibited by vasopeptidase inhibitors, ACE as well as NEP, also inactivate bradykinin, and ACE inhibitors alone have been shown to increase plasma kinin concentrations (Pellacani et al. 1994). Thus, it might not be surprising that angioedema appears to be more common in patients receiving a vasopeptidase inhibitor (omapatrilat) than in patients receiving an ACE inhibitor (Zanchi et al. 2003; Campbell 2003; Messerli & Nussberger 2000, Lancet 356: 608-609). As with ACE inhibition alone, the rate of angioedema appears to be especially high in black patients (Zanchi et al. 2003).

Concerns regarding reports of angioedema in patients treated with omapatrilat have delayed the regulatory approval of this compound for human use (Messerli & Nussberger 2000; Nawarskas et al. 2001). The further clinical development of vasopeptidase inhibitors in general, and the advanced compound omapatrilat in particular, might well benefit from the potential identification of individuals being at high risk for developing angioedema in order to exclude such individuals from the prescription of these compounds.

Vasopeptidase inhibitors may be combined with other substances, e.g. hydrochlorothiazide (Nathisuwan & Talbert 2002).

(e) Neutral endopeptidase (NEP) inhibitors (and medications containing NEP inhibitors in combination with other substances): Neutral endopeptidase (NEP) is one of the enzymes involved in the inactivation/degradation of bradykinin, and it is assumed that NEP inhibition can potentiate the accumulation of bradykinin, can increase local bradykinin concentrations (Duncan A. M. et al. 1999, J. Pharmacol. Exp. Ther. 289: 295-303; Dumoulin M.-J. et al. 2001, J. Cardiovasc. Pharmacol. 37: 359-366). Thus, it is envisaged, in accordance with the invention, that the administration of NEP inhibitors may increase the risk for the development of angioedema.

Preclinical and clinical studies have investigated the combined use of NEP inhibitors and ACE inhibitors (Campbell 2003), demonstrating e.g. that blood pressure was reduced more effectively in hypertensive patients than with ACE or NEP inhibition alone. It is envisaged that such a combined use may be associated with a further increased risk of angioedema, eventually comparable to the risk seen with vasopeptidase inhibition.

NEP inhibitors studied are for example thiol inhibitors (e.g. thiorphan and retrothiorphan, acetorphan, SQ29072), carboxyl inhibitors (e.g. SCH39370, UK69578 [candoxatrilat]), phosphoryl and hydroxamate inhibitors (e.g. phosphoramidon, RS kelatorphan, RB1047.8); further substances are e.g. BP102 and ecadotril (Nawarskas et al. 2001, Heart Disease 3: 378-385).

(f) Inhibitors of endothelin-converting enzyme 1 (ECE-1) (and related medications, including combined ECE-1/NEP inhibitors): Endothelin-converting enzyme 1 (ECE-1) is the key enzyme in the production of the potent vasoconstrictor endothelin from its inactive precursor big endothelin. However, it is also involved in the hydrolysis of bradykinin (Hoang M. V. & Turner A. J. 1997; Biochem J. 327: 23-26). Some inhibitors of neutral endopeptidase apparently inhibit also ECE-1 (e.g. phosphoramidon, SLV-306 (KC-12792, KC 12615), S-17162, CGS 26303, CGS 26393, CGS 31447, WS 75624B, RU 69738, B90063, CGS 34043, and others) (Hoang & Turner 1997; Tabrizchi R. 2003, Curr. Opin. Investig. Drugs 4: 329-332; Battistini & Jeng 2001, Handbook of Exp. Pharmacol. 152: 155-208).

Thus, without being bound by any theory, it is believed, in accordance with the invention, that ECE-1 inhibitors and combined ECE-1/NEP inhibitors may increase local bradykinin concentrations and—by that—the risk for the development of angioedema.

ECE-1 inhibitors have been shown to be efficacious in experimental models of hypertension, chronic heart failure, cerebral vasospasm following subarachnoid hemorrhage, and renal failure.

Various classes of ECE-1 inhibitors (including peptides, natural products, and low molecular weight molecules featuring a phosphorus-containing functionality, a sulfhydryl, a hydroxamic acid, or a carboxylic acid as the zinc-binding group), also their design and pharmacological properties, have been extensively reviewed e.g. by Jeng & DeLombaert 1997 (Current Pharmacological Design 3: 597-614), DeLombaert et al. 2000 (J. Med. Chem. 43: 488-504), Battistini & Jeng 2001 (Handbook of Exp. Pharmacol. 152: 155-208).

ECE-1 inhibitors are e.g.: CGS30084 (and analogues), CGS26303, CGS34225 (orally active prodrug of CGS 34226) (Trapani et al. 2002, Clinical Science 103 (Suppl. 48): 102S-106S), R00687629, CGS 34226 (combined ECE-1/NEP inhibitor) (Jeng et al. 2002, Clinical Science 103 (Suppl. 48): 98S-101S), CGS35066 (Battistini et al. 2002, Clinical Science 103 (Suppl. 48): 363S-366S), FR901533 (Wada et al. 2002, Clinical Science 103 (Suppl. 48): 254S-257S), CGS26303 (prodrug is CGS26393; Kwan et al. 2002; Clinical Science 103 (Suppl. 48): 414S-417S) (combined ECE-1/NEP inhibitor).

(g) Triple inhibitors of ECE (ECE-1), NEP, and ACE (and medications containing such triple inhibitors in combination with other substances): All three enzymes being zinc-metalloproteases, the potential design of such triple inhibitors for example as antihypertensive agents has been considered (Jeng et al. 2002). In fact, a number of substances are under study (Battistini & Jeng 2001, Handbook of Exp. Pharmacol. 152: 155-208); among these are phosphinic acid derivatives (for example SCH 54470), phosphonamides, a non-peptidic aminophosphonic acid, and other compounds like SA6817 or CGS26582. Naturally, from the aforementioned data (regarding inhibition of bradykinin degradation mediated by these enzymes), it may be expected that such inhibitors will be associated with a considerably increased risk for angioedema.

(h) Inhibitors of dipeptidyl peptidase IV (DPP IV) (and medications containing DPP IV inhibitors in combination with other substances): Dipeptidyl peptidase IV (DPP IV) is another peptidase that is potentially involved in the degradation of bradykinin (Vanhoof et al. 1992, Agents Actions Suppl. 1992, 38: 120-127; Lefebvre et al. 2002, Hypertension 39: 460-464; Pesquero et al. 1992, J. Hypertens. 10: 1471-1478); it is also involved in the degradation of substance P (Wang et al. 1991, Peptides 12: 1357-1364). Thus, it is envisaged, in accordance with the invention, that pharmacological inhibition of DPP IV may be associated with an increased risk for the development of angioedema.

Several inhibitors of DPP IV are progressing through preclinical and clinical trials, for example for the treatment of type 2 diabetes mellitus (Drucker 2003, Expert Opin. Investig. Drugs 12: 87-100; Hoist 2003, Adv. Exp. Med. Biol. 524: 263-279; Rosenblum & Kozarich 2003, Curr. Opinion Chem. Biol. 7: 496-504). Substances are e.g. vildagliptin, sitagliptin, saxagliptin, alogliptin. Without being bound by any theory, it is believed, in accordance with the invention, that such substances may increase the risk for angioedema development, in particular if administered to individuals, in whom a genetically determined susceptibility favours an enhanced activation of the kinin pathway, an increased kinin formation.

(i) Calcium channel blockers (amlodipine and related substances, and medications containing these calcium channel blockers in combination with other substances): Zhang X. et al. 1999 (Am. J. Cardiol. 84: 27L-33L) demonstrated that the increase in NO production induced by amlodipine was apparently kinin-mediated: HOE-140, a bradykinin receptor antagonist, entirely abolished the increase in NO production induced by amlodipine.

For at least two further Ca channel blockers, namely nifedipine and benidipine, it is also assumed that increases in NO levels are due to bradykinin-dependent mechanisms (Kitakaze et al. 2000, Circulation 101: 311-317; Asanuma et al. 2001, Cardiovasc. Drugs Ther. 15: 225-231). Benidipine has been reported to activate kallikrein (Yoshida et al. 1996, J. Hypertension 14: 215-222).

Angioedema is listed as a side-effect of nifedipine as well as amlodipine. Angioedema is also listed as a side-effect for nisoldipine (another marketed Ca channel blocker of the dihydropyridine type). Finally, angioedema has also been described as a side-effect of nicardipine (Sauve et al. 1999, Therapie 54: 63-65). It is envisaged that in certain individuals, in whom a genetically determined susceptibility favours an enhanced activation of the kinin pathway or an increased kinin generation, a medication-induced kinin increase may superimpose with that genetic condition and thus may increase the risk for developing angioedema.

(j) Estrogens and estrogen-like drugs (and medications containing estrogens or estrogen-like drugs in combination with other substances): Numerous reports have described an influence of estrogens and/or estrogen-containing medications on various components of the kinin system, thus also affecting the synthesis and degradation of bradykinin. Plasma C1 inhibitor levels are reduced in women taking oral contraceptives (Gordon et al. 1980, J. Lab. Clin. Med. 96: 762-769), the administration of estrogens (estrogen-containing medications) also leads to increased levels of factor XII (Gordon et al. 1980, J. Lab. Clin. Med. 96: 762-769; Gevers Leuven et al. 1987, J. Lab. Clin. Med. 109: 631-636; Gordon et al. 1988, J. Lab. Clin. Med. 111: 52-56; Citarella et al. 1996, Steroids 61: 270-276). Plasma prekallikrein levels are increased in women using estrogen-containing oral contraceptives (Fossum et al. 1994, Thromb. Res. 74: 477-485). Estrogen-containing medications also influence the degradation of bradykinin by decreasing ACE activity (Proudler et al. 1995, Lancet 346: 89-90; Schunkert et al. 1997, Circulation 95: 39-45; Nogawa et al. 2001, Menopause 8: 210-215). Studies in ovariectomized rats receiving 17β-estradiol replacement therapy or placebo suggest that estrogen treatment regulates tissue ACE activity by reducing ACE mRNA concentrations (Gallagher et al. 1999, Hypertension 33: 323-328).

All these effects are in favor of an increased kinin production or a kinin accumulation due to a decreased kinin degradation. Thus, it is not surprising that angioedema has been described in association with the use of various estrogens (estrogen-containing medications) (Andre et al. 2003, Toxicology 185: 155-160; Bouillet et al. 2003, Dermatology 206: 106-109; Nettis et al. 2001, Immunopharmacol. Immunotoxicol. 23: 585-595; Bork et al. 2003, Am. J. Med. 114: 294-298), often but not exclusively in patients with some type of underlying hereditary angioedema.

Relevant substances can be, for example, estradiol, estradiolvalerat, estradiolbenzoat, ethinylestradiol, conjugated estrogens (conjugated equine estrogens), estriol, mestranol.

Medications most commonly involved are various oral contraceptives (in particular, but not exclusively combinations with ethinylestradiol) (but also other (non-oral) pharmaceutical compositions for contraception) and pharmaceutical compositions used for hormone replacement therapy.

However, estrogens and/or estrogen-like substances are also used as antineoplastic therapeutics. Substances to be mentioned here are, for example, polyestradiol (-phosphat), fosfestrol, and ethinylestradiolpropansulfonat. It is envisaged that such substances, due to potential estrogen-like effects on components of the kinin system, may well increase the risk for development of angioedema attacks.

Another substance, envisaged to be of importance with respect to the present invention, is estramustine phosphate (EMP), a nornitrogen mustard carbamate derivative of estradiol-17β phosphate displaying both estrogenic and cytotoxic activities; angioedema is listed as a potential side-effect of this substance. Hernes et al. 1997 (Br. J. Cancer 76: 93-99) reported that two of 24 patients receiving EMP within a combination therapy (with epirubicin) experienced recurrent angioedema.

(k) Anti-androgens (and medications containing anti-androgens in combination with other substances): A potential association between recurrent angioedema and an anti-androgen therapy (or an androgen-deficit) has been suggested by Pichler et al. 1989 (Ann. Allergy 63: 301-305). These authors described recurrent angioedema in four women using cyproteronacetate-containing contraceptives, as well as in two men with hypogonadal hypogonadism. The cessation of angioedema after stopping the cyproteronacetate-containing contraceptive in spite of the further intake of gestagen/estrogenic compounds strongly suggests that the cyproteronacetate content of the contraceptive increased the angioedema susceptibility.

It is envisaged that, beside cyproteron acetate, relevant antiandrogens, according to the invention, may include, for example, chlormadinonacetate and eventually also antineoplastic antiandrogens like flutamid or bicalutamid.

(l) Corticosteroids (methylprednisolone and related drugs) (and medications containing methylprednisolone and/or related drugs in combination with other substances): Roeise et al. 1990 (Thromb. Res. 57: 877-888) demonstrated in an in vitro study that methylprednisolone in a dose-dependent way activates the contact system (the kinin-forming pathway) of plasma, as measured e.g. as rapid and marked increases of kallikrein activity. Activation was even seen in plasma containing relatively low doses of methylprednisolone, doses which are equivalent to doses used in clinical practice. Methylprednisolone also facilitated the endotoxin-induced activation of the plasma kallikrein-kinin system (Roeise & Aasen 1989, Surg. Res. Comm. 5: 41-47). The occurrence of sudden deaths after rapid injections of methylprednisolone as occasionally reported might also relate to such effects on the kinin-forming pathway (Roeise et al. 1990; Bocanegra et al. 1981, Ann. Intern. Med. 95: 122).

Without being bound by any theory, it is believed, in accordance with the invention, that in certain individuals, in whom a genetically determined susceptibility favours an enhanced activation of the kinin pathway or facilitates an increased kinin generation, an activation of the contact system due to a corticosteroid medication like methylprednisolone may superimpose with that genetic condition and thus may increase the possibility of angioedema development.

With respect to all the aforementioned drugs, the present invention also relates to medications that contain these drugs in combination with one or more other substances, like, for example, in the case of an antihypertensive drug, a diuretic compound. In addition, with respect to all these drug classes, related drugs, i.e. drugs with the same biochemical effect or pharmacological mechanism of action (like inhibition of a specific enzyme), are to be considered for the present invention, too.

Synergism between various of these drug classes in the generation of angioedema in case of simultaneous administration to a patient also has to be considered.

The present invention also relates to the use of (a) a (poly)peptide encoded by the coagulation factor XII gene or a fragment thereof, (b) a modulator of coagulation factor XII identified by any of the methods of the present invention; (c) a nucleic acid molecule capable of expressing coagulation factor XII or a fragment thereof; and/or (d) a nucleic acid molecule capable of expressing a modulator of coagulation factor XII activity or its expression and/or secretion, for the preparation of a pharmaceutical composition for the treatment and/or prevention of drug-associated angioedema. Said modulator of coagulation factor XII may be any of the modulating compounds identified by the methods of the present invention or any of the modulating compounds disclosed in the present invention. As such, the modulator may be affecting the expression from the coagulation factor XII gene or may modulate the secretion or function of coagulation factor XII. Preferably, the modulating compound is an inhibitor of coagulation factor XII activity or of its expression or secretion. The use of (a) and (c) may be envisaged, for example, with the purpose of a vaccination, either protein-based or DNA-based, to stimulate an immune response against coagulation factor XII (vide infra).

The active components of a pharmaceutical composition such as, e.g. a small molecular compound or an antibody, will be formulated and dosed in a fashion consistent with good medical practice, taking into account the clinical condition of the individual patient, the site of delivery of pharmaceutical composition, the method of administration, the scheduling of administration, and other factors known to practitioners. The “effective amount” of the components of the pharmaceutical composition for purposes herein is thus determined by such considerations.

As a general proposition, the total pharmaceutically effective amount of for example a proteinaceous compound administered parenterally per dose will be in the range of about 1 μg/kg/day to 10 mg/kg/day of patient body weight, although, as noted above, this will be subject to therapeutic discretion. The length of treatment needed to observe changes and the interval following treatment for responses to occur appears to vary depending on the desired effect. Pharmaceutical compositions may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers for example to modes of administration which include intravenous, intramuscular, intraperitoneal, intrasternal, subcutaneous and intraarticular injection and infusion.

The pharmaceutical composition is also suitably administered by sustained-release systems. Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release compositions also include for example liposomally entrapped components. Liposomes containing the active components of the pharmaceutical composition are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

Components to be used for therapeutic administration must be sterile. Sterility is readily accomplished by filtration through sterile filtration membranes (e.g., 0.2 micron membranes). Therapeutic compositions generally are placed into a container having a sterile access port, for example, an intravenous solution bag or vial having a stopper pierceable by a hypodermic injection needle.

It is further envisaged in a preferred embodiment of the present invention's use, that said coagulation factor XII or said (poly)peptide is a mutant coagulation factor XII or mutant (poly)peptide or a fragment thereof. In one embodiment, the mutant is a disease-associated mutant of coagulation factor XII or a fragment thereof, which may be used, for example, for preparation of a vaccine to stimulate an immune response. In such a case, a fragment of coagulation factor XII would comprise at least 5, 6, 7, 8 or 9 consecutive amino acid residues of coagulation factor XII to provide an effective immunogen. Preferably, for this purpose the fragment would be a fragment comprising the mutant position of the disease-associated coagulation factor XII (poly)peptide. The use of modified, chimeric peptide constructs and other methods for creating a sufficient immunogenicity are known in the art (see e.g. Rittershaus et al. 2000, Arterioscler. Thromb. Vasc. Biol. 20:2106-2112). Alternatively, it is conceivable to engineer coagulation factor XII in such a way that the resulting mutant can for example displace a disease-associated mutant coagulation factor XII (poly)peptide from one of its interaction partners. Administering such a recombinant, i.e. mutant coagulation factor XII construct to a host may therefore be useful in treating, eventually also in preventing drug-associated angioedema. With respect to a modulator used for the preparation of a pharmaceutical composition and/or a nucleic acid molecule expressing a modulator it is envisaged here that the targeted coagulation factor XII (poly)peptide, or gene or mRNA species, is or contains a disease-associated mutant or mutation.

In a more preferred embodiment of the present invention's use, it is envisaged that the mutant is or is based on: (a) a mutant located in the fibronectin type II domain, within the region of amino acid position 1 to 76, and/or a mutation located in the nucleic acid sequence encoding the fibronectin type II domain, within mRNA position 107 to 334; (b) a mutant located in the EGF-like domain 1, within the region of amino acid position 77 to 113, and/or a mutation located in the nucleic acid sequence encoding the EGF-like domain 1, within mRNA position 335 to 445; (c) a mutant located in the fibronectin type I domain, within the region of amino acid position 114 to 157, and/or a mutation located in the nucleic acid sequence encoding the fibronectin type I domain, within mRNA position 446 to 577; (d) a mutant located in the EGF-like domain 2, within the region of amino acid position 158 to 192, and/or a mutation located in the nucleic acid sequence encoding the EGF-like domain 2, within mRNA position 578 to 682; (e) a mutant located in the kringle domain, within the region of amino acid position 193 to 276, and/or a mutation located in the nucleic acid sequence encoding the kringle domain, within mRNA position 683 to 934; (f) a mutant located in the proline-rich region, within the region of amino acid position 277 to 331, and/or a mutation located in the nucleic acid sequence encoding the proline-rich region, within mRNA position 935 to 1099; (g) a mutant located in the region of proteolytic cleavage sites, within the region of amino acid position 332 to 353, and/or a mutation located in the nucleic acid sequence encoding the region of proteolytic cleavage sites, within mRNA position 1100 to 1165; (h) a mutant located in the serine protease domain, within the region of amino acid position 354 to 596, and/or a mutation located in the nucleic acid sequence encoding the serine protease domain, within mRNA position 1166 to 1894; (i) a mutant located in the signal peptide, within the region of amino acid position −19 to −1, and/or a mutation located in the nucleic acid sequence encoding the signal peptide, within mRNA position 50 to 106; (j) a mutation located in the untranslated regions (UTRs) of coagulation factor XII mRNA, within mRNA position 1 to 49 and/or 1895 to 2048; (k) a mutation located in an intron of the coagulation factor XII gene; and/or (l) a mutation located in a flanking regulatory genomic sequence of the coagulation factor XII gene, within the region encompassing 4000 bp upstream of the transcription initiation site of the coagulation factor XII gene and/or within the region encompassing 3000 bp downstream of the nucleotide sequence representing the 3′-UTR of the coagulation factor XII mRNA. Numbering of sequences etc. is as outlined earlier (vide supra).

Recently, newly identified mutations of the coagulation factor XII gene, namely two mutations in exon 9 encoding the proline-rich region of factor XII (g.6927C>A; g.6927C>G; numbering according to GenBank acc. No. AF 538691), as well as a mutation in intron 2 of the coagulation factor XII gene (g.4278C>T; numbering according to GenBank acc. No. AF 538691) have been found to be significantly associated with a novel type of familial/hereditary angioedema (hereditary angioedema with normal C1 inhibitor, hereditary angioedema type III).

As mentioned earlier (vide supra), it is envisaged that these mutations are also associated with the diseases of the present invention. These mutations may, thus, be useful in accordance with the teaching of the present invention and in particular, in accordance with uses and methods of the present invention. Accordingly, it is envisaged that said mutant located in the proline-rich region is a mutant affecting the threonine residues 309 or 310 of mature coagulation factor XII, more preferably a mutant affecting the Thr309 residue, even more preferably a mutant substituting the Thr309 residue by a lysine or arginine residue, and/or that said mutation located in the nucleic acid sequence encoding the proline-rich region is a mutation within genomic DNA positions 6926 to 6931, more preferably a mutation at position g.6927 and even more preferably a mutation substituting the wild-type C to either an A or a G. Moreover, accordingly, it is also envisaged that said mutation located in an intron of the coagulation factor XII gene is a mutation located in intron 2 of the coagulation factor XII gene, preferably (but not exclusively) a mutation in position 4278 of the genomic sequence given under GenBank acc. No. AF 538691, even more preferably a substitution of the wild-type C by a T (g.4278C>T).

In a more preferred embodiment of the present invention's use, it is envisaged that the modulator is an inhibitor of coagulation factor XII, its activity, its expression and/or its secretion, comprising: (a) an aptamer or an inhibitory antibody or fragment or derivative thereof, specifically binding to and/or specifically inhibiting the activity of (i) disease-associated coagulation factor XII or (ii) wild-type and disease-associated coagulation factor XII; (b) a small molecule inhibitor of (i) disease-associated coagulation factor XII and/or disease-associated coagulation factor XII activity; or (ii) wild-type and disease-associated coagulation factor XII and/or wild-type and disease-associated coagulation factor XII activity; (c) a serine protease inhibitor of (i) disease-associated coagulation factor XII or of (ii) wild-type and disease-associated coagulation factor XII selected from a first group consisting of wild-type and modified or engineered proteinaceous inhibitors of serine proteases including C1 esterase inhibitor, antithrombin III, α2-antiplasmin, α1-antitrypsin, ovalbumin serpins, and α2-macroglobulin, or selected from a second group consisting of Kunitz-type inhibitors including bovine pancreatic trypsin inhibitor; or (d) a siRNA or shRNA, a ribozyme or an antisense nucleic acid molecule specifically hybridizing to a nucleic acid molecule encoding coagulation factor XII or regulating the expression of coagulation factor XII, either affecting (i) disease-associated coagulation factor XII or (ii) wild-type and disease-associated coagulation factor XII. In general, it may be a preferable type of treatment to target specifically the disease-associated mutant coagulation factor XII, its activity, expression and/or secretion. However, it may also be possible to use an inhibitor that targets wild-type as well as disease-associated mutant coagulation factor XII, their activity, expression or secretion; such an option appears particularly reasonable whenever the treatment is not a long-term or ultralong-term treatment.

The present invention also relates to a method of gene therapy in a mammal, characterized by administering an effective amount of a nucleic acid molecule capable of expressing in the mammal: (a) siRNA or shRNA, a ribozyme or an antisense nucleic acid molecule specifically hybridizing to a nucleic acid molecule encoding coagulation factor XII or regulating its expression; (b) an aptamer or an inhibitory antibody or fragment or derivative thereof, specifically binding coagulation factor XII (poly)peptide; (c) coagulation factor XII or a fragment thereof; or (d) a serine protease inhibitor selected from group (i) consisting of wild-type and modified or engineered proteinaceous inhibitors of serine proteases including C1 esterase inhibitor, antithrombin III, α2-antiplasmin, α1-antitrypsin, ovalbumin serpins, and α2-macroglobulin, or selected from group (ii) of Kunitz-type inhibitors including bovine pancreatic trypsin inhibitor.

It is envisaged, for example, that a method of gene therapy of the present invention may be useful for prevention of drug-associated angioedema, e.g. in subjects having a predisposition for drug-associated angioedema that—for medical reasons—must be treated with a certain drug associated with the development of angioedema. Such a method of gene therapy may also be useful with respect to studies on the pathophysiology, treatment and/or prevention of drug-associated angioedema in various animal models, preferably in transgenic animals (as those described below).

The gene therapy method relates to the introduction of nucleic acid sequences, DNA, RNA and/or antisense DNA or RNA sequences, into a mammal. This method requires a nucleic acid construct capable of expressing in the mammal (a) siRNA or shRNA, a ribozyme, or an antisense nucleic acid molecule specifically hybridizing to a nucleic acid molecule encoding or regulating the expression of coagulation factor XII; (b) an aptamer or an inhibitory antibody or fragment or derivative thereof, specifically binding coagulation factor XII (poly)peptide; (c) coagulation factor XII or a fragment thereof; or (d) a proteinaceous serine protease inhibitor, for example C1 esterase inhibitor, antithrombin III, α2-antiplasmin, α2-macroglobulin, α1-antitrypsin, an ovalbumin serpin, or a Kunitz-type inhibitor, modified or engineered in such a way to specifically inhibit coagulation factor XII, preferably disease-associated mutant coagulation factor XII, and any other genetic elements necessary for the expression of the desired (poly)peptide or nucleic acid molecule by the target tissue. Such gene therapy and delivery techniques are known in the art; see, for example, WO90/11092, which is herein incorporated by reference, or: M. I. Phillips (Ed.): Gene Therapy Methods. Methods in Enzymology, Vol. 346, Academic Press, San Diego 2002. Thus, for example, cells from a patient may be engineered ex vivo with a nucleic acid construct comprising a promoter operably linked to the nucleic acid molecule corresponding to the molecule to be introduced, with the engineered cells then being provided to a patient to be treated. Such methods are well-known in the art. For example, see Belldegrun, A., et al., J. Natl. Cancer Inst. 85: 207-216 (1993); Ferrantini, M. et al., Cancer Research 53: 1107-1112 (1993); Ferrantini, M. et al., J. Immunology 153: 4604-4615 (1994); Kaido, T., et al., Int. J. Cancer 60: 221-229 (1995); Ogura, H., et al., Cancer Research 50: 5102-5106 (1990); Santodonato, L., et al., Human Gene Therapy 7:1-10 (1996); Santodonato, L., et al., Gene Therapy 4:1246-1255 (1997); and Zhang, J.-F. et al., Cancer Gene Therapy 3: 31-38 (1996)), which are herein incorporated by reference. The cells which are engineered may be, for example, blood or liver cells. The nucleic acid construct used in gene therapy can be delivered by any method that delivers injectable materials to the cells of an animal, such as, injection into the interstitial space of tissues (heart, muscle, skin, lung, liver, and the like). The nucleic acid molecule used in gene therapy may be delivered in a pharmaceutically acceptable liquid or aqueous carrier.

The nucleic acid molecules may be delivered as a naked nucleic acid molecule. The term “naked” nucleic acid molecule, DNA or RNA refers to sequences that are free from any delivery vehicle that acts to assist, promote or facilitate entry into the cell, including viral sequences, viral particles, liposome formulations, lipofectin or precipitating agents and the like. However, the nucleic acid molecules used in gene therapy can also be delivered in liposome formulations and lipofectin formulations and the like that can be prepared by methods well known to those skilled in the art. Such methods are described, for example, in U.S. Pat. Nos. 5,593,972, 5,589,466, and 5,580,859, which are herein incorporated by reference.

The vector constructs used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF1N5, pcDNA3.1, and pRc/CMV2 available from Invitrogen. Other suitable vectors will be readily apparent to the skilled artisan. Any strong promoter known to those skilled in the art can be used for driving the expression from the nucleic acid molecule used in gene therapy. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the b-actin promoter; and human growth hormone promoters. The promoter also may be the native promoter of coagulation factor XII or of any of the polypeptides expressed in gene therapy. Unlike other gene therapy techniques, one major advantage of introducing naked nucleic acid sequences into target cells is the transitory nature of the nucleic acid molecule synthesis in the cells. Studies have shown that non-replicating DNA sequences can be introduced into cells to provide production of the desired polypeptide for periods of up to six months.

The nucleic acid molecules used in gene therapy can be delivered to the interstitial space of tissues within an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked nucleic acid sequence injection, an effective dosage amount of DNA or RNA will be in the range of from about 0.0005 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid molecules can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose.

The naked nucleic acid molecules are delivered by any method known in the art, including, but not limited to, direct needle injection at the delivery site, intravenous injection, topical administration, catheter infusion, and so-called “gene guns”. These delivery methods are known in the art. The constructs may also be delivered with delivery vehicles such as viral sequences, viral particles, liposome formulations, lipofectin, precipitating agents, etc.

Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Feigner et al., Proc. Natl. Acad. Sci. USA (1987) 84:7413-7416, which is herein incorporated by reference); mRNA (Malone et al., Proc. Natl. Acad. Sci. USA (1989) 86:6077-6081, which is herein incorporated by reference); and purified transcription factors (Debs et al., J. Biol. Chem. (1990) 265:10189-10192, which is herein incorporated by reference), in functional form. Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Feigner et al., Proc. Natl Acad. Sci. USA (1987) 84:7413-7416). Other commercially available liposomes include transfectace (DDAB/DOPE) and DOTAP/DOPE (Boehringer). Other cationic liposomes can be prepared from readily available materials using techniques well known in the art. See, e.g. PCT Publication No. WO 90/11092 (which is herein incorporated by reference) for a description of the synthesis of DOTAP (1,2-bis(oleoyloxy)-3-(trimethylammonio)propane) liposomes. Preparation of DOTMA liposomes is explained in the literature, see, e.g., Feigner et al., Proc. Natl. Acad. Sci. USA 84:7413-7417, which is herein incorporated by reference. Similar methods can be used to prepare liposomes from other cationic lipid materials. Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art. For example, commercially available dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator. Alternatively, negatively charged vesicles can be prepared without sonication to produce multilamellar vesicles or by extrusion through nucleopore membranes to produce unilamellar vesicles of discrete size. Other methods are known and available to those of skill in the art.

Generally, the ratio of nucleic acid to liposomes will be from about 10:1 to about 1:10. Preferably, the ratio will be from about 5:1 to about 1:5. More preferably, the ratio will be about 3:1 to about 1:3. Still more preferably, the ratio will be about 1:1.

In certain embodiments, cells are engineered, ex vivo or in vivo, using a retroviral particle containing RNA which comprises a sequence encoding any of the nucleic acid molecules or (poly)peptides used in the method of gene therapy. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. The retroviral plasmid vector is employed to transduce packaging cell lines to form producer cell lines. Examples of packaging cells which may be transfected include, but are not limited to, the PE501, PA317, R-2, R-AM, PA12, T19-14X, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host. The producer cell line generates infectious retroviral vector particles which include the nucleic acid molecule encoding the (poly)peptide or the therapeutically active nucleic acid, such as siRNA, intended to be used for gene therapy. Such retroviral vector particles then may be employed, to transduce eukaryotic cells, either in vitro or in vivo.

In certain other embodiments, cells are engineered, ex vivo or in vivo, with a nucleic acid molecule to be used in gene therapy, contained in an adenovirus vector. Adenovirus can be manipulated such that it expresses a construct of interest, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis. 109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) Proc. Natl. Acad. Sci. USA 76:6606). Suitable adenoviral vectors useful in the present invention are described, for example, in Kozarsky and Wilson, Curr. Opin. Genet. Devel. 3:499-503 (1993); Rosenfeld et al., Cell 68:143-155 (1992); Engelhardt et al., Human Genet. Ther. 4:759-769 (1993); Yang et al., Nature Genet. 7:362-369 (1994); Wilson et al., Nature 365:691-692 (1993); and U.S. Pat. No. 5,652,224, which are herein incorporated by reference. For example, the adenovirus vector Ad2 is useful and can be grown in human 293 cells. These cells contain the E1 region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the present invention. Preferably, the adenoviruses used in the present invention are replication deficient. Replication deficient adenoviruses require the aid of a helper virus and/or packaging cell line to form infectious particles. The resulting virus is capable of infecting cells and can express a gene of interest which is operably linked to a promoter, but cannot replicate in most cells. Replication deficient adenoviruses may be deleted in one or more of all or a portion of the following genes: E1a, E1b, E3, E4, E2a, or L1 through L5.

The present invention also relates to a non-human transgenic animal, comprising as a transgene: (a) a gene encoding human disease-associated coagulation factor XII; (b) (i) a gene encoding human disease-associated coagulation factor XII and (ii) a gene encoding human wild-type coagulation factor XII; (c) a nucleic acid molecule causing an altered expression of human coagulation factor XII and a gene encoding human wild-type coagulation factor XII; and/or (d) a species-specific coagulation factor XII gene which is specifically altered to contain a human disease-associated mutation.

Said transgenic animal of (a) to (d) will be very important, for example, for studying the pathophysiological consequences of certain coagulation factor XII alterations, in particular in concert with the administration of a drug that is associated with the development of angioedema, and for the screening of new medicaments effective in the treatment and/or prevention of drug-associated angioedema. Preferably, said animal is a mammalian animal, including, but not limited to, rat, mouse, cat, hamster, dog, rabbit, pig, or monkey, but can also be, for example, C. elegans or a fish, such as Torpedo fish.

The non-human transgenic animal of (b) will be valuable for example for studying a heterozygous situation, including possible dominant negative effects of a disease-associated mutation. Further it may allow to investigate potential differential effects of a medicament, including any of the modulators discussed above, on wild-type and disease-associated human coagulation factor XII. The non-human transgenic animal of (c) may allow for example to study the consequences and potential treatment of a mutated nucleic acid that leads to an altered expression of human coagulation factor XII. As envisaged here, such a mutation could relate for example to a nucleic acid molecule which in the human genome is physically unrelated to the coagulation factor XII gene. It is also envisaged that, for example in case of a mutation at a highly conserved position or within a functionally conserved motif, the human disease or disease predisposition can be imitated in the animal by altering the animal's species-specific coagulation factor XII gene to contain a human disease-associated mutation.

A method for the production of a transgenic non-human animal, for example transgenic mouse, comprises introduction of the desired polynucleotide, for example a nucleic acid encoding human wild-type or disease-associated mutant coagulation factor XII, or targeting vector into a germ cell, an embryonic cell, stem cell or an egg or a cell derived therefrom. Production of transgenic embryos and screening of those can be performed, e.g., as described by A. L. Joyner Ed., Gene Targeting, A Practical Approach (1993), Oxford University Press. The DNA of the embryonal membranes of embryos can be analyzed using, e.g., Southern blots with an appropriate probe. A general method for making transgenic non-human animals is described in the art, see for example WO 94/24274. For making transgenic non-human organisms (which include homologously targeted non-human animals), embryonal stem cells (ES cells) are preferred. Murine ES cells, such as AB-1 line grown on mitotically inactive SNL76/7 cell feeder layers (McMahon and Bradley, Cell 62: 1073-1085 (1990)), essentially as described in: Teratocarcinomas and Embryonic Stem Cells: A Practical Approach. E. J. Robertson, ed. (Oxford: IRL Press), 1987, pp. 71-112, may be used for homologous gene targeting. Other suitable ES lines include, but are not limited to, the E14 line (Hooper et al., Nature 326: 292-295 (1987)), the D3 line (Doetschman et al., J. Embryol. Exp. Morph. 87: 27-45 (1985)), the CCE line (Robertson et al., Nature 323: 445-448 (1986)), the AK-7 line (Zhuang et al., Cell 77: 875-884 (1994) which is incorporated by reference herein). The success of generating a mouse line from ES cells bearing a specific targeted mutation depends on the pluripotence of the ES cells (i.e., their ability, once injected into a host developing embryo, such as a blastocyst or morula, to participate in embryogenesis and contribute to the germ cells of the resulting animal). The blastocysts containing the injected ES cells are allowed to develop in the uteri of pseudopregnant nonhuman females and are born as chimeric animals. The resultant transgenic animals are chimeric for cells having either the recombinase or reporter loci and are backcrossed and screened for the presence of the correctly targeted transgene (s) by PCR or Southern blot analysis on tail biopsy DNA of offspring so as to identify transgenic animals heterozygous for either the recombinase or reporter locus/loci.

Methods for producing transgenic flies, such as Drosophila melanogaster are also described in the art, see for example U.S. Pat. No. 4,670,388, Brand & Perrimon, Development (1993) 118: 401-415; and Phelps & Brand, Methods (April 1998) 14: 367-379. Transgenic worms such as C. elegans can be generated as described in Mello, et al., (1991) Efficient gene transfer in C. elegans: extrachromosomal maintenance and integration of transforming sequences. Embo J 10, 3959-70, Plasterk, (1995) Reverse genetics: from gene sequence to mutant worm. Methods Cell Biol 48, 59-80.

In a preferred embodiment of the present invention, the non-human transgenic animal additionally expresses siRNA or shRNA, a ribozyme or an antisense nucleic acid molecule specifically hybridizing to the transgene(s) contained in the transgenic animal. Preferably, said transgene(s) is/are of human origin. Such an approach can be useful, for example, for studying options for treatment and/or prevention for example by using RNA interference.

It may also be desirable to inactivate coagulation factor XII protein expression or function at a certain stage of development and/or life of the transgenic animal. This can be achieved by using, for example, tissue specific, developmental and/or cell regulated and/or inducible promoters which drive the expression of, e.g., an antisense or ribozyme directed against a mRNA encoding a coagulation factor XII (poly)peptide. A suitable inducible system is for example tetracycline-regulated gene expression as described, e.g., by Gossen and Bujard (Proc. Natl. Acad. Sci. 89 USA (1992), 5547-5551) and Gossen et al. (Trends Biotech. 12 (1994), 58-62). Similar, the expression of a mutant coagulation factor XII protein may be controlled by such regulatory elements.

In another preferred embodiment, the non-human transgenic animal's native species-specific genes encoding coagulation factor XII are inactivated. The term “inactivation” means reversible or irreversible inactivation. Appropriate methods to obtain such an inactivation are well known in the art. Such an approach may be useful in order to eliminate any effects of the animal's species-specific coagulation factor XII genes when studying for example the pathophysiological effects and/or the possible therapeutic targeting of the human transgene(s).

The present invention also relates to the use of any of the transgenic animals of the present invention, for screening for compounds for use in the diagnosis, prevention and/or treatment of drug-associated angioedema.

In another preferred embodiment of the present invention, said drug is selected from the group consisting of (a) angiotensin-converting enzyme (ACE) inhibitors; (b) angiotensin II receptor type 1 (AT₁) antagonists (sartans); (c) fibrinolytic or thrombolytic drugs; (d) vasopeptidase inhibitors; (e) neutral endopeptidase (NEP) inhibitors; (f) inhibitors of endothelin-converting enzyme 1 (ECE-1); (g) triple inhibitors of ECE-1, NEP and ACE; (h) inhibitors of dipeptidyl peptidase IV (DPP IV); (i) calcium channel blockers; (j) estrogens and estrogen-like drugs; (k) anti-androgens; and (l) corticosteroids.

With respect to all the aforementioned drugs, as outlined earlier, the present invention also relates to medications that contain these drugs in combination with one or more other substances. In addition, with respect to all these drug classes, related drugs, i.e. drugs with the same biochemical effect or pharmacological mechanism of action (like inhibition of a specific enzyme), are to be considered for the present invention, too.

Finally, the present invention also relates to a kit for use in diagnosis of drug-associated angioedema or a susceptibility or predisposition thereto, said kit comprising: (a) at least one nucleic acid molecule capable of hybridizing under stringent conditions to a nucleic acid molecule encoding or regulating the expression of coagulation factor XII; (b) an antibody or an aptamer specific for coagulation factor XII or a fragment thereof and/or a disease-associated mutant of these; (c) a restriction enzyme capable of discriminating between wild-type and disease-associated mutant nucleic acid encoding or regulating the expression of coagulation factor XII; (d) a pair of primers complementary to nucleic acid regulating the expression of coagulation factor XII or encoding wild-type and/or disease-associated coagulation factor XII; (e) a nucleic acid molecule comprising the human coagulation factor XII nucleotide sequence or a fragment thereof, having a mutation at a position corresponding to position 6927 of GenBank accession no. AF 538691, wherein the wild-type C is substituted by an A or by a G; (f) an oligonucleotide containing at least 8 nucleotides of (fa) the mutant nucleotide sequence of (e) comprising position 6927, wherein the oligonucleotide contains a nucleotide corresponding to mutant position 6927, or the corresponding wild-type sequence of said oligonucleotide or (fb) the complementary sequence of (fa); (g) a (poly)peptide or a fragment thereof, encoded by the nucleic acid molecule of (e); (h) an antibody or antibody fragment specific for the (poly)peptide of (g), wherein said antibody is preferably a monoclonal or polyclonal antibody; and/or (i) a hybridoma producing the monoclonal antibody of (h); and optionally instructions for use. The nucleic acid molecule encoding or regulating the expression of coagulation factor XII of (a) may be a wild-type and/or a disease-associated mutant nucleic acid molecule. The disease-associated mutant or mutation may be any of the mutants or mutations mentioned in the specification of the present invention. Such a kit will be useful with respect to the diagnosis of an angioedema (or a predisposition thereto) related to the use of any of those drugs or drug classes potentially associated with the development of angioedema, preferably with respect to those drugs or drug classes that somehow, directly or indirectly, interact with various components of the kinin system, even more preferably with respect to drug classes (a) to (l) mentioned above. The nucleic acid molecule(s) of (a) may be suitable for example for use as probes or primers. Preferably, the kit will also provide means for detection of a reaction, e.g. nucleotide label detection means, labeled secondary antibodies or size detection means. The various compounds of the kit may be packed in one or more containers, optionally dissolved in suitable buffer for storage.

The Examples illustrate the invention.

### EXAMPLE 1

### The Presence of a Missense Mutation of the Thr309 Residue of Coagulation Factor XII Makes the Carrier Highly Susceptible for the Development of Angioedema (or Angioedema-Related Symptoms) when Using Estrogen-Containing Oral Contraceptives

Among 17 women heterozygous with respect to a missense mutation of the Thr309 residue of coagulation factor XII (either Thr309Lys or Thr309Arg) 15 reported an exposure to estrogen-containing oral contraceptives. 13 of these 15 women developed acute angioedema-related symptoms (skin swellings and/or angioedema-related gastrointestinal symptoms) following the intake of such a medication (symptoms were either precipitated for the first time after starting the OC medication, or a severe exacerbation occurred). Following the switching to an oral contraceptive containing only a gestagen, as done by three patients, symptoms promptly subsided and did not recur.

In contrast to the 13/15 exposed mutation carriers, none out of 12 women with a homozygous wild-type genotype with respect to exon 9 of the F12 gene experienced any angioedema/angioedema-like/angioedema-related symptoms when exposed (for comparable or longer time periods) to estrogen-containing oral contraceptives. This difference is highly significant (p=0.000005; Fisher's exact test), demonstrating that the presence of the Thr309Lys or Thr309Arg mutation of factor XII makes the carrier highly susceptible for the development of angioedema-related symptoms when using estrogen-containing oral contraceptives.

### EXAMPLE 2

### Severe Acute Abdominal Pain Attacks Associated with the Use of Estrogen-Containing Oral Contraceptives: Observations in a Young Woman Heterozygous for the Thr309Lys Mutation of Coagulation Factor XII

The following example of a medical history was given by a female patient heterozygous for the coagulation factor XII Thr309Lys mutation, strongly supporting that estrogen-containing medications (like oral contraceptives) induce angioedema and/or angioedema-related symptoms in association with the presence of a missense mutation the Thr309 residue in exon 9 of the coagulation factor XII gene:

At age 17 years this patient started for the first time to take an estrogen-containing oral contraceptive (OC) (ethinylestradiol 0.035 mg+cyproteronacetate 2 mg). After a few days she began to suffer from acute crampy abdominal pain, together with extreme nausea, vomiting, diarrhoea, accompanied by massive sweating and tachycardia. After seven days she stopped the oral contraceptive, and all symptoms subsided within the next two days. She re-started the same OC with the beginning of the next menstrual cycle. However, because of the re-occurrence of identically severe abdominal symptoms she stopped the OC after four days. Again, all symptoms subsided within the next two days. A few months later she started with a different oral contraceptive (etinylestradiol 0.03 mg+chlormadinonacetat 2 mg). Within three days again extremely severe abdominal symptoms (acute crampy pain, nausea, vomiting, diarrhoea; shock symptoms) occurred, making her to stop the OC after having taken five tablets; all symptoms disappeared within two days. Two months later the patient tried a third oral contraceptive preparation (ethinylestradiol 0.03 mg+dienogest 2 mg). Because of the occurrence of identical symptoms she stopped also this OC already after five days; followed by the prompt resolution of all symptoms.

This patient has not experienced any kind of skin swelling; and symptoms resembling ‘gastrointestinal angioedema’ have only occurred on occasion of the intake of oral contraceptives, as described above.

It is envisaged that the presence of the coagulation factor XII angioedema mutation T309K provides the explanation for the occurrence of the severe OC-related side effect in this patient.

### EXAMPLE 3

### Oligonucleotide Primer Design for Coagulation Factor XII Gene Amplification and Sequencing

Pairs of oligonucleotide primers were designed to amplify the complete human coagulation factor XII gene including flanking sequences. Table 1 lists the corresponding sequences of these primers.

TABLE 1
Oligonucleotide primer sequences
(F = forward, R = reverse)

| Primer ID | Primer Sequence. |
|---|---|
| F12-Ex1-F | 5′-aggaagttgctccacttggct |
| | tt-3′ |
| F12-Ex1-R | 5′-tgcagagatttcttcccaaga |
| | cc-3′ |
| F12-Ex2-F | 5′-ctatgtggaaaggtgaggcca |
| | g-3′ |
| F12-Ex2-R | 5′-ctcaaggatcacacagctcac |
| | g-3′ |
| F12-Ex3-4-F | 5′-tgagggtctgtccttttcctg |
| | a-3′ |
| F12-Ex3-4-R | 5′-ggtgtgtggggtctggtgata |
| | c-3′ |
| F12-Ex5-6-F | 5′-gtaggttcaagaagggccttg |
| | g-3′ |
| F12-Ex5-6-R | 5′-gagctctccttcccggcac-3′ |
| F12-Ex7-F | 5′-gagcagatggttgggaacg-3′ |
| F12-Ex7-R | 5′-tgaggagaaagggggctc-3′ |
| F12-Ex8-F | 5′-ggtctggggcaagcagaag-3′ |
| F12-Ex8-R | 5′-tgtagccacacgacgggg-3′ |
| F12-Ex9-F | 5′-GAACGTGACTGCCGAGCAAG-3′ |
| F12-Ex9-R | 5′-aggagcaggggctgaggac-3′ |
| F12-Ex10-F | 5′-gaaggaggagccgagaggg-3′ |
| F12-Ex10-R | 5′-ggtaggggagaggcagcg-3′ |
| F12-Ex11-12-F | 5′-aggaagctggaacacgggat |
| | t-3′ |
| F12-Ex11-12-R | 5′-ataccaaagtcgcgggcttc |
| | t-3′ |
| F12-Ex13-F | 5′-cccattcaaatcctggcttt |
| | tc-3′ |
| F12-Ex13-R | 5′-AATCACCCTGGGTCGGAAA |
| | C-3′ |
| F12-Ex14-F | 5′-GTGCCAGGTGAGCTCTTAG |
| | CC-3′ |
| F12-Ex14-R | 5′-ccttgttctctgagagctgt |
| | gga-3′ |
| F12-Intr2-pt1-F | 5′-tgtatggtgcagtgtgtgca |
| | gt-3′ |
| F12-Intr2-pt1-R | 5′-ggcatgtaggtaatttagtg |
| | tctggaa-3′ |
| F12-Intr2-pt2-F | 5′-ccttttagatgaagggtacc |
| | tgcc-3′ |
| F12-Intr2-pt2-R | 5′-gagaaacttttgggtgtggg |
| | gt-3′ |
| F12-Intr2-pt3-F | 5′-ctgacttggtggggttgagt |
| | ct-3′ |
| F12-Intr2-pt3-R | 5′-tgccactattttgttcaagg |

ca-3′

| | |
|---|---|
| F12-1ntr2-pt4-F | 5′-ccatttgcatcttaaaggtc |
| | catc-3′ |
| F12-Intr2-pt4-R | 5′-tcacactttgtgcttttgct |
| | gg-3′ |
| F12-Intr2-pt5-F | 5′-acacacgctttctccctaag |
| | gt-3′ |
| F12-Intr2-pt5-R | 5′-ggagtagactcctgactcca |
| | caa-3′ |
| F12-Intr2-pt6-F | 5′-agtattattaagtgcctact |
| | ttgtggc-3′ |
| F12-Intr2-pt6-R | 5′-CAGTGAGAActgcagggaca |
| | ac-3′ |
| F12-Intr4-F | 5′-gaggggactgtgatagggca |
| | g-3′ |
| F12-Intr4-R | 5′-ACACAGGTCCCTCCTTTCTG |
| | G-3′ |
| F12-Intr12-F | 5′-AGACCACGCTCTGCCAGG |
| | T-3′ |
| F12-Intr12-R | 5′-gtaaacccactcatgccctt |
| | cc-3′ |
| F12-P(−1)-F | 5′-cgtcttcttctcatgttcca |
| | gc-3′ |
| F12-P(−1)-R | 5′-actggccaaaggtcttggaa |
| | at-3′ |
| F12-P(−2)-F | 5′-cacagcatctttccatcctt |
| | cc-3′ |
| F12-P(−2)-R | 5′-atcttggggccatcttagca |
| | tt-3′ |
| F12-P(−3)-F | 5′-gtgtcctcacaacacagtgg |
| | ct-3′ |
| F12-P(−3)-R | 5′-cacattgatgatcacctttg |
| | tcac-3′ |
| F12-P(−4)-F | 5′-tgtgcctagccataactgac |
| | ca-3′ |
| F12-P(−4)-R | 5′-tggacttccaagcccagg |
| | t-3′ |
| F12-P(−5)-F | 5′-gtcacgtcaatgactttgaa |
| | acc-3′ |
| F12-P(−5)-R | 5′-cgacatttgagaactagtac |
| | tgatgg-3′ |
| F12-3′UTR-pt1-F | 5′-TCAATAAAGTGCTTTGAAAA |
| | TGCTGA-3′ |
| F12-3′UTR-pt1-R | 5′-tagagacggggtttcatcgt |
| | gt-3′ |
| F12-3′UTR-pt2-F | 5′-gaaatacttagcattggccg |
| | gg-3′ |
| F12-3′UTR-pt2-R | 5′-aaccattcaacccccagatt |
| | gt-3′ |
| F12-Ex9-seqint1-R | 5′-cccccacttcctaacctcc |
| | c-3′ |
| F12-P(−1)-S2-R | 5′-tttgagacggagtctcgc |
| | t-3′ |
| F12-Ex9-ARMS-Mt1-F | 5′-cgccgaagcctcagccca |
| | a-3′ |
| F12-Ex9-ARMS-Mt1-R | 5′-gcgggtcatcgaagacaga |
| | ct-3′ |
| F12-Ex9-RFLP-Mt2-F | 5′-cccggtgtcccctaggctt |
| | c-3′ |
| F12-Ex9-RFLP-Mt2-R | 5′-ctgccggcgcagaaactg |
| | t-3′ |
| F12-Intr10-RFLP-Mt3-F | 5′-aagcgcggaactggggac |
| | t-3′ |
| F12-Intr10-RFLP-Mt3-R | 5′-gctgaacgtaaggcgacag |
| | gag-3′ |

### EXAMPLE 4

### Coagulation Factor XII Gene Amplification and Direct Sequencing of PCR Products

50-100 ng of genomic DNA was amplified by PCR in a total reaction volume of 50 μl containing 2.5 mM MgCl₂, 200 μM each dATP, dCTP, dGTP, dTTP, 5 μl of a 10×PCR buffer (of Invitrogen or Applied Biosystems), 50 μmol of each oligonucleotide primer and 1.25 units Taq DNA polymerase. Occasionally, the buffer had to be optimized by adding denaturing reagents such as DMSO and glycerol or other compounds or compositions known to improve amplification efficiency and specificity.

In general, reactions were thermocycled with an initial denaturation step of 95° C./5 mins [10 min when AmpliTaq Gold DNA polymerase (Applied Biosystems) was used] followed by 35 cycles of 94° C./40 secs; T_{annealing}/40 secs; 72° C./45 secs. For amplimers 20 and 29 subperiods of each cycle of 60 sec/60 sec/120 sec were chosen. A final elongation step of 72° C./10 mins completed the amplification. Annealing temperatures for specific primer pairs and amplimer sizes are presented in Table 2.

Direct sequencing of PCR products was done according to standard procedures (using BigDye™ terminator cycling conditions; purification of reacted products using ethanol precipitation; ABI Automatic Sequencer 3730) known to the skilled artisan (Sambrook et al., “Molecular Cloning, A Laboratory Manual”; ISBN: 0879695765, CSH Press, Cold Spring Harbor, 2001).

TABLE 2
Amplimer sizes and annealing temperatures

| Amplimer | Primer Pair | Size (bp) | Tₐₙₙ (° C.) |
|---|---|---|---|
| 1 | F12-Ex1-F and | 478 | 62° C. |
| | F12-Ex1-R | | |
| 2 | F12-Ex2-F and | 469 | 62° C. |
| | F12-Ex2-R | | |
| 3 | F12-Ex3-4-F and | 504 | 62° C. |
| | F12-Ex3-4-R | | |
| 4 | F12-Ex5-6-F and | 546 | 62° C. |
| | F12-Ex5-6-R | | |
| 5 | F12-Ex7-F and | 386 | 60° C. |
| | F12-Ex7-R | | |
| 6 | F12-Ex8-F and | 386 | 59° C. |
| | F12-Ex8-R | | |
| 7 | F12-Ex9-F and | 459 | 59° C. |
| | F12-Ex9-R | | |
| 8 | F12-Ex10-F and | 550 | 59° C. |
| | F12-Ex10-R | | |
| 9 | F12-Ex11-12-F and | 548 | 60° C. |
| | F12-Ex11-12-R | | |
| 10 | F12-Ex13-F and | 445 | 60° C. |
| | F12-Ex13-R | | |
| 11 | F12-Ex14-F and | 507 | 60° C. |
| | F12-Ex14-R | | |
| 12 | F12-Intr2-pt1-F and | 651 | 63° C. |
| | F12-Intr2-pt1-R | | |
| 13 | F12-Intr2-pt2-F and | 557 | 63° C. |
| | F12-Intr2-pt2-R | | |
| 14 | F12-Intr2-pt3-F and | 598 | 60° C. |
| | F12-Intr2-pt3-R | | |
| 15 | F12-Intr2-pt4-F and | 548 | 57° C. |
| | F12-Intr2-pt4-R | | |
| 16 | F12-Intr2-pt5-F and | 540 | 63° C. |
| | F12-Intr2-pt5-R | | |
| 17 | F12-Intr2-pt6-F and | 584 | 57° C. |
| | F12-Intr2-pt6-R | | |
| 18 | F12-Intr4-F and | 489 | 59° C. |
| | F12-Intr4-R | | |
| 19 | F12-Intr12-F and | 518 | 59° C. |
| | F12-Intr12-R | | |
| 20 | F12-P(-1)-F and | 1275 | 64° C. |
| | F12-P(-1)-R | | |
| 21 | F12-P(-2)-F and | 547 | 62° C. |
| | F12-P(-2)-R | | |
| 22 | F12-P(-3)-F and | 642 | 60° C. |
| | F12-P(-3)-R | | |
| 23 | F12-P(-4)-F and | 442 | 60° C. |
| | F12-P(-4)-R | | |
| 24 | F12-P(-5)-F and | 655 | 60° C. |
| | F12-P(-5)-R | | |
| 25 | F12-3′UTR-pt1-F and | 559 | 58° C. |
| | F12-3′UTR-pt1-R | | |
| 26 | F12-3′UTR-pt2-F and | 559 | 59° C. |
| | F12-3′UTR-pt2-R | | |
| 27 | F12-Ex9-ARMS-Mt1-Fand | 257 | 63° C. |
| | F12-Ex9-ARMS-Mt1-R | | |
| 28 | F12-Ex9-RFLP-Mt2-F and | 390 | 64° C. |
| | F12-Ex9-RFLP-Mt2-R | | |
| 29 | F12-Intr10-RFLP-Mt3-F and | 1204 | 60° C. |
| | F12-Intr10-RFLP-Mt3-R | | |

1. An in vitro method of diagnosing a drug-associated angioedema or a predisposition thereto in a subject being suspected of having developed or of having a predisposition to develop a drug-associated angioedema or in a subject being suspected of being a carrier for a drug-associated angioedema or in a subject being intended to be treated with a drug associated with the development of angioedema, the method comprising determining in a biological sample from said subject the presence or absence of a disease-associated mutation in a nucleic acid molecule regulating the expression of or encoding coagulation factor XII; wherein the presence of such a mutation is indicative of a drug-associated angioedema or a predisposition thereto.

2. The method of claim 1, wherein said determination comprises hybridizing under stringent conditions to said nucleic acid molecule at least one pair of nucleic acid probes, the first probe of said pair being complementary to the wild-type sequence of said nucleic acid molecule and the second probe of said pair being complementary to the mutant sequence of said nucleic acid molecule, wherein a perfect match, the presence of stable hybridization, between (i) the first hybridization probe and the target nucleic acid molecule indicates the presence of a wild-type sequence, and (ii) the second hybridization probe and the target nucleic acid molecule, indicates the presence of a mutant sequence, wherein the first hybridization probe and the second hybridization probe allow a differential detection.

3. The method of claim 1, said method comprising hybridizing under stringent conditions to said nucleic acid molecule a hybridization probe specific for a mutant sequence.

4. The method of claim 1, comprising a step of nucleic acid amplification and/or nucleic acid sequencing.

5. The method of claim 1, wherein the method is or comprises an allele discrimination method selected from the group consisting of allele-specific hybridization, allele-specific primer extension including allele-specific PCR, allele-specific oligonucleotide ligation, allele-specific cleavage of a flap probe and/or allele-specific cleavage using a restriction endonuclease.

6. The method of claim 1, comprising a detection method selected from the group consisting of fluorescence detection, time-resolved fluorescence, fluorescence resonance energy transfer (FRET), fluorescence polarization, colorimetric methods, mass spectrometry, (chemi)luminescence, electrophoretical detection and electrical detection methods.

7. The method of claim 1, wherein the probe or the subject's nucleic acid molecule is attached to a solid support.

8. A method of diagnosing a drug-associated angioedema or a predisposition thereto in a subject being suspected of having developed or of having a predisposition to develop a drug-associated angioedema or in a subject being suspected of being a carrier for a drug-associated angioedema or in a subject being intended to be treated with a drug associated with the development of angioedema, the method comprising assessing the presence, amount and/or activity of coagulation factor XII in said subject and including the steps of:
(a) determining from a biological sample of said subject in vitro, the presence, amount and/or activity of:
(i.) a (poly)peptide encoded by the coagulation factor XII gene;
(ii.) a substrate of the (poly)peptide of (i); or
(iii.) a (poly)peptide processed by the substrate mentioned in (ii);
(b) comparing said presence, amount and/or activity with that determined from a reference sample; and
(c) diagnosing, based on the difference between the samples compared in step (b), the pathological condition of a drug-associated angioedema or a predisposition thereto.

9. The method of claim 1, wherein the biological sample consists of or is taken from hair, skin, mucosal surfaces, body fluids, including blood, plasma, serum, urine, saliva, sputum, tears, liquor cerebrospinalis, semen, synovial fluid, amniotic fluid, milk, lymph, pulmonary sputum, bronchial secretion, or stool.

10. The method of claim 8, wherein said presence, amount and/or activity is determined by using an antibody or an aptamer, wherein the antibody or aptamer is specific for (a) a (poly)peptide encoded by the coagulation factor XII gene; (b) a substrate of the (poly)peptide of (a); or (c) a (poly)peptide processed by the substrate mentioned in (b).

11. The method of claim 10, wherein said antibody or aptamer is specific for a (poly)peptide encoded by the coagulation factor XII gene.

12. The method of claim 8, wherein the presence, amount and/or activity of the (poly)peptide(s) encoded by the coagulation factor XII gene is determined in (a) a coagulation assay; or in (b) a functional amidolytic assay; or in (c) a mitogenic assay; or in (d) a binding assay measuring binding of a (poly)peptide encoded by the coagulation factor XII gene to a binding partner.

13. A method of identifying a compound modulating coagulation factor XII activity which is suitable as a medicament or a lead compound for a medicament for the treatment and/or prevention of drug-associated angioedema, the method comprising the steps of:
(a) in vitro contacting a coagulation factor XII (poly)peptide or a functionally related (poly)peptide with the potential modulator; and
(b) testing for modulation of coagulation factor XII activity,
wherein modulation of coagulation factor XII activity is indicative of a compound's suitability as a medicament or a lead compound for a medicament for the treatment and/or prevention of drug-associated angioedema.

14. The method of claim 13, wherein the coagulation factor XII (poly)peptide of step (a) is present in cell culture or cell culture supernatant or in a subject's sample or purified from any of these sources.

15. The method of claim 13, wherein said testing is performed by assessing the physical interaction between a coagulation factor XII (poly)peptide and the modulator and/or the effect of the modulator on the function of said coagulation factor XII (poly)peptide.

16. The method of claim 13, wherein the modulator is an inhibitor of coagulation factor XII activity, selected from the group consisting of:
(a) an aptamer or inhibitory antibody or fragment or derivative thereof, specifically binding to a coagulation factor XII (poly)peptide and/or specifically inhibiting a coagulation factor XII activity;
(b) a small molecule inhibitor of coagulation factor XII and/or coagulation factor XII activity; and
(c) a serine protease inhibitor selected from group (I) consisting of wild-type and modified or engineered proteinaceous inhibitors of serine proteases including C1 esterase inhibitor, antithrombin III, α2-antiplasmin, α1-antitrypsin, ovalbumin serpins, and α2-macroglobulin, or selected from group (II) of Kunitz-type inhibitors including bovine pancreatic trypsin inhibitor.

17. A method of identifying a compound modulating coagulation factor XII expression and/or secretion which is suitable as a medicament or lead compound for a medicament for the treatment and/or prevention of drug-associated angioedema, the method comprising the steps of:
(a) in vitro contacting a cell that expresses or is capable of expressing coagulation factor XII with a potential modulator of expression and/or secretion; and
(b) testing for altered expression and/or secretion,
wherein the modulator is (i) a small molecule compound, an aptamer or an antibody or fragment or derivative thereof, specifically modulating expression and/or secretion of coagulation factor XII; or (ii) a siRNA or shRNA, a ribozyme, or an antisense nucleic acid molecule specifically hybridizing to a nucleic acid molecule encoding coagulation factor XII or regulating the expression of coagulation factor XII.

18. The method of claim 13, wherein coagulation factor XII is a disease-associated mutant of coagulation factor XII.

19. The method of claim 13, wherein said modulator is selective for a disease-associated mutant of coagulation factor XII, the method comprising (a) comparing the effect of the modulator on wild-type and disease-associated coagulation factor XII activity or their expression and/or secretion; and (b) selecting a compound which (i) modulates disease-associated coagulation factor XII activity or its expression and/or secretion and which (ii) does not affect wild-type coagulation factor XII activity or its expression and/or secretion.

20. The method of claim 1, wherein the disease-associated mutant or mutation is:
(a) a mutant located in the fibronectin type II domain, within the region of amino acid position 1 to 76, and/or a mutation located in the nucleic acid sequence encoding the fibronectin type II domain, within mRNA position 107 to 334;
(b) a mutant located in the EGF-like domain 1, within the region of amino acid position 77 to 113, and/or a mutation located in the nucleic acid sequence encoding the EGF-like domain 1, within mRNA position 335 to 445;
(c) a mutant located in the fibronectin type I domain, within the region of amino acid position 114 to 157, and/or a mutation located in the nucleic acid sequence encoding the fibronectin type I domain, within mRNA position 446 to 577;
(d) a mutant located in the EGF-like domain 2, within the region of amino acid position 158 to 192, and/or a mutation located in the nucleic acid sequence encoding the EGF-like domain 2, within mRNA position 578 to 682;
(e) a mutant located in the kringle domain, within the region of amino acid position 193 to 276, and/or a mutation located in the nucleic acid sequence encoding the kringle domain, within mRNA position 683 to 934;
(f) a mutant located in the proline-rich region, within the region of amino acid position 277 to 331, and/or a mutation located in the nucleic acid sequence encoding the proline-rich region, within mRNA position 935 to 1099;
(g) a mutant located in the region of proteolytic cleavage sites, within the region of amino acid position 332 to 353, and/or a mutation located in the nucleic acid sequence encoding the region of proteolytic cleavage sites, within mRNA position 1100 to 1165;
(h) a mutant located in the serine protease domain, within the region of amino acid position 354 to 596, and/or a mutation located in the nucleic acid sequence encoding the serine protease domain, within mRNA position 1166 to 1894;
(i) a mutant located in the signal peptide, within the region of amino acid position −19 to −1, and/or a mutation located in the nucleic acid sequence encoding the signal peptide, within mRNA position 50 to 106;
(j) a mutation located in the untranslated regions (UTRs) of coagulation factor XII mRNA, within mRNA position 1 to 49 and/or 1895 to 2048;
(k) a mutation located in an intron of the coagulation factor XII gene; and/or
(l) a mutation located in a flanking regulatory genomic sequence of the coagulation factor XII gene, within the region encompassing 4000 bp upstream of the transcription initiation site of the coagulation factor XII gene and/or within the region encompassing 3000 bp downstream of the nucleotide sequence representing the 3′-UTR of the coagulation factor XII mRNA.

21. The method of claim 20, wherein said mutant in (f) is a mutant affecting amino acid residue 309 or 310.

22. The method of claim 21, wherein said amino acid residue at position 309 is substituted by a basic or positively charged amino acid residue.

23. The method of claim 22, wherein said basic or positively charged amino acid residue is a lysine or arginine.

24. (canceled)

25. The method of claim 1, comprising in vitro testing of a sample of a blood donor for determining whether the blood of said donor or components thereof may be used for transfusion to a patient in need thereof, wherein a positive testing indicates a predisposition for drug-associated angioedema, excluding the transfusion of blood or components thereof from said donor.

26. The method of claim 1, wherein said drug is selected from the group consisting of (a) angiotensin-converting enzyme (ACE) inhibitors; (b) angiotensin II receptor type 1 (AT₁) antagonists (sartans); (c) fibrinolytic or thrombolytic drugs; (d) vasopeptidase inhibitors; (e) neutral endopeptidase (NEP) inhibitors; (f) inhibitors of endothelin-converting enzyme 1 (ECE-1); (g) triple inhibitors of ECE-1, NEP and ACE; (h) inhibitors of dipeptidyl peptidase IV (DPP IV); (i) calcium channel blockers; (j) estrogens and estrogen-like drugs; (k) anti-androgens; and (l) corticosteroids.

27. Use of (a) a (poly)peptide encoded by the coagulation factor XII gene or a fragment thereof, (b) a nucleic acid molecule capable of expressing coagulation factor XII or a fragment thereof; and/or (c) a nucleic acid molecule capable of expressing a modulator of coagulation factor XII activity or its expression and/or secretion, for the preparation of a pharmaceutical composition for the treatment and/or prevention of drug-associated angioedema.

28. The use of claim 27, wherein said coagulation factor XII or said (poly)peptide is a mutant coagulation factor XII or mutant (poly)peptide or a fragment thereof.

29. The use of claim 28, wherein said mutant is or is based on:
(a) a mutant located in the fibronectin type II domain, within the region of amino acid position 1 to 76, and/or a mutation located in the nucleic acid sequence encoding the fibronectin type II domain, within mRNA position 107 to 334;
(b) a mutant located in the EGF-like domain 1, within the region of amino acid position 77 to 113, and/or a mutation located in the nucleic acid sequence encoding the EGF-like domain 1, within mRNA position 335 to 445;
(c) a mutant located in the fibronectin type I domain, within the region of amino acid position 114 to 157, and/or a mutation located in the nucleic acid sequence encoding the fibronectin type I domain, within mRNA position 446 to 577;
(d) a mutant located in the EGF-like domain 2, within the region of amino acid position 158 to 192, and/or a mutation located in the nucleic acid sequence encoding the EGF-like domain 2, within mRNA position 578 to 682
(e) a mutant located in the kringle domain, within the region of amino acid position 193 to 276, and/or a mutation located in the nucleic acid sequence encoding the kringle domain, within mRNA position 683 to 934;
(f) a mutant located in the proline-rich region, within the region of amino acid position 277 to 331, and/or a mutation located in the nucleic acid sequence encoding the proline-rich region, within mRNA position 935 to 1099;
(g) a mutant located in the region of proteolytic cleavage sites, within the region of amino acid position 332 to 353, and/or a mutation located in the nucleic acid sequence encoding the region of proteolytic cleavage sites, within mRNA position 1100 to 1165;
(h) a mutant located in the serine protease domain, within the region of amino acid position 354 to 596, and/or a mutation located in the nucleic acid sequence encoding the serine protease domain, within mRNA position 1166 to 1894;
(i) a mutant located in the signal peptide, within the region of amino acid position −19 to −1, and/or a mutation located in the nucleic acid sequence encoding the signal peptide, within mRNA position 50 to 106;
(j) a mutation located in the untranslated regions (UTRs) of coagulation factor XII mRNA, within mRNA position 1 to 49 and/or 1895 to 2048;
(k) a mutation located in an intron of the coagulation factor XII gene; and/or
(l) a mutation located in a flanking regulatory genomic sequence of the coagulation factor XII gene, within the region encompassing 4000 bp upstream of the transcription initiation site of the coagulation factor XII gene and/or within the region encompassing 3000 bp downstream of the nucleotide sequence representing the 3′-UTR of the coagulation factor XII mRNA.

30. The use of claim 29, wherein said mutant in (f) is a mutant affecting amino acid residue 309 or 310.

31. The use of claim 30, wherein said amino acid residue at position 309 is substituted by a basic or positively charged amino acid residue.

32. The use of claim 31, wherein said basic or positively charged amino acid residue is a lysine or arginine.

33. The use of claim 27, wherein said modulator is an inhibitor of coagulation factor XII, its activity, its expression and/or its secretion, comprising:
(a) an aptamer or an inhibitory antibody or fragment or derivative thereof, specifically binding to and/or specifically inhibiting the activity of (i) disease-associated coagulation factor XII or (ii) wild-type and disease-associated coagulation factor XII;
(b) a small molecule inhibitor of (i) disease-associated coagulation factor XII and/or disease-associated coagulation factor XII activity; or (ii) wild-type and disease-associated coagulation factor XII and/or wild-type and disease-associated coagulation factor XII activity;
(c) a serine protease inhibitor of (i) disease-associated coagulation factor XII or of (ii) wild-type and disease-associated coagulation factor XII selected from a first group consisting of wild-type and modified or engineered proteinaceous inhibitors of serine proteases including C1 esterase inhibitor, antithrombin III, a 2-antiplasmin, a 1-antitrypsin, ovalbumin serpins, and a 2-macroglobulin, or selected from a second group consisting of Kunitz-type inhibitors including bovine pancreatic trypsin inhibitor; or
(d) a siRNA or shRNA, a ribozyme or an antisense nucleic acid molecule specifically hybridizing to a nucleic acid molecule encoding coagulation factor XII or regulating the expression of coagulation factor XII, either affecting (i) disease-associated coagulation factor XII or (ii) wild-type and disease-associated coagulation factor XII.

34. (canceled)

35. A non-human transgenic animal, comprising as a transgene:
(a) a gene encoding human disease-associated coagulation factor XII;
(b) (i) a gene encoding human disease-associated coagulation factor XII and (ii) a gene encoding human wild-type coagulation factor XII;
(c) a nucleic acid molecule causing an altered expression of human coagulation factor XII and a gene encoding human wild-type coagulation factor XII; and/or
(d) a species-specific coagulation factor XII gene which is specifically altered to contain a human disease-associated mutation.

36.-38. (canceled)

39. The use of claim 27, wherein said drug is selected from the group consisting of (a) angiotensin-converting enzyme (ACE) inhibitors; (b) angiotensin II receptor type 1 (AT_{I}) antagonists (sartans); (c) fibrinolytic or thrombolytic drugs; (d) vasopeptidase inhibitors; (e) neutral endopeptidase (NEP) inhibitors; (f) inhibitors of endothelin-converting enzyme 1 (ECE-1); (g) triple inhibitors of ECE-1, NEP and ACE; (h) inhibitors of dipeptidyl peptidase IV (DPP IV); (i) calcium channel blockers; (j) estrogens and estrogen-like drugs; (k) anti-androgens; and (1) corticosteroids.

40. A kit for use in diagnosis of drug-associated angioedema or a susceptibility or predisposition thereto, said kit comprising:
(a) at least one nucleic acid molecule capable of hybridizing under stringent conditions to a nucleic acid molecule encoding or regulating the expression of coagulation factor XII;
(b) an antibody or an aptamer specific for coagulation factor XII or a fragment thereof and/or a disease-associated mutant of these;
(c) a restriction enzyme capable of discriminating between wild-type and disease-associated mutant nucleic acid encoding or regulating the expression of coagulation factor XII;
(d) a pair of primers complementary to nucleic acid regulating the expression of coagulation factor XII or encoding wild-type and/or disease-associated coagulation factor XII;
(e) a nucleic acid molecule comprising the human coagulation factor XII nucleotide sequence or a fragment thereof, having a mutation at a position corresponding to position 6927 of GenBank accession no. AF 538691, wherein the wild-type C is substituted by an A or by a G;
(f) an oligonucleotide containing at least 8 nucleotides of (fa) the mutant nucleotide sequence of (e) comprising position 6927, wherein the oligonucleotide contains a nucleotide corresponding to mutant position 6927, or the corresponding wild-type sequence of said oligonucleotide or (fb) the complementary sequence of (fa);
(g) a (poly)peptide or a fragment thereof, encoded by the nucleic acid molecule of (e);
(h) an antibody or antibody fragment specific for the (poly)peptide of (g), wherein said antibody is preferably a monoclonal or polyclonal antibody; and/or
(i) a hybridoma producing the monoclonal antibody of (h);
and optionally instructions for use.

41.-42. (canceled)


SEQUENCE LISTING (60 sequences)

SEQ ID NO: 1 - DNA, 23 bp, Artificial Sequence
  Description of Artificial Sequence Oligonucleotide primer F12-Ex1-F
aggaagttgctccacttggcttt

SEQ ID NO: 2 - DNA, 23 bp, Artificial Sequence
  Description of Artificial Sequence Oligonucleotide primer F12-Ex1-R
tgcagagatttcttcccaagacc

SEQ ID NO: 3 - DNA, 22 bp, Artificial Sequence
  Description of Artificial Sequence Oligonucleotide primer F12-Ex2-F
ctatgtggaaaggtgaggccag

SEQ ID NO: 4 - DNA, 22 bp, Artificial Sequence
  Description of Artificial Sequence Oligonucleotide primer F12-Ex2-R
ctcaaggatcacacagctcacg

SEQ ID NO: 5 - DNA, 22 bp, Artificial Sequence
  Description of Artificial Sequence Oligonucleotide primer F12-Ex3-4-F
tgagggtctgtccttttcctga

SEQ ID NO: 6 - DNA, 22 bp, Artificial Sequence
  Description of Artificial Sequence Oligonucleotide primer F12-Ex3-4-R
ggtgtgtggggtctggtgatac

SEQ ID NO: 7 - DNA, 22 bp, Artificial Sequence
  Description of Artificial Sequence Oligonucleotide primer F12-Ex5-6-F
gtaggttcaagaagggccttgg

SEQ ID NO: 8 - DNA, 19 bp, Artificial Sequence
  Description of Artificial Sequence Oligonucleotide primer F12-Ex5-6-R
gagctctccttcccggcac

SEQ ID NO: 9 - DNA, 19 bp, Artificial Sequence
  Description of Artificial Sequence Oligonucleotide primer F12-Ex7-F
gagcagatggttgggaacg

SEQ ID NO: 10 - DNA, 18 bp, Artificial Sequence
  Description of Artificial Sequence Oligonucleotide primer F12-Ex7-R
tgaggagaaagggggctc

SEQ ID NO: 11 - DNA, 19 bp, Artificial Sequence
  Description of Artificial Sequence Oligonucleotide primer F12-Ex8-F
ggtctggggcaagcagaag

SEQ ID NO: 12 - DNA, 18 bp, Artificial Sequence
  Description of Artificial Sequence Oligonucleotide primer F12-Ex8-R
tgtagccacacgacgggg

SEQ ID NO: 13 - DNA, 20 bp, Artificial Sequence
  Description of Artificial Sequence Oligonucleotide primer F12-Ex9-F
gaacgtgactgccgagcaag

SEQ ID NO: 14 - DNA, 19 bp, Artificial Sequence
  Description of Artificial Sequence Oligonucleotide primer F12-Ex9-R
aggagcaggggctgaggac

SEQ ID NO: 15 - DNA, 19 bp, Artificial Sequence
  Description of Artificial Sequence Oligonucleotide primer F12-Ex10-F
gaaggaggagccgagaggg

SEQ ID NO: 16 - DNA, 18 bp, Artificial Sequence
  Description of Artificial Sequence Oligonucleotide primer F12-Ex10-R
ggtaggggagaggcagcg

SEQ ID NO: 17 - DNA, 21 bp, Artificial Sequence
  Description of Artificial Sequence Oligonucleotide primer F12-Ex11-12-F
aggaagctggaacacgggatt

SEQ ID NO: 18 - DNA, 21 bp, Artificial Sequence
  Description of Artificial Sequence Oligonucleotide primer F12-Ex11-12-R
ataccaaagtcgcgggcttct

SEQ ID NO: 19 - DNA, 22 bp, Artificial Sequence
  Description of Artificial Sequence Oligonucleotide primer F12-Ex13-F
cccattcaaatcctggcttttc

SEQ ID NO: 20 - DNA, 20 bp, Artificial Sequence
  Description of Artificial Sequence Oligonucleotide primer F12-Ex13-R
aatcaccctgggtcggaaac

SEQ ID NO: 21 - DNA, 21 bp, Artificial Sequence
  Description of Artificial Sequence Oligonucleotide primer F12-Ex14-F
gtgccaggtgagctcttagcc

SEQ ID NO: 22 - DNA, 23 bp, Artificial Sequence
  Description of Artificial Sequence Oligonucleotide primer F12-Ex14-R
ccttgttctctgagagctgtgga

SEQ ID NO: 23 - DNA, 22 bp, Artificial Sequence
  Description of Artificial Sequence Oligonucleotide primer F12-Intr2-pt1-F
tgtatggtgcagtgtgtgcagt

SEQ ID NO: 24 - DNA, 27 bp, Artificial Sequence
  Description of Artificial Sequence Oligonucleotide primer F12-Intr2-pt1-R
ggcatgtaggtaatttagtgtctggaa

SEQ ID NO: 25 - DNA, 24 bp, Artificial Sequence
  Description of Artificial Sequence Oligonucleotide primer F12-Intr2-pt2-F
ccttttagatgaagggtacctgcc

SEQ ID NO: 26 - DNA, 22 bp, Artificial Sequence
  Description of Artificial Sequence Oligonucleotide primer F12-Intr2-pt2-R
gagaaacttttgggtgtggggt

SEQ ID NO: 27 - DNA, 22 bp, Artificial Sequence
  Description of Artificial Sequence Oligonucleotide primer F12-Intr2-pt3-F
ctgacttggtggggttgagtct

SEQ ID NO: 28 - DNA, 22 bp, Artificial Sequence
  Description of Artificial Sequence Oligonucleotide primer F12-Intr2-pt3-R
tgccactattttgttcaaggca

SEQ ID NO: 29 - DNA, 24 bp, Artificial Sequence
  Description of Artificial Sequence Oligonucleotide primer F12-Intr2-pt4-F
ccatttgcatcttaaaggtccatc

SEQ ID NO: 30 - DNA, 22 bp, Artificial Sequence
  Description of Artificial Sequence Oligonucleotide primer F12-Intr2-pt4-R
tcacactttgtgcttttgctgg

SEQ ID NO: 31 - DNA, 22 bp, Artificial Aequence
  Description of Artificial Sequence Oligonucleotide primer F12-Intr2-pt5-F
acacacgctttctccctaaggt

SEQ ID NO: 32 - DNA, 23 bp, Artificial Sequence
  Description of Artificial Sequence Oligonucleotide primer F12-Intr2-pt5-R
ggagtagactcctgactccacaa

SEQ ID NO: 33 - DNA, 27 bp, Artificial Sequence
  Description of Artificial Sequence Oligonucleotide primer F12-Intr2-pt6-F
agtattattaagtgcctactttgtggc

SEQ ID NO: 34 - DNA, 22 bp, Artificial Sequence
  Description of Artificial Sequence Oligonucleotide primer F12-Intr2-pt6-R
cagtgagaactgcagggacaac

SEQ ID NO: 35 - DNA, 21 bp, Artificial Sequence
  Description of Artificial Sequence Oligonucleotide primer F12-Intr4-F
gaggggactgtgatagggcag

SEQ ID NO: 36 - DNA, 21 bp, Artificial Sequence
  Description of Artificial Sequence Oligonucleotide primer F12-Intr4-R
acacaggtccctcctttctgg

SEQ ID NO: 37 - DNA, 19 bp, Artificial Sequence
  Description of Artificial Sequence Oligonucleotide primer F12-Intr12-F
agaccacgctctgccaggt

SEQ ID NO: 38 - DNA, 22 bp, Artificial Sequence
  Description of Artificial Sequence Oligonucleotide primer F12-Intr12-R
gtaaacccactcatgcccttcc

SEQ ID NO: 39 - DNA, 22 bp, Artificial Sequence
  Description of Artificial Sequence Oligonucleotide primer F12-P(-1)-F
cgtcttcttctcatgttccagc

SEQ ID NO: 40 - DNA, 22 bp, Artificial Sequence
  Description of Artificial Sequence Oligonucleotide primer F12-P(-1)-R
actggccaaaggtcttggaaat

SEQ ID NO: 41 - DNA, 22 bp, Artificial Sequence
  Description of Artificial Sequence Oligonucleotide primer F12-P(-2)-F
cacagcatctttccatccttcc

SEQ ID NO: 42 - DNA, 22 bp, Artificial Sequence
  Description of Artificial Sequence Oligonucleotide primer F12-P(-2)-R
atcttggggccatcttagcatt

SEQ ID NO: 43 - DNA, 22 bp, Artificial Sequence
  Description of Artificial Sequence Oligonucleotide primer F12-P(-3)-F
gtgtcctcacaacacagtggct

SEQ ID NO: 44 - DNA, 24 bp, Artificial Sequence
  Description of Artificial Sequence Oligonucleotide primer F12-P(-3)-R
cacattgatgatcacctttgtcac

SEQ ID NO: 45 - DNA, 19 bp, Artificial Sequence
  Description of Artificial Sequence Oligonucleotide primer F12-P(-4)-F
tggacttccaagcccaggt

SEQ ID NO: 46 - DNA, 19 bp, Artificial Sequence
  Description of Artificial Sequence Oligonucleotide primer F12-P(-4)-R
tggacttccaagcccaggt

SEQ ID NO: 47 - DNA, 23 bp, Artificial Sequence
  Description of Artificial Sequence Oligonucleotide primer F12-P(-5)-F
gtcacgtcaatgactttgaaacc

SEQ ID NO: 48 - DNA, 26 bp, Artificial Sequence
  Description of Artificial Sequence Oligonucleotide primer F12-P(-5)-R
cgacatttgagaactagtactgatgg

SEQ ID NO: 49 - DNA, 26 bp, Artificial Sequence
  Description of Artificial Sequence Oligonucleotide primer F12-3′UTR-pt1-F
tcaataaagtgctttgaaaatgctga

SEQ ID NO: 50 - DNA, 22 bp, Artificial Sequence
  Description of Artificial Sequence Oligonucleotide primer F12-3′UTR-pt1-R
tagagacggggtttcatcgtgt

SEQ ID NO: 51 - DNA, 22 bp, Artificial Sequence
  Description of Artificial Sequence Oligonucleotide primer F12-3′UTR-pt2-F
gaaatacttagcattggccggg

SEQ ID NO: 52 - DNA, 22 bp, Artificial Sequence
  Description of Artificial Sequence Oligonucleotide primer F12-3′UTR-pt2-R
aaccattcaacccccagattgt

SEQ ID NO: 53 - DNA, 20 bp, Artificial Sequence
  Description of Artificial Sequence Oligonucleotide primer F12-Ex9-seqint1-R
cccccacttcctaacctccc

SEQ ID NO: 54 - DNA, 19 bp, Artificial Sequence
  Description of Artificial Sequence Oligonucleotide primer F12-P(-1)-S2-R
tttgagacggagtctcgct

SEQ ID NO: 55 - DNA, 19 bp, Artificial Sequence
  Description of Artificial Sequence Oligonucleotide primer F12-Ex9-ARMS-Mt1-F
cgccgaagcctcagcccaa

SEQ ID NO: 56 - DNA, 21 bp, Artificial Aequence
  Description of Artificial Sequence Oligonucleotide primer F12-Ex9-ARMS-Mt1-R
gcgggtcatcgaagacagact

SEQ ID NO: 57 - DNA, 20 bp, Artificial Sequence
  Description of Artificial Sequence Oligonucleotide primer F12-Ex9-RFLP-Mt2-F
cccggtgtcccctaggcttc

SEQ ID NO: 58 - DNA, 19 bp, Artificial Sequence
  Description of Artificial Sequence Oligonucleotide primer F12-Ex9-RFLP-Mt2-R
ctgccggcgcagaaactgt

SEQ ID NO: 59 - DNA, 19 bp, Artificial Sequence
  Description of Artificial Sequence Oligonucleotide primer F12-Intr10-RFLP-Mt3-F
aagcgcggaactggggact

SEQ ID NO: 60 - DNA, 22 bp, Artificial Sequence
  Description of Artificial Sequence Oligonucleotide primer F12-Intr10-RFLP-Mt3-R
gctgaacgtaaggcgacaggag